(19) United States
(12) Patent Application Publication
(10) Pub. No.: US 2013/0276161 A1
(43) Pub. Date: Oct. 17, 2013

(54) TRANSGENIC PLANTS WITH ENHANCED AGRONOMIC TRAITS

(71) Applicants: Mark Abad, Webster Groves, MO (US); Erin Bell, St. Louis, MO (US); R. Eric Cerny, Chesterfield, MO (US); Jaishree M. Chittoor-Vijayanath, Wildwood, MO (US); Molian Deng, Grover, MO (US); Michael D. Edgerton, St. Louis, MO (US); Karen K. Gabbert, St. Louis, MO (US); Barry S. Goldman, St. Louis, MO (US); Brian Hauge, Wildwood, MO (US); Steve He, St. Louis, MO (US); Jacqueline E. Heard, Webster Groves, MO (US); Ping Li, St. Peters, MO (US); Linda Lutfiyya, St. Louis, MO (US); Bradley McDill, Smithton, IL (US); Robert J. Meister, St. Peters, MO (US); Peter Morrell, Chesterfield, MO (US); Anil Neelam, Wildwood, MO (US); Marie Petracek, Glendale, MO (US); John Pitkin, Wildwood, MO (US); M. S. Rajani, St. Louis, MO (US); Maria F. Rodriguez, Kirkwood, MO (US); Thomas Ruff, Wildwood, MO (US); T. V. Venkatesh, St. Louis, MO (US); Xiaoyun Wu, Chesterfield, MO (US); Jianmin Zhao, St. Louis, MO (US); Albert Iandolino, Davis, CA (US); Monica Ravanello, Fairfield, CA (US); Kraig Brustad, Stonington, CT (US); Paul Chomet, Mystic, CT (US); Julie Davis, North Stonington, CT (US); Virginia Ursin, Pawcatuck, CT (US); T. Ambika, Bangalore (IN); Shobha Char, Bangalore (IN); Savitha Madappa, Bangalore (IN); Dhanalakshmi Ramachandra, Bangalore (IN); S. Sangeetha, Bangalore (IN); K. R. Vidya, Bangalore (IN); K. Vijayalakshmi, Bangalore (IN); Todd Dezwaan, Apex, NC (US)

(72) Inventors: Mark Abad, Webster Groves, MO (US); Erin Bell, St. Louis, MO (US); R. Eric Cerny, Chesterfield, MO (US); Jaishree M. Chittoor-Vijayanath, Wildwood, MO (US); Molian Deng, Grover, MO (US); Michael D. Edgerton, St. Louis, MO (US); Karen K. Gabbert, St. Louis, MO (US); Barry S. Goldman, St. Louis, MO (US); Brian Hauge, Wildwood, MO (US); Steve He, St. Louis, MO (US); Jacqueline E. Heard, Webster Groves, MO (US); Ping Li, St. Peters, MO (US); Linda Lutfiyya, St. Louis, MO (US); Bradley McDill, Smithton, IL (US); Robert J. Meister, St. Peters, MO (US); Peter Morrell, Chesterfield, MO (US); Anil Neelam, Wildwood, MO (US); Marie Petracek, Glendale, MO (US); John Pitkin, Wildwood, MO (US); M. S. Rajani, St. Louis, MO (US); Maria F. Rodriguez, Kirkwood, MO (US); Thomas Ruff, Wildwood, MO (US); T. V. Venkatesh, St. Louis, MO (US); Xiaoyun Wu, Chesterfield, MO (US); Jianmin Zhao, St. Louis, MO (US); Albert Iandolino, Davis, CA (US); Monica Ravanello, Fairfield, CA (US); Kraig Brustad, Stonington, CT (US); Paul Chomet, Mystic, CT (US); Julie Davis, North Stonington, CT (US); Virginia Ursin, Pawcatuck, CT (US); T. Ambika, Bangalore (IN); Shobha Char, Bangalore (IN); Savitha Madappa, Bangalore (IN); Dhanalakshmi Ramachandra, Bangalore (IN); S. Sangeetha, Bangalore (IN); K. R. Vidya, Bangalore (IN); K. Vijayalakshmi, Bangalore (IN); Todd Dezwaan, Apex, NC (US)

(73) Assignee: Monsanto Technology LLC, St. Louis, MO (US)

(21) Appl. No.: 13/566,369

(22) Filed: Aug. 3, 2012

Related U.S. Application Data
(63) Continuation of application No. 12/322,120, filed on Jan. 29, 2009, now abandoned.
(60) Provisional application No. 61/024,667, filed on Jan. 30, 2008.
(60) Provisional application No. 61/035,103, filed on Mar. 10, 2008.
(60) Provisional application No. 61/047,017, filed on Apr. 22, 2008.
(60) Provisional application No. 61/048,708, filed on Apr. 29, 2008.
(60) Provisional application No. 61/078,027, filed on Jul. 3, 2008.

(51) Int. Cl. C12N 15/82

(52) U.S. Cl. CPC C12N 15/8273; C12N 15/8247; C12N 15/8251
USPC 800/260; 435/6.1; 435/418; 435/419; 435/320.1; 536/23.6; 800/298; 800/306; 800/312; 800/314; 800/320; 800/320.1; 800/320.2

(57) ABSTRACT

This invention provides transgenic plant cells with recombinant DNA for expression of proteins that are useful for imparting enhanced agronomic trait(s) to transgenic crop plants. This invention also provides transgenic plants and progeny seed comprising the transgenic plant cells where the plants are selected for having an enhanced trait selected from the group of traits consisting of enhanced water use efficiency, enhanced cold tolerance, increased yield, enhanced nitrogen use efficiency, enhanced seed protein and enhanced seed oil. Also disclosed are methods for manufacturing transgenic seed and plants with enhanced traits.

### CROSS REFERENCE TO RELATED APPLICATIONS

This application is a continuation of and claims the benefit of priority under 35 U.S.C. §120 to U.S. patent application Ser. No. 12/322,120, entitled “TRANSGENIC PLANTS WITH ENHANCED AGRONOMIC TRAITS,” filed on Jan. 29, 2009, which claims the benefit of priority under 35 USC §119(e) to U.S. provisional application Ser. Nos. 61/024,667, filed Jan. 30, 2008, 61/035,103, filed Mar. 10, 2008, 61/047,017, filed Apr. 22, 2008, 61/048,708, filed Apr. 29, 2008, and 61/078,027, filed Jul. 3, 2008, each of which is incorporated herein by reference in its entirety.

### INCORPORATION OF SEQUENCE LISTING

Two copies of the sequence listing (Copy 1 and Copy 2) and a computer readable form (CRF) of the sequence listing, all non CD-Rs, each containing the text file named “55591_B_seq_listing.txt”, which is 74,407,936 bytes (measured in MS-WINDOWS), were created on Jan. 27, 2009, and filed Jan. 29, 2009, for the application accorded Ser. No. 12/322,120; and the sequence listing file “55591_B_seq_listing corrected.txt” which is 74,411,868 bytes and was created on Mar. 16, 2010 as well as the material on replacement CDs (Copy #1 Replacement and Copy #2 Replacement) named “Sequence,” file name “3126019US1.txt,” file size 74,411,868 bytes created on Aug. 9, 2010, and filed Aug. 9, 2010, for the application accorded Ser. No. 12/322,120, are incorporated herein by reference.

### Incorporation of Large Table

Two copies of a large table (Copy 1 and Copy 2) containing a folder “pfamdir” on CD-Rs are incorporated herein by reference in their entirety. Folder “pfamdir” contains 103 Pfam Hidden Markov Models. The CD-Rs were created on Jan. 27, 2009, having a total size of 7,665,424 bytes (measured in MS-WINDOWS), and filed on Jan. 29, 2009, for the application accorded Ser. No. 12/322,120 and incorporated herein by reference.

### FIELD OF THE INVENTION

Disclosed herein are recombinant DNA useful for providing enhanced traits to transgenic plants, seeds, pollen, plant cells and plant nuclei of such transgenic plants, methods of making and using such recombinant DNA, plants, seeds, pollen, plant cells and plant nuclei. Also disclosed are methods of producing hybrid corn seed comprising such recombinant DNA. All genetic resources disclosed herein were directly obtained from sources that are currently common to the United States; the ancestral sources of each specific genetic material is unknown.

### SUMMARY OF THE INVENTION

An aspect of this invention provides recombinant DNA constructs comprising polynucleotides characterized by an encoded protein having amino acids representing a protein family domain module as described in Table 10. Another aspect of this invention provides recombinant DNA constructs comprising polynucleotides characterized by an encoded protein with an amino acid sequence that is at least 90% identical to a corresponding consensus sequence defined in table 8. Yet another aspect of this invention provides recombinant DNA constructs comprising polynucleotides characterized by reference to SEQ ID NO:1-121 and the cognate proteins with amino acid sequences having reference to SEQ ID NO:122-242. The recombinant DNA constructs are useful for providing enhanced traits when stably integrated into the chromosomes and expressed in the nuclei of transgenic plants cells. In some aspects of the invention the recombinant DNA constructs, when expressed in a plant cell, provide for expression of cognate proteins. In those aspects of the invention, the recombinant DNA constructs for expressing cognate proteins are characterized by cognate amino acid sequences having a sequence selected from SEQ ID NOs: 123-140, 145-151, 153-173, 175-187, 189, 191-202, 204-207, 209-238, and 242; having at least 95% identity over at least 95% of the length of a sequence selected from the group consisting of SEQ ID NOs: 123-140, 145-151, 153-173, 175-187, 189, 191-202, 204-207, 209-238, and 242 or that are homologous to a sequence selected from the group consisting of SEQ ID NOs: 123-140, 145-151, 153-173, 175-187, 189, 191-202, 204-207, 209-238, and 242.

In other aspects of the invention the recombinant DNA constructs provide for suppression of a native protein. In those other aspects of the invention the recombinant DNA constructs are characterized as being constructed with sense-oriented and anti-sense-oriented polynucleotides, e.g. polynucleotides derived from genes having SEQ ID NOs: 1, 20-23, 31, 53, 67, 69, 82, 87, and 118-120 or homologous genes. When the recombinant DNA construct is expressed in corn plants, the endogenous protein is a corn protein with an amino acid sequence of SEQ ID NO:141-144, 152, 174, 190, 203, 208, or 239-240 or the corn homolog of SEQ ID NOs:122, 188, or 241; when the recombinant DNA construct is expressed in soybean plants, the endogenous protein is a soybean protein with an amino acid sequence of SEQ ID NO: 122, 188, or 241 or is a soybean homolog of SEQ ID NOs: 141-144, 152, 174, 190, 203, 208, or 239-240; and when the recombinant DNA construct is expressed in a plant other than a corn or a soybean plant, the endogenous protein is the other plant's endogenous protein that has an amino acid sequence homologous to SEQ ID NO: 122, 141-144, 152, 174, 188, 190, 203, 208, or 239-241.

In practical aspects of this invention the recombinant DNA constructs of the invention are stably integrated into the chromosome of a plant cell nucleus.

This invention also provides transgenic plant cells comprising the stably integrated recombinant DNA constructs of the invention, transgenic plants and seeds comprising a plurality of such transgenic plant cells and transgenic pollen of such plants. Such transgenic plants are selected from a population of transgenic plants regenerated from plant cells transformed with recombinant DNA constructs by screening transgenic plants for an enhanced trait as compared to control plants. The enhanced trait is one or more of enhanced water use efficiency, enhanced cold tolerance, increased yield, enhanced nitrogen use efficiency, enhanced seed protein and enhanced seed oil.

In another aspect of the invention the plant cells, plants, seeds, and pollen further comprise DNA expressing a protein that provides tolerance from exposure to an herbicide applied at levels that are lethal to a wild type plant cell.

This invention also provides methods for manufacturing non-natural, transgenic seed that can be used to produce a crop of transgenic plants with an enhanced trait resulting from expression of a stably-integrated recombinant DNA construct. More specifically, the method comprises (a) screening a population of plants for an enhanced trait and a recombinant DNA construct, where individual plants in the population can exhibit the trait at a level less than, essentially the same as or greater than the level that the trait is exhibited in control plants, (b) selecting from the population one or more plants that exhibit the trait at a level greater than the level that said trait is exhibited in control plants, (c) collecting seed from a selected plant, (d) verifying that the recombinant DNA is stably integrated in said selected plants, (e) analyzing tissue of a selected plant to determine the production or suppression of a protein having the function of a protein encoded by nucleotides in a sequence of one of SEQ ID NOs:1-121. In one aspect of the invention, the plants in the population further comprise DNA expressing a protein that provides tolerance to exposure to a herbicide applied at levels that are lethal to wild type plant cells and the selecting is affected by treating the population with the herbicide, e.g. a glyphosate, dicamba, or glufosinate compound. In another aspect of the invention the plants are selected by identifying plants with the enhanced trait. The methods are especially useful for manufacturing corn, soybean, cotton, canola, alfalfa, wheat, rice, sugarcane or sugar beet seed.

Another aspect of the invention provides a method of producing hybrid corn seed comprising acquiring hybrid corn seed from a herbicide tolerant corn plant which also has stably-integrated, recombinant DNA construct comprising a promoter that is (a) functional in plant cells and (b) is operably linked to DNA that encodes or suppresses a protein having the function of a protein encoded by nucleotides in a sequence of one of SEQ ID NOs:1-121. The methods further comprise producing corn plants from said hybrid corn seed, wherein a fraction of the plants produced from said hybrid corn seed is homozygous for said recombinant DNA, a fraction of the plants produced from said hybrid corn seed is hemizygous for said recombinant DNA, and a fraction of the plants produced from said hybrid corn seed has none of said recombinant DNA; selecting corn plants which are homozygous and hemizygous for said recombinant DNA by treating with an herbicide; collecting seed from herbicide-treated-surviving corn plants and planting said seed to produce further progeny corn plants; repeating the selecting and collecting steps at least once to produce an inbred corn line; and crossing the inbred corn line with a second corn line to produce hybrid seed.

Another aspect of the invention provides a method of selecting a plant comprising plant cells of the invention by using an immunoreactive antibody to detect the presence or absence of protein expressed or suppressed by recombinant DNA in seed or plant tissue. Yet another aspect of the invention provides anti-counterfeit milled seed having, as an indication of origin, plant cells of this invention.

Still other aspects of this invention relate to transgenic plants with enhanced water use efficiency or enhanced nitrogen use efficiency. For instance, this invention provides methods of growing a corn, cotton, soybean, or canola crop without irrigation water comprising planting seed having plant cells of the invention which are selected for enhanced water use efficiency. Alternatively methods comprise applying reduced irrigation water, e.g. providing up to 300 millimeters of ground water during the production of a corn crop. This invention also provides methods of growing a corn, cotton, soybean or canola crop without added nitrogen fertilizer comprising planting seed having plant cells of the invention which are selected for enhanced nitrogen use efficiency.

Another aspect of the invention provides a mixture comprising plants cells and an antibody to a protein produced in the cells where the protein has an amino acid sequence that has at least 95% identity over at least 95% of the length of a reference sequence selected from the group consisting of SEQ ID NO: 122-242 when the sequence is aligned to the reference sequence.

### BRIEF DESCRIPTION OF THE DRAWINGS

FIGS. 1-4 are plasmid maps.

### DETAILED DESCRIPTION OF THE INVENTION

In the attached sequence listing:

SEQ ID NO:1-121 are nucleotide sequences of the coding strand of DNA for “genes” used in the recombinant DNA imparting an enhanced trait in plant cells, i.e. each represents a coding sequence for a protein;

SEQ ID NO: 122-242 are amino acid sequences of the cognate protein of the “genes” with nucleotide coding sequences 1-121;

SEQ ID NO: 243-17376 are amino acid sequences of homologous proteins;

SEQ ID NO: 17377 is a nucleotide sequence of a base plasmid vector useful for corn transformation;

SEQ ID NO: 17378 is a nucleotide sequence of a base plasmid vector useful for soybean and canola transformation;

SEQ ID NO: 17379 is a nucleotide sequence of a base plasmid vector useful for cotton transformation;

SEQ ID NO: 17380 is a nucleotide sequence of a base plasmid vector useful for co-transformation to produce gene stacks in corn;

SEQ ID NO: 17381-17402 are consensus sequences.

Table 8 lists the protein SEQ ID NOs and their corresponding consensus SEQ ID NOs.

As used herein a “plant cell” means a plant cell that is transformed with stably-integrated, non-natural, recombinant DNA, e.g. by Agrobacterium-mediated transformation or by bombardment using microparticles coated with recombinant DNA or other means. A plant cell of this invention can be an originally-transformed plant cell that exists as a microorganism or as a progeny plant cell that is regenerated into differentiated tissue, e.g. into a transgenic plant with stably-integrated, non-natural recombinant DNA, or seed or pollen derived from a progeny transgenic plant.

As used herein a “transgenic plant” means a plant whose genome has been altered by the stable integration of recombinant DNA. A transgenic plant includes a plant regenerated from an originally-transformed plant cell and progeny transgenic plants from later generations or crosses of a transformed plant.

As used herein “recombinant DNA” means DNA which has been a genetically engineered and constructed outside of a cell including DNA containing naturally occurring DNA or cDNA or synthetic DNA.

As used herein “consensus sequence” means an artificial sequence of amino acids in a conserved region of an alignment of amino acid sequences of homologous proteins, e.g. as determined by a CLUSTALW alignment of amino acid sequence of homolog proteins.

As used herein a “homolog” means a protein in a group of proteins that perform the same biological function, e.g. proteins that belong to the same Pfam protein family and that provide a common enhanced trait in transgenic plants of this invention. Homologs are expressed by homologous genes. With reference to homologous genes, homologs include orthologs, i.e. genes expressed in different species that evolved from a common ancestral genes by speciation and encode proteins retain the same function, but do not include paralogs, i.e. genes that are related by duplication but have evolved to encode proteins with different functions. Homologous genes include naturally occurring alleles and artificially-created variants. Degeneracy of the genetic code provides the possibility to substitute at least one base of the protein encoding sequence of a gene with a different base without causing the amino acid sequence of the polypeptide produced from the gene to be changed. When optimally aligned, homolog proteins have at least 60% identity, more preferably about 65% or higher, more preferably about 70% or higher, more preferably at least 75%, more preferably at least 80%, more preferably at least 85%, more preferably at least 90% identity, more preferably at least 95, 96, 97, 98, or 99% identity over the full length of a protein identified as being associated with imparting an enhanced trait when expressed in plant cells. In one aspect of the invention homolog proteins have an amino acid sequence that has at least 90% identity to a consensus amino acid sequence of proteins and homologs disclosed herein.

Homologs are identified by comparison of amino acid sequence, e.g. manually or by use of a computer-based tool using known homology-based search algorithms such as the suite of BLAST programs available from NCBI. A local sequence alignment program, e.g. BLAST, can be used to search a database of sequences to find similar sequences, and the summary Expectation value (E-value) used to measure the sequence base similarity. Because a protein hit with the best E-value for a particular organism may not necessarily be an ortholog, i.e. have the same function, or be the only ortholog, a reciprocal query is used to filter hit sequences with significant E-values for ortholog identification. The reciprocal query entails search of the significant hits against a database of amino acid sequences from the base organism that are similar to the sequence of the query protein. A hit can be identified as an ortholog, when the reciprocal query's best hit is the query protein itself or a protein encoded by a duplicated gene after speciation. A further aspect of the homologs encoded by DNA useful in the transgenic plants of the invention are those proteins that differ from a disclosed protein as the result of deletion or insertion of one or more amino acids in a native sequence.

Percent identity describes the extent to which the sequences of DNA or protein segments are invariant in an alignment of sequences, for example nucleotide sequences or amino acid sequences. An alignment of sequences is created by manually aligning two sequences, e.g. a stated sequence, as provided herein, as a reference, and another sequence, to produce the highest number of matching elements, e.g. individual nucleotides or amino acids, while allowing for the introduction of gaps into either sequence. An “identity fraction” for a sequence aligned with a reference sequence is the number of matching elements, divided by the full length of the reference sequence, not including gaps introduced by the alignment process into the reference sequence. “Percent identity” (“% identity”) as used herein is the identity fraction times 100.

“Pfam” is a large collection of multiple sequence alignments and hidden Markov models covering many common protein families, e.g. Pfam version 19.0 (December 2005) contains alignments and models for 8183 protein families and is based on the Swissprot 47.0 and SP-TrEMBL 30.0 protein sequence databases. See S. R. Eddy, “Profile Hidden Markov Models”, Bioinformatics 14:755-763, 1998. The Pfam database is currently maintained and updated by the Pfam Consortium. The alignments represent some evolutionary conserved structure that has implications for the protein's function. Profile hidden Markov models (profile HMMs) built from the protein family alignments are useful for automatically recognizing that a new protein belongs to an existing protein family even if the homology by alignment appears to be low.

Protein domains are identified by querying the amino acid sequence of a protein against Hidden Markov Models which characterize protein family domains (“Pfam domains”) using HMMER software, which is available from the Pfam Consortium. The HMMER software is also disclosed in patent application publication US 2008/0148432 A1 incorporated herein by reference. A protein domain meeting the gathering cutoff for the alignment of a particular Pfam domain is considered to contain the Pfam domain.

A “Pfam domain module” is a representation of Pfam domains in a protein, in order from N terminus to C terminus. In a Pfam domain module individual Pfam domains are separated by double colons “::”. The order and copy number of the Pfam domains from N to C terminus are attributes of a Pfam domain module. Although the copy number of repetitive domains is important, varying copy number often enables a similar function. Thus, a Pfam domain module with multiple copies of a domain should define an equivalent Pfam domain module with variance in the number of multiple copies. A Pfam domain module is not specific for distance between adjacent domains, but contemplates natural distances and variations in distance that provide equivalent function. The Pfam database contains both narrowly- and broadly-defined domains, leading to identification of overlapping domains on some proteins. A Pfam domain module is characterized by non-overlapping domains. Where there is overlap, the domain having a function that is more closely associated with the function of the protein (based on the E value of the Pfam match) is selected.

Once one DNA is identified as encoding a protein which imparts an enhanced trait when expressed in transgenic plants, other DNA encoding proteins with the same Pfam domain module are identified by querying the amino acid sequence of protein encoded by candidate DNA against the Hidden Markov Models which characterizes the Pfam domains using HMMER software. Candidate proteins meeting the same Pfam domain module are in the protein family and have cognate DNA that is useful in constructing recombinant DNA for the use in the plant cells of this invention. Hidden Markov Model databases for use with HMMER software in identifying DNA expressing protein with a common Pfam domain module for recombinant DNA in the plant cells of this invention are included in the large table incorporated into this application.

The HMMER software and Pfam databases (version 19.0) were used to identify known domains in the proteins corresponding to amino acid sequence of SEQ ID NOs: 123-132, 134-135, 137, 139-140, 142, 145-152, 154, 156-172, 175-181, 183-196, 198-202, 204-205, 207, 209-211, 213, 216, 218-228, 230-233, 235, 238-239, 241-242. All DNA encoding proteins that have scores higher than the gathering cutoff disclosed in Table 11 by Pfam analysis disclosed herein can be used in recombinant DNA of the plant cells of this invention, e.g. for selecting transgenic plants having enhanced agronomic traits. The relevant Pfarns modules for use in this invention, as more specifically disclosed below, are 14-3-3, 20G-FeII_Oxy, AhpC-TSA::1cysPrx_C, AP2, B3::Auxin_resp::AUX_IAA, Brix, CBFB_NFYA, CBFD_NFYB_HMF, Cellulose_synt, Copine, DnaJ, DUF231, DUF260, DUF296, DUF761, DUF778, E2F_TDP, efhand, FAR1::FAR1, F-box, Fer2::Fer2_{—}2::FAD_binding_{—}5::CO_deh_flav_C::Ald_Xan_dh_C::Ald_Xan_dh_C2, GATase_{—}2::Glu_syn_centrak:Glu_synthase::GXGXG, Gln-synt_N::Gln-synt_C, Globin, Glu_synthase, Glyco_hydro_{—}18, Glyco_transf 20::Trehalose_PPase, Glycos_transf_{—}1, GMC_oxred_N, GSHPx, HEAT::HEAT::FAT::Rapamycin_bind::PI3_PI4_kinase::FATC, HLH, HSP20, HSP70, LRRNT_{—}2::LRR_{—}1::LRR_{—}1::LRR_{—}1::Pkinase_Tyr, Mannitol_dh::Mannitol_dh_C, Na_H_Exchanger, NAPRTase, Nucleoside_tran, PALP, PAS_{—}2::GAF::Phytochrome::PAS, PAS_{—}2::GAF::Phytochrome::PAS::PAS::H isKA::HATPase_c, PAS_{—}3::PAS::Pkinase, PDZ::Peptidase_S41, Peptidase_S10, Peptidase_S51, PPR::PPR::PPR::PPR::PPR::PPR::PPR::PPR::PPR::PPR::PPR, PTR2, RAMP4, Response_reg, RimK::Mur_ligase_M::Mur_ligase_C, RNase_PH_C, SAM_{—}1::DRMBL, SAP::PHD::zf-MIZ, SAP::zf-MIZ, SNF2—N::Helicase_C, Spermine_synth, SRF-TF, SSF, Synaptobrevin, TP6A_N, TrkH, UDPGP, XRN_N::zf-CCHC, zf-A20::zf-AN1, and zf-B_box, for which databases are included in the appended computer listing.

As used herein “promoter” means regulatory DNA for initializing transcription. A “plant promoter” is a promoter capable of initiating transcription in plant cells whether or not its origin is a plant cell, e.g. is it well known that Agrobacterium promoters are functional in plant cells. Thus, plant promoters include promoter DNA obtained from plants, plant viruses and bacteria such as Agrobacterium and Bradyrhizobium bacteria. Examples of promoters under developmental control include promoters that preferentially initiate transcription in certain tissues, such as leaves, roots, or seeds. Such promoters are referred to as “tissue preferred”. Promoters that initiate transcription only in certain tissues are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions, or certain chemicals, or the presence of light. Tissue specific, tissue preferred, cell type specific, and inducible promoters constitute the class of “non-constitutive” promoters. A “constitutive” promoter is a promoter which is active under most conditions.

As used herein “operably linked” means the association of two or more DNA fragments in a recombinant DNA construct so that the function of one, e.g. protein-encoding DNA, is controlled by the other, e.g. a promoter.

As used herein “expressed” means produced, e.g. a protein is expressed in a plant cell when its cognate DNA is transcribed to mRNA that is translated to the protein.

As used herein “suppressed” means decreased, e.g. a protein is suppressed in a plant cell when there is a decrease in the amount and/or activity of the protein in the plant cell. The presence or activity of the protein can be decreased by any amount up to and including a total loss of protein expression and/or activity.

As used herein a “control plant” means a plant that does not contain the recombinant DNA that imparts an enhanced trait. A control plant is used to identify and select a transgenic plant that has an enhanced trait. A suitable control plant can be a non-transgenic plant of the parental line used to generate a transgenic plant, i.e. devoid of recombinant DNA. A suitable control plant may in some cases be a progeny of a hemizygous transgenic plant line that does not contain the recombinant DNA, known as a negative segregant.

As used herein an “enhanced trait” means a characteristic of a transgenic plant that includes, but is not limited to, an enhance agronomic trait characterized by enhanced plant morphology, physiology, growth and development, yield, nutritional enhancement, disease or pest resistance, or environmental or chemical tolerance. In more specific aspects of this invention enhanced trait is selected from group of enhanced traits consisting of enhanced water use efficiency, enhanced cold tolerance, increased yield, enhanced nitrogen use efficiency, enhanced seed protein and enhanced seed oil. In an important aspect of the invention the enhanced trait is enhanced yield including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), ear number, seed number per ear, seed size, composition of seed (starch, oil, protein) and characteristics of seed fill.

Increased yield of a transgenic plant of the present invention can be measured in a number of ways, including test weight, seed number per plant, seed weight, seed number per unit area (i.e. seeds, or weight of seeds, per acre), bushels per acre, tons per acre, or kilo per hectare. For example, corn yield may be measured as production of shelled corn kernels per unit of production area, for example in bushels per acre or metric tons per hectare, often reported on a moisture adjusted basis, for example at 15.5 percent moisture. Increased yield may result from improved utilization of key biochemical compounds, such as nitrogen, phosphorous and carbohydrate, or from improved responses to environmental stresses, such as cold, heat, drought, salt, and attack by pests or pathogens. Recombinant DNA used in this invention can also be used to provide plants having improved growth and development, and ultimately increased yield, as the result of modified expression of plant growth regulators or modification of cell cycle or photosynthesis pathways. Also of interest is the generation of transgenic plants that demonstrate enhanced yield with respect to a seed component that may or may not correspond to an increase in overall plant yield. Such properties include enhancements in seed oil, seed molecules such as protein and starch, oil components as may be manifest by an alterations in the ratios of seed components.

Recombinant DNA constructs are assembled using methods well known to persons of ordinary skill in the art and typically comprise a promoter operably linked to DNA, the expression of which provides the enhanced agronomic trait. Other construct components may include additional regulatory elements, such as 5′ leaders and introns for enhancing transcription, 3′ untranslated regions (such as polyadenylation signals and sites), DNA for transit or signal peptides.

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasmids of Agrobacterium tumefaciens and the CaMV35S promoters from the cauliflower mosaic virus as disclosed in U.S. Pat. Nos. 5,164,316 and 5,322,938. Useful promoters derived from plant genes are found in U.S. Pat. No. 5,641,876 which discloses a rice actin promoter, U.S. Pat. No. 7,151,204 which discloses a maize chloroplast aldolase promoter and a maize aldolase (FDA) promoter, and US Patent Application Publication 2003/0131377 A1 which discloses a maize nicotianamine synthase promoter. These and numerous other promoters that function in plant cells are known to those skilled in the art and available for use in recombinant polynucleotides of the present invention to provide for expression of desired genes in transgenic plant cells.

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see U.S. Pat. No. 5,641,876) and rice actin 2 genes, the maize alcohol dehydrogenase gene intron, the maize heat shock protein 70 gene intron (U.S. Pat. No. 5,593,874) and the maize shrunken 1 gene. See also US Patent Application Publication 2002/0192813A1 which discloses 5′, 3′ and intron elements useful in the design of effective plant expression vectors.

In other aspects of the invention, sufficient expression in plant seed tissues is desired to affect improvements in seed composition. Exemplary promoters for use for seed composition modification include promoters from seed genes such as napin as disclosed in U.S. Pat. No. 5,420,034, maize L3 oleosin as disclosed in U.S. Pat. No. 6,433,252), zein Z27 as disclosed by Russell et al. (1997) Transgenic Res. 6(2):157-166), globulin 1 as disclosed by Belanger et al (1991) Genetics 129:863-872), glutelin 1 as disclosed by Russell (1997) supra), and peroxiredoxin antioxidant (Per1) as disclosed by Stacy et al. (1996) Plant Mol Biol. 31(6):1205-1216.

Recombinant DNA constructs useful in this invention will also generally include a 3′ element that typically contains a polyadenylation signal and site. Well-known 3′ elements include those from Agrobacterium tumefaciens genes such as nos 3′, tml 3′, tmr 3′, tms 3′, ocs 3′, tr7 3′, for example disclosed in U.S. Pat. No. 6,090,627; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene, a rice lactate dehydrogenase gene and a rice beta-tubulin gene, all of which are disclosed in US Patent Application Publication 2002/0192813 A1; and the pea (Pisum sativum) ribulose biphosphate carboxylase gene (rbs 3′), and 3′ elements from the genes within the host plant.

Constructs and vectors may also include a transit peptide for targeting of a gene to a plant organelle, particularly to a chloroplast, leucoplast or other plastid organelle. For descriptions of the use of chloroplast transit peptides see U.S. Pat. No. 5,188,642 and U.S. Pat. No. 5,728,925. For description of the transit peptide region of an Arabidopsis EPSPS gene useful in the present invention, see Klee, H. J. et al (MGG (1987) 210:437-442).

Recombinant DNA constructs for gene suppression can be designed for any of a number the well-known methods for suppressing transcription of a gene, the accumulation of the mRNA corresponding to that gene or preventing translation of the transcript into protein. Posttranscriptional gene suppression can be practically effected by transcription of RNA that forms double-stranded RNA (dsRNA) having homology to mRNA produced from a gene targeted for suppression.

Gene suppression can also be achieved by insertion mutations created by transposable elements may also prevent gene function. For example, in many dicot plants, transformation with the T-DNA of Agrobacterium may be readily achieved and large numbers of transformants can be rapidly obtained. Also, some species have lines with active transposable elements that can efficiently be used for the generation of large numbers of insertion mutations, while some other species lack such options. Mutant plants produced by Agrobacterium or transposon mutagenesis and having altered expression of a polypeptide of interest can be identified using the polynucleotides of the present invention. For example, a large population of mutated plants may be screened with polynucleotides encoding the polypeptide of interest to detect mutated plants having an insertion in the gene encoding the polypeptide of interest.

Transgenic plants may comprise a stack of one or more polynucleotides disclosed herein resulting in the production or suppression of multiple polypeptide sequences, Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene, and co-transformation of genes into a single plant cell. Co-transformation of genes can be carried out using single transformation vectors comprising multiple genes or genes carried separately on multiple vectors.

Transgenic plants comprising or derived from plant cells of this invention transformed with recombinant DNA can be further enhanced with stacked traits, e.g. a crop plant having an enhanced trait resulting from expression of DNA disclosed herein in combination with herbicide and/or pest resistance traits. For example, genes of the current invention can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects. Herbicides for which transgenic plant tolerance has been demonstrated and the method of the present invention can be applied include, but are not limited to, glyphosate, dicamba, glufosinate, sulfonylurea, bromoxynil and norflurazon herbicides. Polynucleotide molecules encoding proteins involved in herbicide tolerance are well-known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in US Patent Application Publication 2003/0083480 A1 also for imparting glyphosate tolerance; dicamba monooxygenase disclosed in US Patent Application Publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al, (1993)Plant J. 4:833-840 and in Misawa et al, (1994) Plant J. 6; 481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in US Patent Application Publication 2003/010609 A1 for imparting N-amino methyl phosphonic acid tolerance; polynucleotide molecules disclosed in U.S. Pat. No. 6,107,549 for impartinig pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and US Patent Application Publication 2002/0112260. Molecules and methods for imparting insect/nematode/virus resistance are disclosed in U.S. Pat. Nos. 5,250,515; 5,880,275; 6,506,599; 5,986,175 and US Patent Application Publication 2003/0150017 A1.

### Plant Cell Transformation Methods

Numerous methods for transforming chromosomes in a plant cell nucleus with recombinant DNA are known in the art and are used in methods of preparing a transgenic plant cell nucleus cell, and plant. Two effective methods for such transformation are Agrobacterium-mediated transformation and microprojectile bombardment. Microprojectile bombardment methods are illustrated in U.S. Pat. Nos. 5,015,580 (soybean); 5,550,318 (corn); 5,538,880 (corn); 5,914,451 (soybean); 6,160,208 (corn); 6,399,861 (corn); 6,153,812 (wheat) and 6,365,807 (rice) and Agrobacterium-mediated transformation is described in U.S. Pat. Nos. 5,159,135 (cotton); 5,824,877 (soybean); 5,463,174 (canola); 5,591,616 (corn); 5,846,797 (cotton); 6,384,301 (soybean), 7,026,528 (wheat) and 6,329,571 (rice), US Patent Application Publication 2004/0087030 A1 (cotton), and US Patent Application Publication 2001/0042257 A1 (sugar beet), all of which are incorporated herein by reference for enabling the production of transgenic plants. Transformation of plant material is practiced in tissue culture on a nutrient media, i.e. a mixture of nutrients that will allow cells to grow in vitro. Recipient cell targets include, but are not limited to, meristem cells, hypocotyls, calli, immature embryos and gametic cells such as microspores, pollen, sperm and egg cells. Callus may be initiated from tissue sources including, but not limited to, immature embryos, hypocotyls, seedling apical meristems, microspores and the like. Cells containing a transgenic nucleus are grown into transgenic plants.

In addition to direct transformation of a plant material with a recombinant DNA, a transgenic plant cell nucleus can be prepared by crossing a first plant having cells with a transgenic nucleus with recombinant DNA with a second plant lacking the transgenic nucleus. For example, recombinant DNA can be introduced into a nucleus from a first plant line that is amenable to transformation to transgenic nucleus in cells that are grown into a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, e.g. marker identification by analysis for recombinant DNA or, in the case where a selectable marker is linked to the recombinant, by application of the selecting agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as one original transgenic parental line but for the recombinant DNA of the other transgenic parental line

In the practice of transformation DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or a herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO₂, and 25-250 microeinsteins m⁻² s⁻¹ of light, prior to transfer to a greenhouse or growth chamber for maturation. Plants are regenerated from about 6 weeks to 10 months after a transformant is identified, depending on the initial tissue, and plant species. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

### Transgenic Plants and Seeds

Transgenic plants derived from transgenic plant cells having a transgenic nucleus of this invention are grown to generate transgenic plants having an enhanced trait as compared to a control plant and produce transgenic seed and haploid pollen of this invention. Such plants with enhanced traits are identified by selection of transformed plants or progeny seed for the enhanced trait. For efficiency a selection method is designed to evaluate multiple transgenic plants (events) comprising the recombinant DNA, for example multiple plants from 2 to 20 or more transgenic events. Transgenic plants grown from transgenic seed provided herein demonstrate improved agronomic traits that contribute to increased yield or other trait that provides increased plant value, including, for example, improved seed quality. Of particular interest are plants having enhanced water use efficiency, enhanced cold tolerance, increased yield, enhanced nitrogen use efficiency, enhanced seed protein and enhanced seed oil. Table 1 provides a list of protein encoding DNA (“genes”) that are useful as recombinant DNA for production of transgenic plants with enhanced agronomic trait, the elements of Table 1 are described by reference to:

“PEP SEQ ID NO” identifies an amino acid sequence from SEQ ID NO: 122 to 242.
“NUC SEQ ID NO” identifies a DNA sequence from SEQ ID NO:1 to 121.
“Gene ID” refers to an arbitrary identifier.
“Gene Name” denotes a common name for the protein encoded by the recombinant DNA preceded by the abbreviated genus and species as fully defined in the sequence listing. The + or − preceding the gene name indicates whether the protein is expressed (+) or suppressed (−) in plants to provide an enhanced trait.
“Annotation” refers to a description of the top hit protein obtained from an amino acid sequence query of each PEP SEQ ID NO to GENBANK database of the National Center for Biotechnology Information (ncbi).

TABLE 1

| NUC | PEP | | | |
|---|---|---|---|---|
| SEQ ID | SEQ ID | | | |
| NO | NO | Gene ID | Gene Name | Annotation |
| 1 | 122 | Mnom000132-Mnom000134 | −Gm.GW2 | emb\|CAN66658.1\|hypothetical protein [Vitis |
| | | | | vinifera] |
| 2 | 123 | Mnom000202 | +At.CGPG5239 | gb\|ABM06011.1\|At1g67800 [Arabidopsis |
| | | | | thaliana] |
| 3 | 124 | Mnom000215-Mnom000216 | +Ta.NF-YA1 | ref\|NP_001048285.1\|Os02g0776400 [Oryza |
| | | | | sativa (japonica cultivar-group)] |
| 4 | 125 | Mnom000219-Mnom000220 | +Zm.NF- | ref\|NP_001060230.1\|Os07g0606600 [Oryza |
| | | | YB3/7/8 | sativa (japonica cultivar-group)] |
| 5 | 126 | Mnom000256-Mnom000257 | +Bs.GOGATII | ref\|NP_388541.1\|hypothetical protein BSU06590 |
| | | | | [Bacillus subtilis subsp. subtilis str. 168] |
| 6 | 127 | Mnom000260-Mnom000261 | +Zm.TOR | gb\|AAW78347.1\|target of rapamycin [Zea mays] |
| 7 | 128 | Mnom000262 | +Zm.APO1 | dbj\|BAD45387.1\|putative stamina pistilloidia |
| | | | | [Oryza sativa (japonica |
| | | | | cultivar-group)] |
| 8 | 129 | Mnom000324 | +Gm.PTI4-3 | AF245119_1 AP2-related transcription factor |
| | | | MRT3847_21399C | [Mesembryanthemum crystallinum] |
| 9 | 130 | Mnom000372 | +Sc.SIZ1 | gb\|AAB64849.1\|Ydr409wp; CAI: 0.12 |
| | | | | [Saccharomyces cerevisiae] |
| 10 | 131 | Mnom000412-Mnom000413 | +Os.GF14h | gb\|ABA94733.1\|14-3-3, putative, expressed |
| | | | | [Oryza sativa (japonica |
| | | | | cultivar-group)] |
| 11 | 132 | Mnom000444 | +Lb. | dbj\|BAA12858.1\|ferredoxin-dependent glutamate |
| | | | Ferredoxin- | synthase [Leptolyngbya boryana] |
| | | | dependent | |
| | | | glutamate | |
| | | | synthase | |
| 12 | 133 | Mnom000451 | +Gm.G3083-like 2 | gb\|EAY72627.1\|hypothetical protein |
| | | | | OsI_000474 [Oryza sativa (indica cultivar- |
| | | | | group)] |
| 13 | 134 | Mnom000471 | +Sc.TPS-TPP | ref\|NP_010359.1\|Phosphatase subunit of the |
| | | | | trehalose-6-phosphate synthase/phosphatase |
| | | | | complex, which synthesizes the |
| | | | | storage carbohydrate trehalose; expression is |
| | | | | induced by stress conditions and repressed by the |
| | | | | Ras-cAMP pathway [Saccharomyces cerevisiae] |
| 14 | 135 | Mnom000480 | +Zm.A1ZM027162_at_OSJ | >gb\|EAY93657.1\|hypothetical protein |
| | | | NBa0020I02.17 | OsI_014890 [Oryza sativa (indica cultivar- |
| | | | protein | group)] |
| 15 | 136 | Mnom000481 | +Zm.A1ZM003077_s_at_Os | gb\|EAY78482.1\|hypothetical protein |
| | | | 10g0415800 | OsI_032441 [Oryza sativa (indica |
| | | | protein | cultivar-group)] |
| 16 | 137 | Mnom000485 | +Zm.A1ZM058479_at_FAR1 | gb\|ABF95032.1\|FAR1 family protein, expressed |
| | | | family | [Oryza sativa (japonica cultivar-group)] |
| | | | protein, | |
| | | | expressed | |
| 17 | 138 | Mnom000486 | +Zm.A1ZM016696_x_at_Hypothetical | gb\|EAY85998.1\|hypothetical protein |
| | | | protein | OsI_007231 [Oryza sativa (indica cultivar- |
| | | | OSJNBa0052M16.13 | group)] |
| 18 | 139 | Mnom000490 | +Zm.A1ZM057199_s_at_Peptide | >gb\|EAY89170.1\|hypothetical protein |
| | | | transporter | OsI_010403 [Oryza sativa (indica cultivar- |
| | | | PTR2, | group)] |
| | | | putative, | |
| | | | expressed | |
| 19 | 140 | Mnom000491 | +Zm.A1ZM060496_at_Expressed | >gb\|ABF94602.1\|expressed protein [Oryza sativa |
| | | | protein | (japonica cultivar-group)] |
| 20 | 141 | Mnom000494 | −Zm.A1ZM012001_at_Putative | ref\|NP_001054859.1\|Os05g0194900 [Oryza |
| | | | 6-phospho- | sativa (japonica cultivar-group)] |
| | | | 1- | |
| | | | fructokinase | |
| 21 | 142 | Mnom000496 | −Zm.A1ZM061308_at_Putative | ref\|NP_001058018.1\|Os06g0604500 [Oryza |
| | | | small calcium | sativa (japonica cultivar-group)] |
| | | | binding | |
| | | | mitochondrial | |
| | | | carrier 2 | |
| 22 | 143 | Mnom000497 | −Zm.A1ZM073603_at_Putative | gb\|EAZ10866.1\|hypothetical protein OsJ_000691 |
| | | | 6-phospho- | [Oryza sativa (japonica cultivar-group)] |
| | | | 1- | |
| | | | fructokinase | |
| 23 | 144 | Mnom000499 | −Zm.A1ZM060016_at_Os02g0642600 | gb\|EAZ23967.1\|hypothetical protein OsJ_007450 |
| | | | protein | [Oryza sativa (japonica cultivar-group)] |
| 24 | 145 | Mnom000517 | +Gh.MRT363517117P.2 | gb\|AAY67798.1\|14-3-3 protein [Manihot |
| | | | | esculenta] |
| 25 | 146 | Mnom000554 | +Mt.CGPG2117 | unnamed protein product [Vitis vinifera] |
| 26 | 147 | Mnom000570 | +At.SIZ1 | ATSIZ1/SIZ1; DNA binding [Arabidopsis |
| | | | | thaliana] |
| 27 | 148 | Mnom000580 | +Gh.SP26 | alpha-crystalline heat shock protein [Gossypium |
| | | | | arboreum] gb\|ABI97959.1\|alpha-crystalline heat |
| | | | | shock protein [Gossypium arboreum] |
| 28 | 149 | Mnom000616-Mnom000617 | +At.XDH1 | ATXDH1 (XANTHINE DEHYDROGENASE |
| | | | | 1); xanthine dehydrogenase [Arabidopsis |
| | | | | thaliana] gb\|AAO11781.1\|xanthine |
| | | | | dehydrogenase 1 [Arabidopsis thaliana] |
| 29 | 150 | Mnom000618-Mnom000619 | +At.XDH2 | xanthine dehydrogenase 2 [Arabidopsis thaliana] |
| 30 | 151 | Mnom000626-Mnom000627 | +Zm.GA2ox | Os01g0209700 [Oryza sativa (japonica cultivar- |
| | | | | group)] dbj\|BAA96178.1\|putative GA 2-oxidase |
| | | | | 5 [Oryza 8sativa (japonica cultivar-group)] |
| | | | | dbj\|BAF04278.1\|Os01g0209700 [Oryza sativa |
| | | | | (japonica cultivar-group)] |
| 31 | 152 | Mnom000628 | −Zm.GA2ox | Os01g0209700 [Oryza sativa (japonica cultivar- |
| | | | | group)] dbj\|BAA96178.1\|putative GA 2-oxidase |
| | | | | 5 [Oryza sativa (japonica cultivar-group)] |
| | | | | dbj\|BAF04278.1\|Os01g0209700 [Oryza sativa |
| | | | | (japonica cultivar-group)] |
| 32 | 153 | Mnom000636 | +Zm.PHD finger | hypothetical protein OsJ_019708 [Oryza sativa |
| | | | protein-like | (japonica cultivar-group)] |
| 33 | 154 | Mnom000637 | +Zm.04g0442000 | Os04g0442000 [Oryza sativa (japonica cultivar- |
| | | | | group)] sp\|Q0JCZ4\|ARFI_ORYSJ Auxin |
| | | | | response factor 9 dbj\|BAF14793.1\| |
| | | | | Os04g0442000 [Oryza sativa (japonica cultivar- |
| | | | | group)] |
| 34 | 155 | Mnom000640 | +Zm.P0415C01.9 | unknown protein [Oryza sativa (japonica cultivar- |
| | | | | group)] |
| 35 | 156 | Mnom000641 | +Zm.5′-3′ | Os03g0794800 [Oryza sativa (japonica cultivar- |
| | | | exoribonuclease, | group)] gb\|AAT76426.1\|putative 5′-3′ |
| | | | putative, | exonuclease [Oryza sativa (japonica cultivar- |
| | | | expressed | group)] gb\|ABF99326.1\|5′-3′ exoribonuclease, |
| | | | | putative, expressed [Oryza sativa (japonica |
| | | | | cultivar-group)] dbj\|BAF13456.1\|Os03g0794800 |
| | | | | [Oryza sativa (japonica cultivar-group)] |
| 36 | 157 | Mnom000643 | +Zm.Putative | Os06g0149900 [Oryza sativa (japonica cultivar- |
| | | | cysteine synthase | group)] dbj\|BAD69043.1\|putative cysteine |
| | | | | synthase [Oryza sativa (japonica cultivar-group)] |
| | | | | dbj\|BAF18734.1\|Os06g0149900 [Oryza sativa |
| | | | | (japonica cultivar-group)] |
| 37 | 158 | Mnom000645 | +Zm.Putative | Os10g0475000 [Oryza sativa (japonica cultivar- |
| | | | oxidase | group)] gb\|AAL31049.1\|AC078893_12 putative |
| | | | | oxidase [Oryza sativa] gb\|ABB47786.2\|GMC |
| | | | | oxidoreductase family protein, expressed [Oryza |
| | | | | sativa (japonica cultivar-group)] dbj\|BAF26755.1\| |
| | | | | Os10g0475000 [Oryza sativa (japonica cultivar- |
| | | | | group)] |
| 38 | 159 | Mnom000647 | +Zm.Serine | Serine carboxypeptidase family protein, |
| | | | carboxypeptidase | expressed [Oryza sativa (japonica cultivar-group)] |
| | | | family protein, | |
| | | | expressed | |
| 39 | 160 | Mnom000682 | +At.SQD2 | SQD2 |
| | | | | (SULFOQUINOVOSYLDIACYLGLYCEROL |
| | | | | 2); UDP-sulfoquinovose:DAG |
| | | | | sulfoquinovosyltransferase/transferase, |
| | | | | transferring glycosyl groups [Arabidopsis |
| | | | | thaliana] gb\|AAM18913.1\|AF454354_1 sulfolipid |
| | | | | synthase [Arabidopsis thaliana] gb\|AAO64198.1\| |
| | | | | unknown protein [Arabidopsis thaliana] |
| 40 | 161 | Mnom000690 | +As.2-cys | 2Cys-peroxiredoxin precursor [Brassica rapa] |
| | | | peroxiredoxin | |
| | | | CTP fused to soy | |
| | | | 2-cys | |
| | | | peroxiredoxin | |
| | | | enzyme domain | |
| 41 | 162 | Mnom000107 | +Zm.phyA2 | phytochrome A2 [Zea mays] |
| 42 | 163 | Mnom000108 | +Zm.phyA2 | phytochrome A2 [Zea mays] |
| 43 | 164 | Mnom000109 | +Zm.phyA2 | phytochrome A2 [Zea mays] |
| 44 | 165 | Mnom000110 | +Zm.phyB1 | phytochrome B1 apoprotein; PhyB1 [Zea mays] |
| 45 | 166 | Mnom000111 | +Zm.PhyB1 | phytochrome B1 apoprotein; PhyB1 [Zea mays] |
| 46 | 167 | Mnom000112 | +Zm.PhyB1 | phytochrome B1 apoprotein; PhyB1 [Zea mays] |
| 47 | 168 | Mnom000113, | +Zm.PhyA2 | phytochrome A2 [Zea mays] |
| | | Mnom000116 | | |
| 48 | 169 | Mnom000114, | +Zm.PhyB1 | phytochrome B1 apoprotein; PhyB1 [Zea mays] |
| | | Mnom000117 | | |
| 49 | 170 | Mnom000115, | +Zm.PhyB1 | phytochrome B1 apoprotein; PhyB1 [Zea mays] |
| | | Mnom000118 | | |
| 50 | 171 | Mnom000119 | +Zm.phyA2 | phytochrome A2 [Zea mays] |
| 51 | 172 | Mnom000738 | +Zm.RTCL | RTCS-like protein [Zea mays] |
| 52 | 173 | Mnom000766-Mnom000767 | +Cr.MoT1 | molybdate transporter [Chlamydomonas |
| | | | | reinhardtii] |
| 53 | 174 | Mnom000822-Mnom000823 | −Zm.GW2 | RING-type E3 ubiquitin ligase [Oryza sativa |
| | | | | (indica cultivar-group)] |
| 54 | 175 | Mnom000857, | +Zm.wilt1256 | Os03g0291800 [Oryza sativa (japonica cultivar- |
| | | Mnom000858, | | group)] |
| | | Mnom000860 | | |
| 55 | 176 | Mnom000873 | +Th.CHIT33 | chitinase [Hypocrea lixii] |
| 56 | 177 | Mnom000886 | +Zm.SEC22 | putative vesicle trafficking protein [Oryza sativa |
| | | | | (japonica cultivar-group)] |
| 57 | 178 | Mnom000923-Mnom000924 | +Zm.phyB1 | phytochrome B1 apoprotein; PhyB1 [Zea mays] |
| 58 | 179 | Mnom000937 | +Zm.NPH1 | blue-light receptor phototropin 1 [Zea mays] |
| | | | | gb\|AAB88817.1\|nonphototropic hypocotyl 1 |
| | | | | [Zea mays] |
| 59 | 180 | Mnom000942, | +Os.TOP6A1 | putative topoisomerase 6 [Oryza sativa (japonica |
| | | Mnom000955-Mnom000956 | | cultivar-group)] |
| 60 | 181 | Mnom000985 | +Zm.A1ZM027162_at_OSJNBa0020I02.17 | hypothetical protein OsI_014890 [Oryza sativa |
| | | | protein | (indica cultivar-group)] |
| 61 | 182 | Mnom000986 | +Zm.A1ZM003077_s_at_Os10g0415800 | hypothetical protein OsI_032441 [Oryza sativa |
| | | | protein | (indica cultivar-group)] |
| 62 | 183 | Mnom001055 | +Zm.Serine | Serine carboxypeptidase family protein, |
| | | | carboxypeptidase | expressed [Oryza sativa (japonica cultivar-group)] |
| | | | family protein, | |
| | | | expressed | |
| 63 | 184 | Mnom001090-Mnom001092 | +At.Glutamine | GS2 (GLUTAMINE SYNTHETASE 2); |
| | | | synthetase | glutamate-ammonia ligase [Arabidopsis thaliana] |
| 64 | 185 | Mnom001138 | +Mt.UDPGPP | UDP-glucose pyrophosphorylase [Amorpha |
| | | | | fruticosa] |
| 65 | 186 | Mnom001140 | +Mt.DNAJ | unnamed protein product [Vitis vinifera] |
| | | | HEAT SHOCK | |
| | | | N-TERMINAL | |
| | | | DOMAIN- | |
| | | | CONTAINING | |
| | | | PROTEIN | |
| 66 | 187 | Mnom001141 | +Gh.DNAJ | DnaJ-like protein [Glycine max] |
| | | | HEAT SHOCK | |
| | | | N-TERMINAL | |
| | | | DOMAIN- | |
| | | | CONTAINING | |
| | | | PROTEIN | |
| 67 | 188 | Mnom001142 | −Gm.CGPG1284 | nicotinate phosphoribosyltransferase-like protein |
| | | | homologue | [Medicago truncatula] |
| 68 | 189 | Mnom001146 | +Zm.G1073-like 1 | Os02g0713700 [Oryza sativa (japonica cultivar- |
| | | | | group)] |
| 69 | 190 | Mnom001147 | −Zm.G1073-like 1 | Os02g0713700 [Oryza sativa (japonica cultivar- |
| | | | | group)] |
| 70 | 191 | Mnom001160 | +Zm.Znf1 | zinc finger protein [Zea mays] |
| 71 | 192 | Mnom001162 | +Zm.CSLD1 | putative cellulose synthase [Oryza sativa |
| | | | | (japonica cultivar-group)] |
| 72 | 193 | PHE0000597 | +Ec.mannitol-1- | mannitol-1-phosphate 5-dehydrogenase |
| | | | phosphate 5- | [Escherichia coli CFT073] |
| | | | dehydrogenase | gb\|AAN82853.1\|AE016768_271 Mannitol-1- |
| | | | | phosphate 5-dehydrogenase [Escherichia coli |
| | | | | CFT073] |
| 73 | 194 | PHE0001556 | +Np.cphA | COG0769: UDP-N-acetylmuramyl tripeptide |
| | | | | synthase [Nostoc punctiforme PCC 73102] |
| 74 | 195 | PHE0001557 | +Ss.cphA- | hypothetical protein slr2002 [Synechocystis sp. |
| | | | 1652956 | PCC 6803] |
| | | | | sp\|P73833\|CPHA_SYNY3 Cyanophycin |
| | | | | synthetase |
| | | | | dbj\|BAA17890.1\|slr2002 [Synechocystis sp. |
| | | | | PCC 6803] |
| 75 | 196 | PHE0001559 | +Ss.cphB- | hypothetical protein slr2001 [Synechocystis sp. |
| | | | 1652956 | PCC 6803] |
| | | | | sp\|P73832\|CPHB_SYNY3 Cyanophycinase |
| | | | | dbj\|BAA17889.1\|slr2001 [Synechocystis sp. |
| | | | | PCC 6803] |
| 76 | 197 | PHE0002862 | +Os.Senescence | Os05g0148700 [Oryza sativa (japonica cultivar- |
| | | | Associated | group)] gb\|AAS46022.1\|PSAG protein; PSAG1 |
| | | | Protein | [Oryza sativa (japonica cultivar-group)] |
| | | | | gb\|AAS46023.1\|PSAG protein; OsPSAG [Oryza |
| | | | | sativa (japonica cultivar-group)] gb\|AAU44024.1\| |
| | | | | unknown protein [Oryza sativa (japonica cultivar- |
| | | | | group)] dbj\|BAF16565.1\|Os05g0148700 [Oryza |
| | | | | sativa (japonica cultivar-group)] |
| 77 | 198 | PHE0003348 | +At.PIF4 | PIF4 (PHYTOCHROME INTERACTING |
| | | | | FACTOR 4); DNA binding/transcription factor |
| | | | | [Arabidopsis thaliana] sp\|Q8W2F3\|PIF4_ARATH |
| | | | | Phytochrome-interacting factor 4 (Basic helix- |
| | | | | loop-helix protein 9) (bHLH 9) (AtbHLH009) |
| | | | | (Short under red-light 2) |
| | | | | gb\|AAL55716.1\|AF251694_1 putative |
| | | | | transcription factor BHLH9 [Arabidopsis |
| | | | | thaliana] emb\|CAD29449.1\|phytochrome |
| | | | | interacting factor 4 [Arabidopsis thaliana] |
| 78 | 199 | PHE0003706 | +Sc.DUR3 | Dur3 |
| 79 | 200 | PHE0003708 | +Sp.DUR3 | hypothetical protein SPBC23G7.13c |
| | | | | [Schizosaccharomyces pombe 972h-] |
| | | | | emb\|CAA22629.1\|urea transporter (predicted) |
| | | | | [Schizosaccharomyces pombe] |
| 80 | 201 | PHE0004230 | +Zm.E2F3 like | Os02g0537500 [Oryza sativa (japonica cultivar- |
| | | | | group)] dbj\|BAD73815.1\|putative E2F homolog |
| | | | | [Oryza sativa (japonica cultivar-group)] |
| | | | | dbj\|BAF08963.1\|Os02g0537500 [Oryza sativa |
| | | | | (japonica cultivar-group)] |
| 81 | 202 | PHE0004820 | +Zm. | hypothetical protein OsJ_008481 [Oryza sativa |
| | | | MRT4577_21665C- | (japonica cultivar-group)] |
| | | | like ZmPIF3- | |
| | | | like family gene | |
| | | | in branch 1 | |
| 82 | 203 | PHE0004853 | −Zm.Wee1 | Wee1-like protein [Zea mays] |
| | | | fragment | |
| 83 | 204 | PHE0006017 | +Gh.Na/H | Na/H antiporter [Gossypium hirsutum] |
| | | | antiporter | |
| 84 | 205 | PHE0006102 | +Sc.CGPG8909 | Constituent of 66S pre-ribosomal particles, |
| | | | SSF1_YEAST | required for ribosomal |
| | | | Ribosome | large subunit maturation; functionally redundant |
| | | | biogenesis | with |
| | | | protein | Ssf2p; member of the Brix family |
| | | | | [Saccharomyces |
| | | | | cerevisiae] |
| 85 | 206 | PHE0006378 | +Sc.Rrp40p | YOL142W [Saccharomyces cerevisiae] |
| 86 | 207 | PHE0006380 | +Sc.Rrp46p | Protein involved in rRNA processing; component |
| | | | | of the exosome 3->5 exonuclease complex; |
| | | | | Rrp46p [Saccharomyces cerevisiae] |
| | | | | sp\|P53256\|RRP46_YEAST Exosome complex |
| | | | | exonuclease RRP46 (Ribosomal RNA-processing |
| | | | | protein 46) gb\|EDN61685.1\|3′-5′-exoribonuclease |
| | | | | [Saccharomyces cerevisiae YJM789] |
| 87 | 208 | PHE0006496 | −Zm.KNAT6 | N/A |
| | | | New RNAi | |
| 88 | 209 | PHE0006610 | +Gm.Glutathione | Phospholipid hydroperoxide glutathione |
| | | | Peroxidase | peroxidase, chloroplast precursor (PHGPx) |
| | | | | emb\|CAA04142.1\|phospholipid glutathione |
| | | | | peroxidase [Pisum sativum] |
| 89 | 210 | PHE0006613 | +Gm. | Os03g0358100 [Oryza sativa (japonica cultivar- |
| | | | Glutathione | group)] emb\|CAC17628.1\|putative phospholipid |
| | | | Peroxidase | hydroperoxide glutathione peroxidase [Oryza |
| | | | | sativa (japonica cultivar-group)] gb\|ABF96054.1\| |
| | | | | glutathione peroxidase, putative, expressed |
| | | | | [Oryza sativa (japonica cultivar-group)] |
| | | | | dbj\|BAF12059.1\|Os03g0358100 [Oryza sativa |
| | | | | (japonica cultivar-group)] gb\|EAY90064.1\| |
| | | | | hypothetical protein OsI_011297 [Oryza sativa |
| | | | | (indica cultivar-group)] gb\|EAZ26982.1\| |
| | | | | hypothetical protein OsJ_010465 [Oryza sativa |
| | | | | (japonica cultivar-group)] |
| 90 | 211 | PHE0006916 | +Zm.A1ZM059972_at_Ribosome | N/A |
| | | | associated | |
| | | | membrane | |
| | | | protein RAMP4 | |
| 91 | 212 | PHE0006917 | +Zm.A1ZM057844_at_26S | Os06g0633100 [Oryza sativa (japonica cultivar- |
| | | | protease | group)] |
| | | | regulatory | dbj\|BAD37803.1\|unknown protein [Oryza sativa |
| | | | subunit 7 (26S | (japonica cultivar-group)] |
| | | | proteasome | dbj\|BAF20043.1\|Os06g0633100 [Oryza sativa |
| | | | subunit 7) | (japonica cultivar-group)] |
| 92 | 213 | PHE0007643 | +At.CGPG7785_At4g22730 | leucine-rich repeat transmembrane protein kinase, |
| | | | emb\|CAB79228.1\| | putative [Arabidopsis thaliana] emb\|CAA16558.1\| |
| | | | leucine rich | leucine rich repeat receptor kinase-like protein |
| | | | repeat | [Arabidopsis thaliana] emb\|CAB79228.1\|leucine |
| | | | | rich repeat receptor kinase-like protein |
| | | | | [Arabidopsis thaliana] gb\|AAR23717.1\| |
| | | | | At4g22730 [Arabidopsis thaliana] |
| | | | | dbj\|BAD44629.1\|leucine rich repeat receptor |
| | | | | kinase-like protein [Arabidopsis thaliana] |
| 93 | 214 | PHE0008287 | +Zm.CGPG7793 | Os05g0154900 [Oryza sativa (japonica cultivar- |
| | | | | group)] gb\|AAV32216.1\|unknown protein [Oryza |
| | | | | sativa (japonica cultivar-group)] dbj\|BAF16600.1\| |
| | | | | Os05g0154900 [Oryza sativa (japonica cultivar- |
| | | | | group)] |
| 94 | 215 | PHE0008407 | +Ps.CGPG6515_succinate- | succinate-semialdehyde dehydrogenase |
| | | | semialdehyde | [Pseudomonas syringae pv. tomato str. DC3000] |
| | | | dehydrogenase | gb\|AAO53845.1\|succinate-semialdehyde |
| | | | | dehydrogenase [Pseudomonas syringae pv. |
| | | | | tomato str. DC3000] |
| 95 | 216 | PHE0008411 | +Zm.CGPG9112_hypothetical | hypothetical protein OsJ_012412 [Oryza sativa |
| | | | protein | (japonica cultivar-group)] |
| 96 | 217 | PHE0008541 | +Zm.Ctp-Hb1 | 3-phosphoshikimate 1-carboxyvinyltransferase/ |
| | | | | 5-enolpyruvylshikimate-3-phosphate/EPSP |
| | | | | synthase [Arabidopsis thaliana] gb\|AAB82633.1\| |
| | | | | 5-enolpyruvylshikimate-3-phosphate (EPSP) |
| | | | | synthase [Arabidopsis thaliana] gb\|AAY25438.1\| |
| | | | | At2g45300 [Arabidopsis thaliana] |
| | | | | dbj\|BAE99170.1\|5-enolpyruvylshikimate-3- |
| | | | | phosphate (EPSP) synthase [Arabidopsis thaliana] |
| 97 | 218 | PHE0008602 | +Xn.CGPG7423_glutamine | glutamine synthetase [Photorhabdus luminescens |
| | | | synthetase | subsp. laumondii TTO1] emb\|CAE12532.1\| |
| | | | (glutamate-- | glutamine synthetase (glutamate--ammonia |
| | | | ammonia ligase) | ligase) [Photorhabdus luminescens subsp. |
| | | | | laumondii TTO1] |
| 98 | 219 | PHE0009339 | +At.Luminal | BIP (LUMINAL BINDING PROTEIN); ATP |
| | | | binding protein 2 | binding [Arabidopsis thaliana] |
| | | | precursor | sp\|Q39043\|BIP2_ARATH Luminal-binding |
| | | | | protein 2 precursor (BiP2) (AtBP2) |
| | | | | dbj\|BAB08435.1\|luminal binding protein |
| | | | | [Arabidopsis thaliana] |
| | | | | gb\|AAO00752.1\|luminal binding protein |
| | | | | [Arabidopsis thaliana] |
| | | | | gb\|AAP37765.1\|At5g42020 [Arabidopsis |
| | | | | thaliana] |
| 99 | 220 | PHE0009341 | +Os.Putative | Os01g0307500 [Oryza sativa (japonica cultivar- |
| | | | high affinity | group)] sp\|Q3YAF4\|HKT8_ORYSA Cation |
| | | | potassium | transporter HKT8 (OsHKT8) |
| | | | transporter 2 | dbj\|BAB93392.1\|putative high affinity |
| | | | | potassium transporter 2 [Oryza sativa |
| | | | | (japonica cultivar-group)] |
| | | | | dbj\|BAD88380.1\|putative high affinity |
| | | | | potassium transporter 2 [Oryza sativa |
| | | | | (japonica cultivar-group)] |
| | | | | dbj\|BAF04762.1\|Os01g0307500 [Oryza sativa |
| | | | | (japonica cultivar-group)] |
| 100 | 221 | PHE0010195 | +At.Arabidopsis | oligopeptide transport (POT) family protein |
| | | | Peptide | [Arabidopsis thaliana] |
| | | | transporter | emb\|CAB87717.1\|putative oligopeptide |
| | | | | transporter protein [Arabidopsis thaliana] |
| 101 | 222 | PHE0010352 | +Gh.GhG481 | transcription factor NF-Y CCAAT-binding-like |
| | | | | protein-like [Solanum tuberosum] |
| | | | | gb\|ABB55391.1\|transcription factor NF-Y |
| | | | | CCAAT-binding-like protein-like [Solanum |
| | | | | tuberosum] gb\|ABB72810.1\|transcription factor |
| | | | | NF-Y, CCAAT-binding-like protein [Solanum |
| | | | | tuberosum] |
| 102 | 223 | PHE0010354 | +Gh.NFB2-1:1:1 | transcription factor NF-Y CCAAT-binding-like |
| | | | | protein-like [Solanum tuberosum] |
| | | | | gb\|ABB55391.1\|transcription factor NF-Y |
| | | | | CCAAT-binding-like protein-like [Solanum |
| | | | | tuberosum] gb\|ABB72810.1\|transcription factor |
| | | | | NF-Y, CCAAT-binding-like protein [Solanum |
| | | | | tuberosum] |
| 103 | 224 | PHE0010646 | +U.GOI- | spermidine synthase [Cucumis sativus] |
| | | | CUCfi.FSPD1:1:1 | |
| 104 | 225 | PHE0010648 | +Gh.G2041_Truncated | HR38 (chromatin remodeling 38); ATP binding/ |
| | | | | DNA binding/helicase [Arabidopsis |
| | | | | thaliana] emb\|CAB86450.1\|putative protein |
| | | | | [Arabidopsis thaliana |
| 105 | 226 | PHE0010649 | +Gm.G2041_Truncated | SNF2 domain-containing protein |
| | | | | [Olimarabidopsis pumila] |
| 106 | 227 | PHE0010852 | +Zm.Helix helix | hypothetical protein OsI_028787 [Oryza sativa |
| | | | loop | (indica cultivar-group)] |
| | | | protein_NC0035132: | |
| | | | YLD | |
| 107 | 228 | PHE0010956 | +At.ENT1 | ENT1, AT (EQUILIBRATIVE NUCLEOTIDE |
| | | | | TRANSPORTER 1); nucleoside |
| | | | | transporter [Arabidopsis thaliana] |
| | | | | gb\|AAL67041.1\|unknown protein [Arabidopsis |
| | | | | thaliana] |
| | | | | gb\|AAO22816.1\|unknown protein [Arabidopsis |
| | | | | thaliana] |
| 108 | 229 | PHE0011028 | +U.GOI- | N/A |
| | | | Nue1:1:1 | |
| 109 | 230 | PHE0011063 | +U.GOI-Nue1- | Non-symbiotic hemoglobin (Hbt) (ZEAmp |
| | | | Zm.Hb1RNA:1:1 | GLB1) gb\|AAF44664.1\|AF236080_1 hemoglobin |
| | | | | [Zea mays] gb\|AAG01183.1\|hemoglobin [Zea |
| | | | | mays subsp. parviglumis] |
| 110 | 231 | PHE0011543 | +Le.Tomato | zinc finger (B-box type) family protein |
| | | | Homolog of | [Arabidopsis thaliana] |
| | | | G1988 | |
| 111 | 232 | PHE0013202 | +At.basic helix- | PIL2 (PHYTOCHROME INTERACTING |
| | | | loop-helix | FACTOR 3-LIKE 2); transcription |
| | | | (bHLH) protein | factor [Arabidopsis thaliana] |
| | | | similar to PIF3- | |
| | | | like bHLH | |
| | | | protein 2 (PIL2) | |
| 112 | 233 | PHE0013203 | +At.sterile alpha | sterile alpha motif (SAM) domain-containing |
| | | | motif (SAM) | protein [Arabidopsis thaliana] |
| | | | domain- | |
| | | | containing | |
| | | | protein | |
| 113 | 234 | PHE0013205 | +At.DUF | unknown protein [Arabidopsis thaliana] |
| 114 | 235 | PHE0013292 | +At.ctpA | C-terminal processing protease, putative |
| | | | | [Arabidopsis thaliana] |
| 115 | 236 | PHE0013712 | +Zm.PROPEP2 | Os04g0638700 [Oryza sativa (japonica cultivar- |
| | | | | group)] |
| 116 | 237 | PHE0013713 | +Zm.PROPEP3 | Os04g0638700 [Oryza sativa (japonica cultivar- |
| | | | | group)] |
| 117 | 238 | PHE0013734 | +At.AGL94 | DNA binding/transcription factor [Arabidopsis |
| | | | | thaliana] |
| 118 | 239 | TRG0000049 | −Zm.NFB5 | hypothetical protein OsI_019577 [Oryza sativa |
| | | | | (indica cultivar-group)] |
| 119 | 240 | TRG0000291 | −Zm.coumaryl | AMP-binding enzyme family protein, expressed |
| | | | ligase | [Oryza sativa (japonica cultivar-group)] |
| 120 | 241 | TRG0000332 | −Gm.NFA9.3.1- | unnamed protein product [Vitis vinifera] |
| | | | Soy homolog of | |
| | | | AtG926 | |
| 121 | 242 | 1071693 | +Zm.PhyB | phytochrome B2 apoprotein; PhyB2 [Zea mays] |

Selection Methods for Transgenic Plants with Enhanced Agronomic Trait

Within a population of transgenic plants each regenerated from a plant cell having a nucleus with recombinant DNA many plants that survive to fertile transgenic plants that produce seeds and progeny plants will not exhibit an enhanced agronomic trait. Selection from the population is necessary to identify one or more transgenic plant cells having a transgenic nucleus that can provide plants with the enhanced trait. Transgenic plants having enhanced traits are selected from populations of plants regenerated or derived from plant cells transformed as described herein by evaluating the plants in a variety of assays to detect an enhanced trait, e.g. enhanced water use efficiency, enhanced cold tolerance, increased yield, enhanced nitrogen use efficiency, enhanced seed protein and enhanced seed oil. These assays also may take many forms including, but not limited to, direct screening for the trait in a greenhouse or field trial or by screening for a surrogate trait. Such analyses can be directed to detecting changes in the chemical composition, biomass, physiological properties, morphology of the plant. Changes in chemical compositions such as nutritional composition of grain can be detected by analysis of the seed composition and content of protein, free amino acids, oil, free fatty acids, starch or tocopherols. Changes in biomass characteristics can be made on greenhouse or field grown plants and can include plant height, stem diameter, root and shoot dry weights; and, for corn plants, ear length and diameter. Changes in physiological properties can be identified by evaluating responses to stress conditions, for example assays using imposed stress conditions such as water deficit, nitrogen deficiency, cold growing conditions, pathogen or insect attack or light deficiency, or increased plant density. Changes in morphology can be measured by visual observation of tendency of a transformed plant with an enhanced agronomic trait to also appear to be a normal plant as compared to changes toward bushy, taller, thicker, narrower leaves, striped leaves, knotted trait, chlorosis, albino, anthocyanin production, or altered tassels, ears or roots. Other selection properties include days to pollen shed, days to silking, leaf extension rate, chlorophyll content, leaf temperature, stand, seedling vigor, internode length, plant height, leaf number, leaf area, tillering, brace roots, stay green, stalk lodging, root lodging, plant health, barreness/prolificacy, green snap, and pest resistance. In addition, phenotypic characteristics of harvested grain may be evaluated, including number of kernels per row on the ear, number of rows of kernels on the ear, kernel abortion, kernel weight, kernel size, kernel density and physical grain quality.

Assays for screening for a desired trait are readily designed by those practicing in the art. The following illustrates useful screening assays for corn traits using hybrid corn plants. The assays can be readily adapted for screening other plants such as canola, cotton and soybean either as hybrids or inbreds.

Transgenic corn plants having nitrogen use efficiency are identified by screening in fields with three levels of nitrogen (N) fertilizer being applied, e.g. low level (0 N), medium level (80 lb/ac) and high level (180 lb/ac). Plants with enhanced nitrogen use efficiency provide higher yield as compared to control plants.

Transgenic corn plants having enhanced yield are identified by screening using progeny of the transgenic plants over multiple locations with plants grown under optimal production management practices and maximum weed and pest control. A useful target for improved yield is a 5% to 10% increase in yield as compared to yield produced by plants grown from seed for a control plant. Selection methods may be applied in multiple and diverse geographic locations, for example up to 16 or more locations, over one or more planting seasons, for example at least two planting seasons, to statistically distinguish yield improvement from natural environmental effects.

Transgenic corn plants having enhanced water use efficiency are identified by screening plants in an assay where water is withheld for a period to induce stress followed by watering to revive the plants. For example, a useful selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment.

Transgenic corn plants having enhanced cold tolerance are identified by screening plants in a cold germination assay and/or a cold tolerance field trial. In a cold germination assay trays of transgenic and control seeds are placed in a growth chamber at 9.7° C. for 24 days (no light). Seeds having higher germination rates as compared to the control are identified as having enhanced cold tolerance. In a cold tolerance field trial plants with enhanced cold tolerance are identified from field planting at an earlier date than conventional Spring planting for the field location. For example, seeds are planted into the ground around two weeks before local farmers begin to plant corn so that a significant cold stress is exerted onto the crop, named as cold treatment. Seeds also are planted under local optimal planting conditions such that the crop has little or no exposure to cold condition, named as normal treatment. At each location, seeds are planted under both cold and normal conditions preferably with multiple repetitions per treatment.

Transgenic corn plants having seeds with increased protein and/or oil levels are identified by analyzing progeny seed for protein and/or oil. Near-infrared transmittance spectrometry is a non-destructive, high-throughput method that is useful to determine the composition of a bulk seed sample for properties listed in table 2.

TABLE 2

| | |
|---|---|
| Typical sample(s): | Whole grain corn and soybean seeds |
| Typical analytical range: | Corn - moisture 5-15%, oil 5-20%, |
| | protein 5-30%, starch 50-75%, and density |
| | 1.0-1.3%. |
| | Soybean - moisture 5-15%, oil 15-25%, |
| | and protein 35-50%. |

Although the plant cells and methods of this invention can be applied to any plant cell, plant, seed or pollen, e.g. any fruit, vegetable, grass, tree or ornamental plant, the various aspects of the invention are preferably applied to corn, soybean, cotton, canola, alfalfa, wheat, rice, sugarcane, and sugar beet plants. In many cases the invention is applied to corn plants that are inherently resistant to disease from the Mal de Rio Cuarto virus or the Puccina sorghi fungus or both.

The following examples are included to demonstrate aspects of the invention, those of skill in the art should, in light of the present disclosure, appreciate that many changes can be made in the specific aspects which are disclosed and still obtain a like or similar results without departing from the spirit and scope of the invention.

### Example 1

### Plant Expression Constructs

This example illustrates the construction of plasmids for transferring recombinant DNA into a plant cell nucleus that can be regenerated into transgenic plants.

### A. Plant Expression Constructs for Corn Transformation

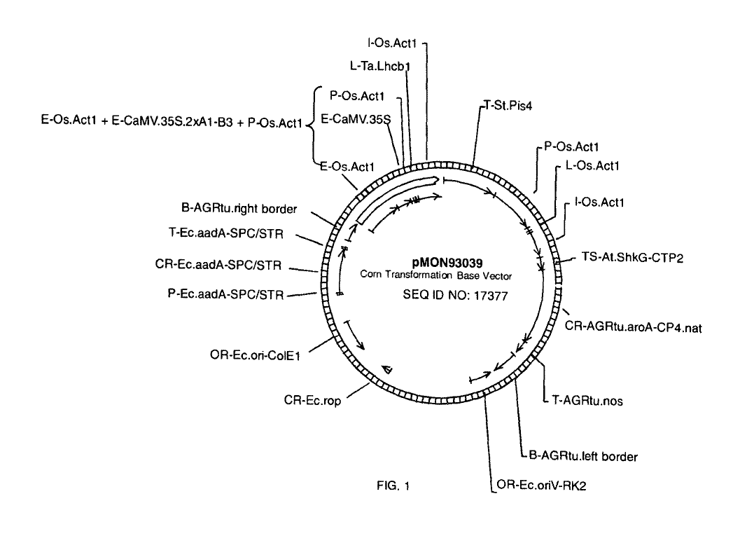

A base corn transformation vector pMON93039, as set forth in SEQ ID NO:17377, illustrated in Table 3 and FIG. 1, is fabricated for use in preparing recombinant DNA for Agrobacterium-mediated transformation into corn tissue.

TABLE 3

| | | | Coordinates of |
|---|---|---|---|
| Function | Name | Annotation | SEQ ID NO: 17377 |
| Agrobacterium | B-AGRtu.right border | Agro right border sequence, | 11364-11720 |
| T-DNA transfer | | essential for transfer of T-DNA. | |
| Gene of interest | E-Os.Act1 | Upstream promoter region of the | 19-775 |
| expression | | rice actin 1 gene | |
| cassette | E-CaMV.35S.2xA1- | Duplicated35S A1-B3 domain | 788-1120 |
| | B3 | without TATA box | |
| | P-Os.Act1 | Promoter region of the rice actin 1 | 1125-1204 |
| | | gene | |
| | L-Ta.Lhcb1 | 5′ untranslated leader of wheat | 1210-1270 |
| | | major chlorophyll a/b binding | |
| | | protein | |
| | I-Os.Act1 | First intron and flanking UTR exon | 1287-1766 |
| | | sequences from the rice actin 1 | |
| | | gene | |
| | T-St.Pis4 | 3′ non-translated region of the | 1838-2780 |
| | | potato proteinase inhibitor II gene | |
| | | which functions to direct | |
| | | polyadenylation of the mRNA | |
| Plant selectable | P-Os.Act1 | Promoter from the rice actin 1 gene | 2830-3670 |
| marker | L-Os.Act1 | First exon of the rice actin 1 gene | 3671-3750 |
| expression | I-Os.Act1 | First intron and flanking UTR exon | 3751-4228 |
| cassette | | sequences from the rice actin 1 | |
| | | gene | |
| | TS-At.ShkG-CTP2 | Transit peptide region of | 4238-4465 |
| | | Arabidopsis EPSPS | |
| | CR-AGRtu.aroA- | Coding region for bacterial strain | 4466-5833 |
| | CP4.nat | CP4 native aroA gene. | |
| | T-AGRtu.nos | A 3′ non-translated region of the | 5849-6101 |
| | | nopaline synthase gene of | |
| | | Agrobacterium tumefaciens Ti | |
| | | plasmid which functions to direct | |
| | | polyadenylation of the mRNA. | |
| Agrobacterium | B-AGRtu.left border | Agro left border sequence, essential | 6168-6609 |
| T-DNA transfer | | for transfer of T-DNA. | |
| Maintenance in | OR-Ec.oriV-RK2 | The vegetative origin of replication | 6696-7092 |
| E. coli | | from plasmid RK2. | |
| | CR-Ec.rop | Coding region for repressor of | 8601-8792 |
| | | primer from the ColE1 plasmid. | |
| | | Expression of this gene product | |
| | | interferes with primer binding at the | |
| | | origin of replication, keeping | |
| | | plasmid copy number low. | |
| | OR-Ec.ori-ColE1 | The minimal origin of replication | 9220-9808 |
| | | from the E. coli plasmid ColE1. | |
| | P-Ec.aadA-SPC/STR | Promoter for Tn7 | 10339-10380 |
| | | adenylyltransferase (AAD(3″)) | |
| | CR-Ec.aadA- | Coding region for Tn7 | 10381-11169 |
| | SPC/STR | adenylyltransferase (AAD(3″)) | |
| | | conferring spectinomycin and | |
| | | streptomycin resistance. | |
| | T-Ec.aadA-SPC/STR | 3′ UTR from the Tn7 | 11170-11227 |
| | | adenylyltransferase (AAD(3″)) | |
| | | gene of E. coli. | |

To construct transformation vectors for expressing a protein identified in Table 1, primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the intron element (coordinates 1287-1766) and the polyadenylation element (coordinates 1838-2780).

To construct transformation vectors for suppressing a protein identified in Table 1, the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. More specifically, the sense and anti-sense DNA is derived from an endogenous corn gene that expresses a corn protein with an amino acid sequence of SEQ ID NO: 141-144, 152, 174, 190, 203, 208, or 239-240 or the corn homolog of SEQ ID NOs:122, 188, or 241.

### B. Plant Expression Constructs for Soy and Canola Transformation

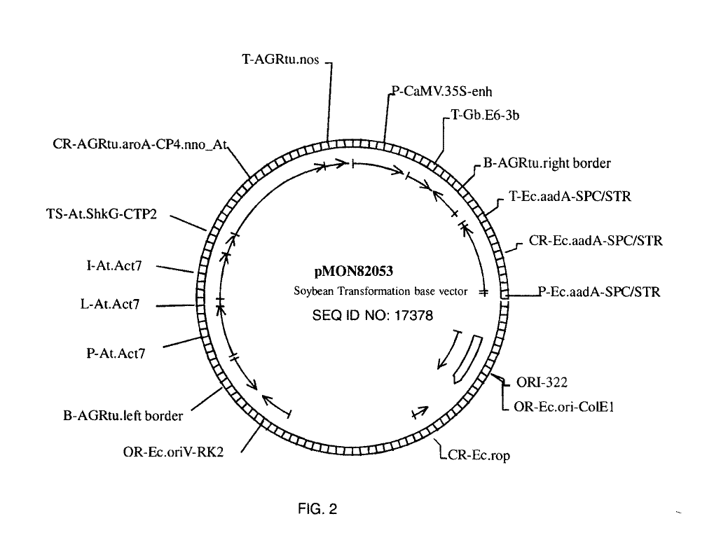

Vectors for use in transformation of soybean and canola tissue are prepared having the elements of expression vector pMON82053 (SEQ ID NO: 17378) as shown in Table 4 below and FIG. 2.

TABLE 4

| | | | Coordinates of |
|---|---|---|---|
| Function | Name | Annotation | SEQ ID NO: 17378 |
| Agrobacterium T- | B-AGRtu.left border | Agro left border sequence, essential for | 6144-6585 |
| DNA transfer | | transfer of T-DNA. | |
| Plant selectable | P-At.Act7 | Promoter from the Arabidopsis actin 7 gene | 6624-7861 |
| marker expression | L-At.Act7 | 5′UTR of Arabidopsis Act7 gene | |
| cassette | I-At.Act7 | Intron from the Arabidopsis actin7 gene | |
| | TS-At.ShkG-CTP2 | Transit peptide region of Arabidopsis | 7864-8091 |
| | | EPSPS | |
| | CR-AGRtu.aroA- | Synthetic CP4 coding region with dicot | 8092-9459 |
| | CP4.nno_At | preferred codon usage. | |
| | T-AGRtu.nos | A 3′ non-translated region of the nopaline | 9466-9718 |
| | | synthase gene of Agrobacterium | |
| | | tumefaciens Ti plasmid which functions to | |
| | | direct polyadenylation of the mRNA. | |
| Gene of interest | P-CaMV.35S-enh | Promoter for 35S RNA from CaMV | 1-613 |
| expression cassette | | containing a duplication of the −90 to −350 | |
| | | region. | |
| | T-Gb.E6-3b | 3′ untranslated region from the fiber protein | 688-1002 |
| | | E6 gene of sea-island cotton. | |
| Agrobacterium T- | B-AGRtu.right | Agro right border sequence, essential for | 1033-1389 |
| DNA transfer | border | transfer of T-DNA. | |
| Maintenance in E. coli | OR-Ec.oriV-RK2 | The vegetative origin of replication from | 5661-6057 |
| | | plasmid RK2. | |
| | CR-Ec.rop | Coding region for repressor of primer from | 3961-4152 |
| | | the ColE1 plasmid. Expression of this gene | |
| | | product interferes with primer binding at the | |
| | | origin of replication, keeping plasmid copy | |
| | | number low. | |
| | OR-Ec.ori-ColE1 | The minimal origin of replication from the | 2945-3533 |
| | | E. coli plasmid ColE1. | |
| | P-Ec.aadA-SPC/STR | Promoter for Tn7 adenylyltransferase | 2373-2414 |
| | | (AAD(3″)) | |
| | CR-Ec.aadA- | Coding region for Tn7 adenylyltransferase | 1584-2372 |
| | SPC/STR | (AAD(3″)) conferring spectinomycin and | |
| | | streptomycin resistance. | |
| | T-Ec.aadA-SPC/STR | 3′ UTR from the Tn7 adenylyltransferase | 1526-1583 |
| | | (AAD(3″)) gene of E. coli. | |

To construct transformation vectors for expressing a protein identified in Table 1, primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the promoter element (coordinates 1-613) and the polyadenylation element (coordinates 688-1002).

To construct transformation vectors for suppressing a protein identified in Table 1, the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. More specifically, for soybean the sense and anti-sense DNA is derived from an endogenous soybean gene that expresses a soybean protein with an amino acid sequence of SEQ ID NOs: 122, 188, or 241 or the soybean homolog of SEQ ID NOs: 141-144, 152, 174, 190, 203, 208, or 239-240, and for canola the sense and anti-sense DNA is derived from an endogenous canola gene that encodes the canola homolog of SEQ ID NOs: 122, 141-144, 152, 174, 188, 190, 203, 208, or 239-241.

### C. Cotton Transformation Vector

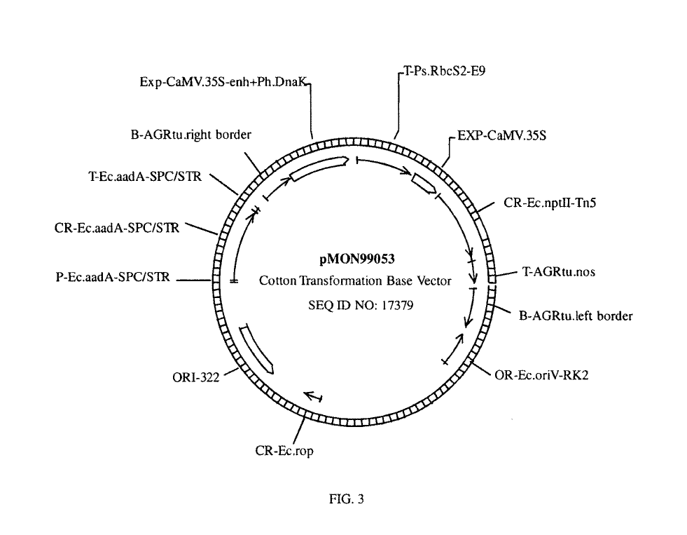

Plasmids for use in transformation of cotton tissue are prepared with elements of expression vector pMON99053 (SEQ ID NO: 17379) as shown in Table 5 below and FIG. 3.

TABLE 5

| | | | Coordinates of |
|---|---|---|---|
| | | | SEQ ID NO: |
| Function | Name | Annotation | 17379 |
| Agrobacterium T- | B-AGRtu.right border | Agro right border sequence, essential for | 1-357 |
| DNA transfer | | transfer of T-DNA. | |
| Gene of interest | Exp-CaMV.35S- | Enhanced version of the 35S RNA promoter | 388-1091 |
| expression cassette | enh + Ph.DnaK | from CaMV plus the petunia hsp70 5′ | |
| | | untranslated region | |
| | T-Ps.RbcS2-E9 | The 3′ non-translated region of the pea RbcS2 | 1165-1797 |
| | | gene which functions to direct | |
| | | polyadenylation of the mRNA. | |
| Plant selectable | Exp-CaMV.35S | Promoter and 5′ untranslated region from the | 1828-2151 |
| marker expression | | 35S RNA of CaMV | |
| cassette | CR-Ec.nptII-Tn5 | Coding region for neomycin | 2185-2979 |
| | | phosphotransferase gene from transposon Tn5 | |
| | | which confers resistance to neomycin and | |
| | | kanamycin. | |
| | T-AGRtu.nos | A 3′ non-translated region of the nopaline | 3011-3263 |
| | | synthase gene of Agrobacterium tumefaciens | |
| | | Ti plasmid which functions to direct | |
| | | polyadenylation of the mRNA. | |
| Agrobacterium T- | B-AGRtu.left border | Agro left border sequence, essential for | 3309-3750 |
| DNA transfer | | transfer of T-DNA. | |
| Maintenance in E. coli | OR-Ec.oriV-RK2 | The vegetative origin of replication from | 3837-4233 |
| | | plasmid RK2. | |
| | CR-Ec.rop | Coding region for repressor of primer from | 5742-5933 |
| | | the ColE1 plasmid. Expression of this gene | |
| | | product interferes with primer binding at the | |
| | | origin of replication, keeping plasmid copy | |
| | | number low. | |
| | OR-Ec.ori-ColE1 | The minimal origin of replication from the E. coli | 6361-6949 |
| | | plasmid ColE1. | |
| | P-Ec.aadA-SPC/STR | Promoter for Tn7 adenylyltransferase | 7480-7521 |
| | | (AAD(3″)) | |
| | CR-Ec.aadA-SPC/STR | Coding region for Tn7 adenylyltransferase | 7522-8310 |
| | | (AAD(3″)) conferring spectinomycin and | |
| | | streptomycin resistance. | |
| | T-Ec.aadA-SPC/STR | 3′ UTR from the Tn7 adenylyltransferase | 8311-8368 |
| | | (AAD(3″)) gene of E. coli. | |

To construct transformation vectors for expressing a protein identified in Table 1, primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the promoter element (coordinates 388-1091) and the polyadenylation element (coordinates 1165-1797).

To construct transformation vectors for suppressing a protein identified in Table 1, the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. More specifically, the sense and anti-sense DNA is derived from an endogenous cotton gene that encodes the cotton homolog of SEQ ID NO: 122, 141-144, 152, 174, 188, 190, 203, 208, or 239-241.

### D. Plant Expression Constructs for Gene Stacking in Corn.

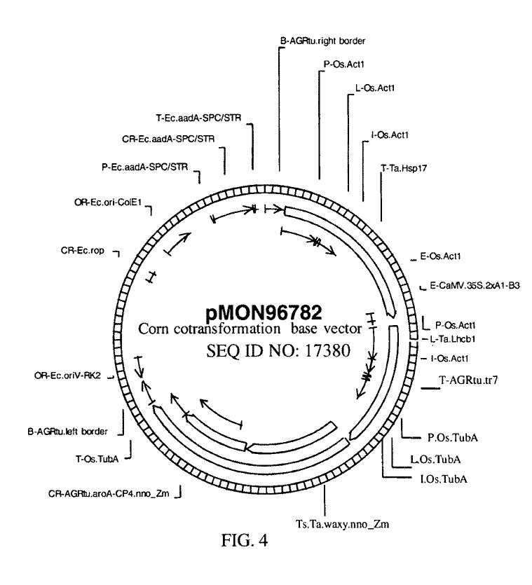

A base corn transformation vector pMON96782, as set forth in SEQ ID NO: 17380, illustrated in Table 6 and FIG. 4, is fabricated for use in preparing recombinant DNA for Agrobacterium-mediated transformation into corn tissue.

TABLE 6

| | | | Coordinates of |
|---|---|---|---|
| Function | Name | Annotation | SEQ ID NO: 17380 |
| Agrobacterium | B-AGRtu.right border | Agro right border sequence, | 1-357 |
| T-DNA transfer | | essential for transfer of T-DNA. | |
| Gene of interest | P-Os.Act1 | Promoter region of the rice actin 1 | 403-1243 |
| expression | | gene | |
| cassette 1 | L-Os.Act1 | 5′ untranslated leader of rice actin 1 | 1244-1323 |
| | | gene | |
| | I-Os.Act1 | First intron and flanking UTR exon | 1324-1801 |
| | | sequences from the rice actin 1 | |
| | | gene | |
| | T-Ta.Hsp17 | 3′ un-translated region of wheat low | 1834-2043 |
| | | molecular weight heat shock | |
| | | protein gene | |
| Gene of interest | E-Os.Act1 | Upstream Promoter region of rice | 2136-2892 |
| expression | | actin 1 | |
| cassette 2 | E-CaMV.35S.2xA1- | 35S A1-B3 Domain | 2905-2937 |
| | B3 | | |
| | P-Os.Act1 | Promoter from rice actin gene | 3242-3321 |
| | L-Ta.Lhcb1 | 5′ untranslated leader from wheat | 3327-3387 |
| | | chlorophyll protein | |
| | I-Os.Act1 | Intron and 5′ untranslated region | 3404-3883 |
| | | from rice actin 1 gene | |
| | T-AGRtu.tr7 | 3′ untranslated region from | 3918-4425 |
| | | “transcript 7” of Agrobacterium | |
| Plant selectable | P.Os.TubA | Promoter of alpha-tubulin gene of | 4452-5650 |
| marker | | rice | |
| expression | L.Os.TubA | 5′ untranslated region of an alpha | 5651-5736 |
| cassette | | tubulin from rice | |
| | I.Os.TubA | Intron 1 of an alpha tubulin from | 5737-6632 |
| | | rice. | |
| | Ts.Ta.waxy.nno_Zm | Chloroplast transit peptide from | 6637-6846 |
| | | wheat starch synthase | |
| | Cr.AGRtu.aroA- | CP4 EPSPS gene | 6847-8214 |
| | CP4.nno_Zm | | |
| | T.Os.TubA | 3′ untranslated region of alpha | 8219-8800 |
| | | tubulin from rice | |
| Agrobacterium | B-AGRtu.left border | Left border sequence for T-DNA | 8828-9269 |
| T-DNA transfer | | transfer | |
| Maintenance in | OR-Ec.oriV-RK2 | Origin of replication from the E. coli | 9356-9752 |
| E. coli | | plasmid RK2. | |
| | Cr-Ec.rop | Coding region for repressor of | 11261-11452 |
| | | primer from ColE1 plasmid | |
| | OR-Ec.ori-ColE1 | Minimum origin of replication from | 11880-12468 |
| | | E. coli colE1 plasmid. | |
| | P-Ec.aadA-SPC/STR | Promoter for Tn7 | 12999-13040 |
| | | adenylyltransferase gene | |
| | CR-Ec.aadA- | Coding region for Tn7 | 13041-13829 |
| | SPC/STR | adenylyltransferase gene | |
| | T-Ec.aadA-SPC/STR | 3′ untranslated region from Tn7 | 13830-13887 |
| | | adenylyltransferase gene | |

Primers for PCR amplification of protein coding nucleotides of the genes of interest are designed at or near the start and stop codons of the coding sequence, in order to eliminate most of the 5′ and 3′ untranslated regions. Protein coding regions of genes encoding a first and second protein of interest are amplified. The amplified region from the first gene of interest is cloned between nucleotides 1801 and 1834 of the base vector and the amplified region from the second gene of interest is cloned between nucleotides 3883 and 3918 of the base vector.

### Example 2

### Corn Transformation

This example illustrates transformation methods useful in producing a transgenic nucleus in a corn plant cell, and the plants, seeds and pollen produced from a transgenic cell with such a nucleus having an enhanced trait, i.e. enhanced water use efficiency, enhanced cold tolerance, increased yield, enhanced nitrogen use efficiency, enhanced seed protein and enhanced seed oil. A plasmid vector is prepared by cloning the DNA of SEQ ID NO:1 into the gene of interest expression cassette in the base vector for use in corn transformation of corn tissue provided in Example 1, Table 3.

For Agrobacterium-mediated transformation of corn embryo cells corn plants of a readily transformable line are grown in the greenhouse and ears are harvested when the embryos are 1.5 to 2.0 mm in length. Ears are surface sterilized by spraying or soaking the ears in 80% ethanol, followed by air drying. Immature embryos are isolated from individual kernels on surface sterilized ears. Prior to inoculation of maize cells, Agrobacterium cells are grown overnight at room temperature. Immature maize embryo cells are inoculated with Agrobacterium shortly after excision, and incubated at room temperature with Agrobacterium for 5-20 minutes. Immature embryo plant cells are then co-cultured with Agrobacterium for 1 to 3 days at 23° C. in the dark. Co-cultured embryos are transferred to selection media and cultured for approximately two weeks to allow embryogenic callus to develop. Embryogenic callus is transferred to culture medium containing 100 mg/L paromomycin and subcultured at about two week intervals. Transformed plant cells are recovered 6 to 8 weeks after initiation of selection.

For Agrobacterium-mediated transformation of maize callus immature embryos are cultured for approximately 8-21 days after excision to allow callus to develop. Callus is then incubated for about 30 minutes at room temperature with the Agrobacterium suspension, followed by removal of the liquid by aspiration. The callus and Agrobacterium are co-cultured without selection for 3-6 days followed by selection on paromomycin for approximately 6 weeks, with biweekly transfers to fresh media. Paromomycin resistant calli are identified about 6-8 weeks after initiation of selection.

To regenerate transgenic corn plants a callus of transgenic plant cells resulting from transformation and selection is placed on media to initiate shoot development into plantlets which are transferred to potting soil for initial growth in a growth chamber at 26° C. followed by a mist bench before transplanting to 5 inch pots where plants are grown to maturity. The regenerated plants are self-fertilized and seed is harvested for use in one or more methods to select seeds, seedlings or progeny second generation transgenic plants (R2 plants) or hybrids, e.g. by selecting transgenic plants exhibiting an enhanced trait as compared to a control plant.

The above process is repeated to produce multiple events of transgenic corn plant cells that are transformed with recombinant DNA from each of the genes identified in Table 1. Events are designed to produce in the transgenic cells one of the proteins identified in Table 1, except the proteins of SEQ ID NOs: 141-144, 152, 174, 190, 203, 208, and 239-240, and the corn homolog of SEQ ID NOs: 122, 188, and 241 which are suppressed. Progeny transgenic plants and seed of the transformed plant cells are screened for enhanced water use efficiency, enhanced cold tolerance, increased yield, enhanced nitrogen use efficiency, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 3

### Soybean Transformation

This example illustrates plant transformation useful in producing a transgenic nucleus in a soybean plant cell, and the plants, seeds and pollen produced from a transgenic cell with such a nucleus having an enhanced trait, i.e. enhanced water use efficiency, enhanced cold tolerance, increased yield, enhanced nitrogen use efficiency, enhanced seed protein and enhanced seed oil.

For Agrobacterium mediated transformation, soybean seeds are imbided overnight and the meristem explants excised. The explants are placed in a wounding vessel. Soybean explants and induced Agrobacterium cells from a strain containing plasmid DNA with the gene of interest cassette and a plant selectable marker cassette are mixed no later than 14 hours from the time of initiation of seed imbibition, and wounded using sonication. Following wounding, explants are placed in co-culture for 2-5 days at which point they are transferred to selection media for 6-8 weeks to allow selection and growth of transgenic shoots. Resistant shoots are harvested approximately 6-8 weeks and placed into selective rooting media for 2-3 weeks. Shoots producing roots are transferred to the greenhouse and potted in soil. Shoots that remain healthy on selection, but do not produce roots are transferred to non-selective rooting media for an additional two weeks. Roots from any shoots that produce roots off selection are tested for expression of the plant selectable marker before they are transferred to the greenhouse and potted in soil.

The above process is repeated to produce multiple events of transgenic soybean plant cells that are transformed with recombinant DNA from each of the genes identified in Table 1. Events are designed to produce in the transgenic cells one of the proteins identified in Table 1, except the proteins of SEQ ID NOs: 122, 188, and 241 and the soybean homologs of SEQ ID NOs: 141-144, 152, 174, 190, 203, 208, and 239-240, which are suppressed. Progeny transgenic plants and seed of the transformed plant cells are screened for enhanced water use efficiency, enhanced cold tolerance, increased yield, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 4

### Cotton Transgenic Plants with Enhanced Agronomic Traits

This example illustrates plant transformation useful in producing a transgenic nucleus in a cotton plant cell, and the plants, seeds and pollen produced from a transgenic cell with such a nucleus having an enhanced trait, i.e. enhanced water use efficiency, increased yield, enhanced nitrogen use efficiency and enhanced seed oil.

Transgenic cotton plants containing each recombinant DNA having a sequence of SEQ ID NO: 1 through SEQ ID NO: 121 are obtained by transforming with recombinant DNA from each of the genes identified in Table 1 using Agrobacterium-mediated transformation. The above process is repeated to produce multiple events of transgenic cotton plant cells that are transformed with recombinant DNA from each of the genes identified in Table 1. Events are designed to produce in the transgenic cells one of the proteins identified in Table 1, except the cotton homologs of the proteins of SEQ ID NOs: 122, 141-144, 152, 174, 188, 190, 203, 208, and 239-241 which are suppressed.

From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

Progeny transgenic plants are selected from a population of transgenic cotton events under specified growing conditions and are compared with control cotton plants. Control cotton plants are substantially the same cotton genotype but without the recombinant DNA, for example, either a parental cotton plant of the same genotype that was not transformed with the identical recombinant DNA or a negative isoline of the transformed plant. Additionally, a commercial cotton cultivar adapted to the geographical region and cultivation conditions, i.e. cotton variety ST474, cotton variety FM 958, and cotton variety Siokra L-23, are used to compare the relative performance of the transgenic cotton plants containing the recombinant DNA.

Transgenic cotton plants with enhanced yield and water use efficiency are identified by growing under variable water conditions. Specific conditions for cotton include growing a first set of transgenic and control plants under “wet” conditions, i.e. irrigated in the range of 85 to 100 percent of evapotranspiration to provide leaf water potential of −14 to −18 bars, and growing a second set of transgenic and control plants under “dry” conditions, i.e. irrigated in the range of 40 to 60 percent of evapotranspiration to provide a leaf water potential of −21 to −25 bars. Pest control, such as weed and insect control is applied equally to both wet and dry treatments as needed. Data gathered during the trial includes weather records throughout the growing season including detailed records of rainfall; soil characterization information; any herbicide or insecticide applications; any gross agronomic differences observed such as leaf morphology, branching habit, leaf color, time to flowering, and fruiting pattern; plant height at various points during the trial; stand density; node and fruit number including node above white flower and node above crack boll measurements; and visual wilt scoring. Cotton boll samples are taken and analyzed for lint fraction and fiber quality. The cotton is harvested at the normal harvest timeframe for the trial area. Enhanced water use efficiency is indicated by increased yield, improved relative water content, enhanced leaf water potential, increased biomass, enhanced leaf extension rates, and improved fiber parameters.

### Example 5

### Canola Transformation

This example illustrates plant transformation useful in producing the transgenic canola plants of this invention and the production and identification of transgenic seed for transgenic canola having enhanced water use efficiency, enhanced cold tolerance, increased yield, enhanced nitrogen use efficiency, enhanced seed protein and enhanced seed oil.

Tissues from in vitro grown canola seedlings are prepared and inoculated with overnight-grown Agrobacterium cells containing plasmid DNA with the gene of interest cassette and a plant selectable marker cassette. Following co-cultivation with Agrobacterium, the infected tissues are allowed to grow on selection to promote growth of transgenic shoots, followed by growth of roots from the transgenic shoots. The selected plantlets are then transferred to the greenhouse and potted in soil. Molecular characterizations are performed to confirm the presence of the gene of interest, and its expression in transgenic plants and progenies. Progeny transgenic plants are selected from a population of transgenic canola events under specified growing conditions and are compared with control canola plants. Control canola plants are substantially the same canola genotype but without the recombinant DNA, for example, either a parental canola plant of the same genotype that is not transformed with the identical recombinant DNA or a negative isoline of the transformed plant.

Transgenic canola plant cells are transformed with each of the recombinant DNA identified in Table 1. The above process is repeated to produce multiple events of transgenic canola plant cells that are transformed with recombinant DNA from each of the genes identified in Table 1. Events are designed to produce in the transgenic cells one of the proteins identified in Table 1, except the canola homologs of the proteins of SEQ ID NOs: 122, 141-144, 152, 174, 188, 190, 203, 208, and 239-241 which are suppressed. Progeny transgenic plants and seed of the transformed plant cells are screened for enhanced water use efficiency, enhanced cold tolerance, increased yield, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 6

### Homolog Identification

This example illustrates the identification of homologs of proteins encoded by the DNA identified in Table 1 which is used to provide transgenic seed and plants having enhanced agronomic traits. From the sequence of the homologs, homologous DNA sequence can be identified for preparing additional transgenic seeds and plants of this invention with enhanced agronomic traits.

An “All Protein Database” was constructed of known protein sequences using a proprietary sequence database and the National Center for Biotechnology Information (NCBI) non-redundant amino acid database (nr.aa). For each organism from which a polynucleotide sequence provided herein was obtained, an “Organism Protein Database” was constructed of known protein sequences of the organism; it is a subset of the All Protein Database based on the NCBI taxonomy ID for the organism.

The All Protein Database was queried using amino acid sequences provided herein as SEQ ID NO: 122 through SEQ ID NO: 242 using NCBI “blastp” program with E-value cutoff of 1e-8. Up to 1000 top hits were kept, and separated by organism names. For each organism other than that of the query sequence, a list was kept for hits from the query organism itself with a more significant E-value than the best hit of the organism. The list contains likely duplicated genes of the polynucleotides provided herein, and is referred to as the Core List. Another list was kept for all the hits from each organism, sorted by E-value, and referred to as the Hit List.

The Organism Protein Database was queried using polypeptide sequences provided herein as SEQ ID NO: 122 through SEQ ID NO: 242 using NCBI “blastp” program with E-value cutoff of 1e-4. Up to 1000 top hits were kept. A BLAST searchable database was constructed based on these hits, and is referred to as “SubDB”. SubDB is queried with each sequence in the Hit List using NCBI “blastp” program with E-value cutoff of 1e-8. The hit with the best E-value was compared with the Core List from the corresponding organism. The hit is deemed a likely ortholog if it belongs to the Core List, otherwise it is deemed not a likely ortholog and there is no further search of sequences in the Hit List for the same organism. Homologs from a large number of distinct organisms were identified and are reported below in table 7 with the SEQ ID NO of the original query sequence and the identified homologs as [SEQ ID NO]: [Homolog SEQ ID NOs].

TABLE 7
Protein Sequences and their Homologs

| | |
|---|---|
| 122: | 244 270 291 322 334 352 364 391 406 427 431 433 456 459 468 470 482 541 570 577 593 599 640 651 654 |
| | 672 676 714 748 761 781 782 789 796 798 799 831 833 857 864 885 891 898 902 904 911 933 955 960 970 985 |
| | 990 1005 1026 1033 1034 1044 1056 1065 1069 1075 1127 1133 1136 |
| 123: | 272 315 328 345 360 414 430 447 460 464 476 561 565 566 591 633 636 650 662 699 703 708 710 725 755 |
| | 765 819 826 828 836 852 866 883 909 923 932 996 1040 1111 1126 1128 1129 1130 1140 1144 1171 |
| 124: | 269 320 321 324 336 358 370 377 384 387 408 420 429 440 445 465 477 488 502 528 586 598 600 606 628 |
| | 663 667 697 698 702 706 745 791 805 806 859 913 931 934 952 953 1046 1070 1097 1099 1102 1113 1118 1120 |
| | 1122 1147 1151 1157 1161 1162 1168 |
| 125: | 249 258 263 271 286 335 337 338 344 350 351 375 390 402 405 411 426 438 439 449 454 458 493 499 517 |
| | 562 563 571 572 573 589 594 603 624 627 631 718 720 739 768 772 776 784 788 793 837 886 889 896 907 925 |
| | 939 943 944 950 957 963 964 969 976 1002 1008 1010 1014 1035 1039 1074 1083 1098 1104 1137 1146 1170 |
| | 1177 1180 |
| 126: | 243 245 246 247 248 250 252 253 254 255 256 257 259 260 261 262 264 265 266 267 268 273 274 276 277 |
| | 278 279 280 282 284 285 287 288 290 292 293 295 296 297 298 300 301 302 303 304 305 306 307 308 309 310 |
| | 311 314 316 317 318 319 323 325 326 327 330 331 332 333 339 340 341 342 343 346 348 349 354 355 356 357 |
| | 359 362 363 365 368 369 371 372 373 374 376 378 379 380 381 382 383 388 389 392 393 394 395 396 398 399 |
| | 400 403 404 407 409 410 413 415 417 418 419 421 422 423 425 432 435 436 441 442 443 446 448 451 452 453 |
| | 455 461 462 463 466 467 469 471 472 473 474 475 479 481 483 484 485 486 487 491 492 495 496 497 498 500 |
| | 501 504 505 506 507 508 509 510 511 512 513 514 515 516 518 522 523 524 525 526 527 529 531 532 533 534 |
| | 536 537 538 539 540 542 543 544 545 546 547 548 549 550 551 553 554 555 556 557 558 559 564 567 568 569 |
| | 575 576 579 580 581 582 583 584 585 587 588 592 595 596 597 602 604 607 608 610 611 613 614 615 616 619 |
| | 620 622 623 625 629 632 634 635 637 639 643 644 647 648 649 652 653 655 656 658 659 660 661 664 666 668 |
| | 669 670 671 673 674 675 677 678 679 680 681 682 683 684 685 686 687 688 689 690 692 693 694 695 696 700 |
| | 701 705 707 709 711 713 715 717 719 722 723 726 728 729 730 731 732 733 734 735 737 738 740 741 742 743 |
| | 746 747 749 750 751 752 753 754 757 758 759 762 763 764 767 769 770 771 773 774 775 777 778 779 780 785 |
| | 786 787 790 792 794 800 801 802 803 804 807 808 810 811 812 813 814 815 816 818 820 821 822 823 824 825 |
| | 827 829 830 832 834 835 838 839 840 841 842 843 844 845 846 847 848 849 850 853 854 855 856 858 860 861 |
| | 862 863 865 867 868 870 871 872 873 874 875 877 878 879 881 882 887 888 890 892 893 894 895 897 899 900 |
| | 901 903 905 906 910 912 914 915 917 918 919 920 921 924 926 927 930 937 938 941 942 946 947 948 949 951 |
| | 954 956 958 959 965 966 967 968 971 972 974 975 977 979 981 983 986 987 988 989 991 992 993 994 995 997 |
| | 998 999 1000 1001 1003 1004 1006 1007 1011 1013 1015 1016 1017 1018 1019 1020 1022 1024 1025 1027 1028 |
| | 1029 1030 1031 1032 1036 1037 1041 1042 1043 1045 1047 1048 1049 1052 1053 1054 1055 1057 1058 1059 |
| | 1060 1061 1062 1064 1067 1068 1073 1077 1078 1079 1080 1081 1082 1084 1085 1086 1087 1088 1089 1090 |
| | 1091 1093 1094 1095 1096 1100 1101 1103 1105 1106 1108 1109 1110 1112 1114 1115 1116 1117 1119 1121 |
| | 1123 1124 1125 1131 1135 1138 1139 1141 1142 1143 1145 1148 1149 1150 1152 1153 1154 1155 1156 1158 |
| | 1159 1160 1163 1164 1165 1166 1167 1172 1173 1174 1175 1176 1178 1179 |
| 127: | 251 275 281 289 294 312 313 329 347 353 361 366 367 386 397 401 424 428 434 437 450 457 478 480 489 |
| | 490 494 503 519 520 521 530 535 574 578 601 605 618 641 657 665 704 712 721 727 744 760 766 783 795 797 |
| | 809 817 851 876 880 884 916 922 928 961 962 973 1009 1021 1023 1038 1050 1051 1063 1066 1072 1076 1092 |
| | 1107 1169 |
| 128: | 283 299 385 412 416 444 552 560 590 609 612 617 621 626 630 638 642 645 646 691 716 724 736 756 869 |
| | 908 929 935 936 940 945 978 980 982 984 1012 1071 1132 1134 |
| 129: | 1188 1200 1204 1214 1232 1237 1273 1287 1291 1296 1313 1327 1334 1337 1338 1344 1369 1383 1418 |
| | 1436 1444 1446 1451 1462 1473 1510 1536 1618 1633 1642 1708 1736 1750 1753 1767 1793 1798 1804 1864 |
| | 1897 1902 1905 1931 1957 1958 1963 1966 1973 1986 2001 2022 2032 2041 2043 2095 2125 2146 2150 2163 |
| | 2204 2211 2214 2252 2261 2305 2314 2315 2319 2328 2347 2348 2350 2352 2353 2354 2363 2408 2440 2445 |
| | 2473 2480 2487 2511 2528 2566 2589 2610 2650 2654 2661 2685 2718 2737 2766 2775 2782 2802 2817 2844 |
| | 2851 2863 2880 2895 2897 2907 2930 2941 2946 |
| 130: | 1215 1219 1222 1270 1349 1358 1360 1406 1474 1530 1560 1562 1570 1597 1612 1650 1669 1691 1723 |
| | 1727 1742 1745 1797 1813 1856 1873 1876 1919 1968 1999 2046 2165 2215 2281 2296 2325 2330 2331 2344 |
| | 2438 2476 2517 2522 2537 2578 2658 2674 2683 2754 2780 2811 2830 2892 2913 2928 |
| 131: | 1182 1206 1208 1243 1253 1278 1285 1286 1292 1299 1301 1345 1412 1489 1492 1503 1582 1588 1620 |
| | 1634 1636 1639 1641 1653 1678 1712 1754 1807 1818 1848 1926 1933 1961 2000 2068 2076 2096 2155 2220 |
| | 2247 2248 2253 2256 2258 2259 2356 2358 2364 2367 2398 2413 2421 2456 2457 2560 2563 2573 2577 2691 |
| | 2762 2816 2825 2842 2847 2854 2874 2920 2923 2950 |
| 132: | 1183 1184 1185 1186 1187 1189 1191 1192 1193 1194 1196 1197 1198 1199 1207 1210 1211 1212 1221 |
| | 1223 1225 1231 1233 1235 1236 1239 1242 1246 1247 1249 1256 1257 1259 1262 1264 1265 1267 1269 1275 |
| | 1277 1284 1289 1295 1297 1298 1300 1304 1305 1306 1308 1310 1311 1312 1314 1315 1319 1321 1322 1323 |
| | 1324 1328 1329 1331 1332 1335 1336 1346 1348 1351 1352 1353 1357 1361 1363 1367 1370 1372 1379 1380 |
| | 1381 1382 1386 1389 1392 1393 1396 1397 1398 1399 1400 1409 1413 1414 1417 1419 1422 1423 1426 1428 |
| | 1430 1431 1432 1437 1440 1442 1445 1456 1457 1459 1460 1463 1465 1466 1468 1469 1472 1475 1476 1477 |
| | 1478 1482 1486 1487 1491 1493 1496 1497 1498 1499 1506 1507 1512 1513 1519 1521 1523 1524 1525 1531 |
| | 1548 1549 1550 1552 1553 1554 1555 1571 1573 1577 1583 1589 1591 1595 1598 1600 1602 1603 1606 1608 |
| | 1613 1623 1626 1627 1629 1630 1631 1640 1649 1651 1652 1663 1665 1666 1668 1671 1673 1679 1680 1682 |
| | 1684 1685 1692 1694 1696 1698 1700 1701 1709 1711 1713 1714 1716 1718 1719 1720 1726 1728 1733 1735 |
| | 1737 1739 1740 1741 1744 1747 1752 1755 1756 1757 1758 1759 1761 1764 1765 1766 1770 1779 1780 1784 |
| | 1786 1794 1795 1799 1801 1802 1803 1806 1808 1811 1814 1822 1823 1825 1827 1828 1829 1832 1837 1839 |
| | 1840 1841 1842 1845 1846 1849 1850 1857 1858 1861 1865 1866 1867 1868 1870 1872 1875 1877 1878 1880 |
| | 1881 1883 1886 1888 1890 1891 1892 1893 1895 1896 1898 1903 1906 1907 1909 1912 1913 1914 1916 1917 |
| | 1918 1923 1925 1927 1928 1930 1935 1937 1940 1941 1942 1944 1945 1947 1948 1949 1950 1951 1956 1959 |
| | 1960 1962 1964 1965 1967 1969 1971 1974 1976 1977 1980 1983 1984 1988 1990 1992 1993 1994 1995 1997 |
| | 2006 2008 2009 2010 2013 2014 2015 2017 2020 2021 2023 2024 2026 2028 2033 2036 2038 2039 2045 2048 |
| | 2050 2053 2055 2060 2061 2063 2064 2065 2069 2077 2081 2082 2084 2085 2087 2088 2090 2091 2092 2094 |
| | 2098 2100 2101 2102 2104 2109 2116 2120 2121 2124 2126 2128 2130 2131 2132 2134 2135 2136 2138 2144 |
| | 2152 2158 2159 2160 2161 2164 2167 2168 2173 2174 2175 2176 2177 2178 2181 2184 2185 2186 2187 2188 |
| | 2189 2190 2198 2201 2205 2216 2218 2219 2221 2222 2228 2232 2233 2234 2235 2236 2238 2240 2241 2243 |
| | 2250 2251 2255 2263 2268 2269 2270 2272 2274 2276 2277 2280 2283 2284 2285 2286 2288 2289 2290 2297 |
| | 2299 2301 2304 2306 2307 2308 2311 2316 2318 2324 2326 2327 2329 2332 2333 2335 2337 2338 2340 2341 |
| | 2342 2345 2355 2359 2360 2362 2368 2376 2378 2379 2382 2384 2385 2387 2388 2391 2392 2393 2394 2395 |
| | 2396 2397 2400 2402 2404 2405 2406 2412 2416 2417 2420 2426 2430 2431 2434 2435 2437 2443 2444 2447 |
| | 2455 2458 2460 2467 2468 2472 2474 2475 2479 2482 2484 2485 2486 2489 2493 2495 2497 2498 2499 2503 |
| | 2504 2506 2507 2508 2510 2514 2518 2520 2521 2525 2527 2530 2534 2536 2541 2542 2545 2546 2549 2554 |
| | 2555 2557 2568 2576 2581 2582 2583 2585 2587 2588 2590 2591 2593 2594 2595 2596 2598 2599 2601 2602 |
| | 2606 2608 2611 2614 2617 2620 2624 2630 2633 2636 2638 2639 2641 2642 2643 2646 2651 2657 2659 2660 |
| | 2664 2671 2676 2680 2681 2682 2684 2687 2689 2692 2694 2695 2696 2697 2698 2700 2701 2703 2714 2715 |
| | 2730 2744 2745 2751 2756 2758 2760 2769 2771 2774 2778 2779 2783 2786 2789 2790 2792 2793 2794 2796 |
| | 2798 2799 2804 2807 2809 2812 2813 2814 2822 2824 2828 2831 2836 2837 2838 2839 2841 2843 2855 2856 |
| | 2857 2858 2860 2861 2868 2872 2873 2877 2878 2881 2882 2884 2888 2891 2894 2899 2900 2901 2905 2906 |
| | 2909 2910 2912 2914 2916 2917 2919 2922 2925 2926 2927 2931 2932 2933 2934 2935 2936 2939 2940 2942 |
| | 2947 2949 |
| 133: | 1317 1330 1343 1390 1441 1494 1579 1777 1830 1855 1871 1934 1938 2089 2271 2312 2538 2693 2723 |
| | 2827 2875 2908 |
| 134: | 1181 1190 1195 1201 1202 1203 1205 1209 1216 1217 1218 1220 1224 1227 1228 1230 1234 1238 1248 |
| | 1255 1258 1260 1261 1268 1272 1274 1276 1279 1280 1282 1283 1288 1302 1303 1307 1316 1318 1320 1325 |
| | 1326 1339 1340 1341 1342 1347 1350 1354 1355 1365 1371 1375 1377 1378 1387 1391 1394 1395 1401 1404 |
| | 1407 1410 1411 1416 1420 1421 1425 1427 1435 1438 1439 1448 1449 1452 1453 1455 1458 1461 1470 1471 |
| | 1479 1500 1502 1505 1508 1509 1511 1515 1518 1520 1522 1526 1527 1528 1529 1535 1539 1540 1542 1545 |
| | 1546 1547 1551 1559 1561 1565 1567 1568 1569 1572 1578 1580 1581 1586 1593 1596 1599 1601 1607 1609 |
| | 1614 1615 1617 1622 1628 1635 1638 1646 1647 1661 1664 1667 1672 1686 1687 1688 1689 1690 1693 1702 |
| | 1703 1704 1705 1717 1721 1722 1725 1729 1730 1731 1732 1738 1746 1749 1760 1762 1763 1768 1769 1771 |
| | 1772 1774 1776 1778 1781 1783 1787 1789 1790 1800 1809 1815 1816 1817 1819 1820 1821 1824 1826 1833 |
| | 1834 1835 1836 1843 1844 1847 1854 1859 1860 1869 1874 1879 1884 1885 1889 1894 1900 1901 1904 1910 |
| | 1911 1921 1922 1929 1936 1943 1946 1954 1955 1970 1975 1979 1981 1982 1989 1998 2004 2005 2016 2018 |
| | 2025 2027 2029 2030 2034 2035 2042 2047 2049 2051 2052 2056 2057 2059 2062 2066 2067 2070 2071 2073 |
| | 2079 2080 2097 2099 2106 2108 2110 2111 2112 2114 2115 2118 2119 2122 2123 2127 2129 2133 2137 2139 |
| | 2140 2141 2142 2143 2145 2147 2148 2151 2153 2156 2157 2166 2169 2170 2171 2180 2182 2191 2192 2193 |
| | 2194 2195 2197 2199 2200 2202 2203 2206 2208 2210 2212 2217 2224 2237 2239 2242 2249 2257 2262 2264 |
| | 2265 2267 2273 2275 2278 2282 2287 2292 2293 2294 2295 2303 2309 2310 2313 2317 2321 2323 2334 2336 |
| | 2339 2343 2351 2357 2361 2365 2371 2372 2373 2374 2375 2380 2381 2386 2399 2409 2410 2414 2415 2419 |
| | 2422 2423 2427 2428 2429 2441 2442 2449 2453 2461 2462 2465 2469 2470 2481 2488 2491 2492 2496 2500 |
| | 2501 2502 2505 2509 2512 2515 2523 2529 2531 2535 2539 2540 2543 2548 2550 2551 2559 2561 2562 2564 |
| | 2567 2569 2571 2572 2574 2575 2579 2580 2586 2597 2605 2609 2612 2615 2616 2622 2623 2627 2628 2629 |
| | 2632 2634 2640 2644 2647 2648 2652 2656 2662 2663 2666 2668 2669 2670 2672 2678 2686 2688 2702 2704 |
| | 2710 2713 2717 2719 2721 2732 2733 2735 2736 2738 2739 2740 2741 2742 2743 2747 2752 2753 2755 2757 |
| | 2761 2763 2770 2772 2776 2784 2788 2791 2795 2797 2800 2801 2806 2815 2818 2819 2820 2821 2823 2832 |
| | 2833 2834 2840 2845 2846 2849 2852 2862 2865 2867 2869 2870 2871 2883 2885 2886 2887 2890 2902 2903 |
| | 2911 2918 2921 2937 2938 2943 2948 |
| 135: | 1250 1366 1484 1514 1674 1676 1681 2074 2075 2425 2432 2436 2653 2655 2896 2898 2944 |
| 136: | 1251 1385 1447 1454 1594 1662 1734 1915 2105 2207 2213 2266 2302 2369 2519 2544 2731 2826 |
| 137: | 1429 1501 1838 2011 2113 2320 2494 2603 2631 2720 2773 2835 |
| 138: | 1240 1402 1415 1654 1683 1706 1707 2007 2040 2196 2300 2322 2366 2618 |
| 139: | 1226 1244 1263 1281 1374 1481 1495 1516 1532 1534 1537 1538 1541 1557 1558 1564 1566 1575 1576 |
| | 1587 1590 1592 1604 1605 1616 1619 1621 1624 1625 1643 1644 1645 1648 1656 1658 1659 1660 1670 1748 |
| | 1791 1792 1924 1953 2078 2086 2107 2117 2223 2246 2349 2390 2403 2433 2450 2463 2471 2553 2556 2558 |
| | 2607 2673 2706 2708 2785 2787 2879 2893 |
| 140: | 1271 1356 1359 1362 1373 1467 1480 1490 1611 1637 1796 1920 1932 1987 2037 2592 2649 2767 2768 |
| | 2781 |
| 141: | 1252 1254 1266 1464 1574 1584 1697 1775 1788 1978 1985 2058 2279 2346 2383 2454 2478 2524 2626 |
| | 2805 2829 2853 |
| 142: | 1293 1434 1563 1610 1632 1657 1699 1724 1853 1882 1887 2003 2031 2162 2377 2439 2446 2451 2483 |
| | 2565 2621 2645 2677 2679 2859 2876 2904 2924 |
| 143: | 1252 1254 1266 1464 1483 1675 1775 1788 1978 1985 2058 2149 2254 2279 2346 2383 2454 2478 2524 |
| | 2552 2626 2734 2805 2829 2853 2945 |
| 145: | 1206 1213 1229 1241 1245 1253 1278 1285 1286 1290 1292 1294 1299 1301 1309 1333 1345 1364 1368 |
| | 1376 1384 1388 1403 1405 1408 1424 1433 1443 1450 1485 1488 1503 1504 1517 1533 1543 1544 1556 1585 |
| | 1620 1634 1636 1639 1641 1655 1677 1678 1695 1710 1712 1715 1743 1751 1754 1773 1782 1785 1805 1807 |
| | 1810 1812 1818 1831 1851 1852 1862 1863 1899 1908 1926 1939 1952 1961 1972 1991 1996 2000 2002 2012 |
| | 2019 2044 2054 2068 2072 2076 2083 2093 2096 2103 2154 2172 2179 2183 2209 2220 2225 2226 2227 2229 |
| | 2230 2231 2244 2245 2247 2248 2258 2259 2260 2291 2298 2356 2358 2370 2389 2398 2401 2407 2411 2413 |
| | 2418 2424 2448 2452 2456 2457 2459 2464 2466 2477 2490 2513 2516 2526 2532 2533 2547 2570 2573 2577 |
| | 2584 2600 2604 2613 2619 2625 2635 2637 2665 2667 2675 2690 2699 2705 2707 2709 2711 2712 2716 2722 |
| | 2724 2725 2726 2727 2728 2729 2746 2748 2749 2750 2759 2762 2764 2765 2777 2803 2808 2810 2816 2825 |
| | 2847 2848 2850 2854 2864 2866 2889 2915 2920 2923 2929 2950 |
| 146: | 3010 3133 3287 3327 3549 3567 3628 3733 3789 3892 3929 4063 4144 4216 4330 4384 4491 4534 4650 |
| | 4713 4736 4773 4954 5075 5162 5209 5404 5606 5885 6115 6134 6136 6207 6223 6384 6399 6441 6443 6636 |
| | 6694 |
| 147: | 3076 3208 3338 3346 3398 3515 3721 3748 3749 3795 3976 4103 4110 4261 4280 4316 4367 4386 4393 |
| | 4416 4417 4460 4518 4595 4664 4670 4755 4791 4949 4982 5027 5032 5088 5125 5163 5193 5239 5255 5295 |
| | 5320 5347 5353 5392 5545 5670 5920 5960 6074 6108 6120 6244 6261 6342 6356 6375 6388 6408 6504 6555 |
| | 6661 6696 6827 6859 6869 6877 |
| 148: | 2975 2981 2983 3034 3103 3135 3138 3141 3145 3147 3198 3220 3256 3283 3311 3391 3400 3442 3485 |
| | 3498 3508 3511 3514 3516 3520 3523 3545 3558 3649 3678 3679 3690 3704 3715 3746 3786 3806 3837 3840 |
| | 3946 3964 3966 3981 3987 4000 4040 4068 4095 4114 4119 4125 4192 4199 4225 4230 4242 4252 4256 4258 |
| | 4268 4325 4354 4356 4407 4477 4498 4535 4548 4551 4553 4555 4577 4693 4718 4749 4752 4783 4811 4835 |
| | 4860 4879 4893 4900 4914 5046 5048 5068 5077 5081 5084 5095 5105 5139 5141 5150 5158 5174 5178 5216 |
| | 5217 5246 5259 5262 5297 5298 5362 5411 5429 5465 5475 5499 5537 5565 5567 5570 5621 5663 5665 5688 |
| | 5689 5700 5724 5733 5738 5761 5801 5828 5855 5912 5925 5957 5959 5987 5990 5997 5998 6001 6005 6015 |
| | 6016 6017 6021 6024 6027 6031 6035 6049 6060 6130 6189 6193 6198 6200 6246 6277 6285 6348 6351 6366 |
| | 6367 6371 6401 6431 6436 6439 6450 6476 6492 6576 6580 6610 6616 6627 6637 6662 6697 6698 6735 6758 |
| | 6785 6794 6806 6807 6812 6821 6822 6830 6889 |
| 149: | 2957 2958 2962 2965 2968 2976 2977 2979 2984 2987 2988 2989 2998 2999 3014 3015 3016 3021 3023 |
| | 3024 3026 3033 3035 3037 3045 3051 3060 3070 3075 3080 3081 3082 3092 3094 3096 3106 3107 3113 3115 |
| | 3121 3123 3125 3127 3132 3134 3140 3150 3151 3156 3157 3159 3161 3173 3176 3179 3190 3193 3196 3204 |
| | 3205 3221 3229 3231 3232 3234 3238 3239 3242 3243 3250 3262 3263 3264 3266 3269 3270 3272 3273 3274 |
| | 3275 3277 3286 3294 3300 3303 3304 3305 3319 3320 3321 3322 3323 3324 3331 3335 3337 3342 3355 3362 |
| | 3363 3371 3372 3374 3375 3378 3380 3382 3383 3384 3386 3396 3409 3410 3411 3417 3418 3420 3437 3456 |
| | 3470 3472 3473 3482 3489 3496 3499 3500 3503 3504 3505 3506 3528 3530 3532 3535 3536 3540 3546 3548 |
| | 3550 3554 3555 3560 3561 3566 3569 3570 3571 3573 3574 3575 3590 3591 3592 3595 3597 3598 3599 3625 |
| | 3627 3630 3632 3634 3651 3652 3661 3668 3688 3700 3710 3712 3719 3724 3726 3739 3740 3741 3754 3755 |
| | 3756 3760 3762 3768 3771 3774 3787 3790 3805 3807 3814 3818 3820 3829 3830 3836 3838 3839 3843 3844 |
| | 3845 3860 3863 3872 3876 3878 3881 3886 3893 3897 3912 3915 3921 3923 3931 3940 3942 3943 3944 3947 |
| | 3950 3968 3982 3984 3989 3990 3994 4002 4003 4004 4009 4012 4021 4025 4030 4031 4041 4042 4045 4046 |
| | 4047 4050 4052 4054 4061 4062 4064 4065 4073 4074 4076 4077 4079 4080 4087 4088 4089 4091 4098 4101 |
| | 4104 4106 4124 4131 4133 4145 4146 4154 4168 4169 4176 4187 4194 4196 4198 4206 4211 4212 4222 4231 |
| | 4234 4236 4238 4241 4244 4279 4288 4291 4293 4298 4301 4305 4306 4307 4315 4320 4324 4334 4335 4338 |
| | 4340 4344 4346 4353 4357 4358 4359 4360 4368 4373 4379 4381 4390 4400 4405 4410 4420 4425 4436 4438 |
| | 4440 4447 4450 4454 4458 4461 4464 4468 4490 4492 4503 4504 4505 4506 4508 4511 4512 4514 4515 4516 |
| | 4523 4526 4533 4538 4541 4564 4585 4599 4600 4601 4605 4611 4614 4623 4625 4628 4636 4637 4649 4652 |
| | 4654 4665 4672 4675 4683 4692 4694 4695 4703 4705 4707 4716 4719 4768 4775 4777 4789 4792 4793 4800 |
| | 4805 4806 4815 4816 4817 4819 4823 4825 4834 4864 4866 4875 4889 4891 4903 4908 4917 4923 4925 4933 |
| | 4936 4937 4953 4962 4963 4965 4966 4986 4987 4988 4989 4990 4999 5000 5010 5011 5014 5015 5019 5022 |
| | 5024 5028 5059 5061 5062 5063 5066 5082 5085 5092 5093 5099 5101 5110 5111 5112 5126 5148 5167 5179 |
| | 5181 5184 5185 5188 5189 5194 5219 5224 5237 5247 5252 5256 5257 5283 5285 5293 5296 5299 5301 5303 |
| | 5304 5317 5330 5338 5341 5342 5343 5345 5349 5351 5354 5355 5369 5378 5384 5385 5394 5399 5413 5414 |
| | 5420 5422 5430 5431 5432 5436 5438 5445 5446 5447 5448 5449 5453 5454 5455 5456 5459 5460 5477 5501 |
| | 5503 5511 5516 5517 5518 5520 5540 5541 5549 5550 5566 5568 5569 5573 5579 5582 5584 5585 5591 5592 |
| | 5594 5596 5597 5607 5608 5614 5617 5619 5620 5624 5627 5631 5632 5636 5637 5638 5640 5650 5658 5659 |
| | 5661 5662 5666 5682 5683 5690 5693 5694 5714 5719 5721 5742 5744 5745 5746 5748 5750 5753 5760 5768 |
| | 5769 5772 5775 5776 5777 5778 5781 5782 5783 5785 5786 5794 5802 5805 5810 5812 5814 5817 5839 5849 |
| | 5850 5857 5861 5862 5879 5888 5890 5893 5898 5902 5903 5908 5910 5928 5930 5932 5948 5969 5970 5971 |
| | 5973 5982 5986 5993 5996 6012 6014 6029 6033 6034 6042 6043 6044 6047 6056 6061 6064 6076 6082 6083 |
| | 6087 6095 6096 6103 6105 6107 6109 6110 6111 6114 6116 6123 6125 6139 6141 6143 6144 6147 6148 6150 |
| | 6153 6156 6173 6174 6208 6213 6231 6232 6235 6241 6242 6245 6249 6251 6252 6253 6254 6256 6269 6270 |
| | 6291 6293 6299 6302 6321 6322 6330 6332 6334 6337 6338 6341 6347 6359 6363 6368 6370 6373 6383 6395 |
| | 6398 6400 6409 6424 6425 6426 6429 6438 6446 6484 6485 6503 6507 6510 6515 6517 6547 6550 6554 6572 |
| | 6573 6575 6583 6586 6587 6591 6595 6598 6612 6617 6626 6631 6633 6650 6664 6665 6666 6668 6686 6689 |
| | 6699 6705 6706 6710 6714 6715 6716 6717 6721 6725 6730 6756 6760 6761 6764 6767 6772 6774 6775 6780 |
| | 6781 6809 6810 6824 6826 6832 6834 6835 6848 6849 6851 6862 6864 6866 6868 6879 6883 6892 6896 6900 |
| | 6901 6902 |
| 150: | 2957 2958 2962 2965 2968 2976 2977 2979 2984 2987 2988 2989 2998 2999 3014 3015 3016 3021 3023 |
| | 3024 3026 3033 3035 3037 3045 3051 3056 3060 3070 3075 3080 3081 3082 3092 3094 3096 3106 3107 3115 |
| | 3121 3123 3125 3127 3132 3134 3140 3150 3151 3156 3157 3159 3161 3173 3176 3179 3190 3193 3196 3204 |
| | 3205 3221 3229 3231 3232 3238 3239 3242 3243 3246 3250 3262 3263 3264 3269 3270 3272 3273 3274 3275 |
| | 3277 3286 3300 3303 3304 3310 3321 3322 3323 3324 3331 3335 3337 3342 3355 3362 3363 3371 3372 3374 |
| | 3375 3378 3382 3383 3384 3386 3396 3409 3410 3411 3417 3420 3437 3456 3470 3472 3473 3482 3489 3496 |
| | 3499 3500 3503 3504 3505 3506 3528 3530 3532 3535 3536 3540 3546 3548 3550 3554 3555 3560 3561 3566 |
| | 3568 3569 3570 3571 3573 3574 3591 3592 3595 3597 3598 3599 3625 3627 3630 3632 3634 3651 3652 3661 |
| | 3668 3688 3700 3710 3712 3719 3724 3726 3739 3740 3741 3754 3755 3756 3760 3762 3768 3771 3774 3787 |
| | 3790 3796 3805 3807 3814 3818 3820 3829 3830 3836 3838 3839 3843 3844 3845 3860 3863 3872 3874 3876 |
| | 3878 3881 3886 3893 3894 3897 3912 3915 3921 3931 3940 3943 3944 3947 3968 3982 3984 3989 3990 3994 |
| | 4002 4003 4004 4009 4012 4021 4025 4030 4041 4042 4045 4046 4047 4050 4052 4054 4061 4062 4064 4073 |
| | 4074 4076 4077 4079 4080 4087 4088 4089 4091 4098 4101 4106 4124 4131 4133 4145 4146 4154 4168 4169 |
| | 4176 4187 4194 4196 4198 4206 4211 4212 4222 4231 4234 4236 4238 4241 4244 4279 4287 4288 4291 4293 |
| | 4298 4301 4305 4306 4307 4315 4324 4334 4335 4338 4340 4344 4346 4350 4353 4357 4358 4359 4360 4368 |
| | 4373 4379 4381 4390 4391 4405 4410 4420 4425 4436 4438 4440 4447 4449 4450 4454 4458 4461 4464 4468 |
| | 4482 4485 4490 4492 4503 4504 4505 4506 4508 4511 4512 4514 4515 4516 4517 4523 4526 4533 4538 4541 |
| | 4564 4585 4592 4599 4600 4601 4605 4611 4614 4623 4625 4628 4636 4637 4640 4649 4652 4654 4665 4672 |
| | 4675 4683 4692 4694 4695 4702 4703 4704 4705 4707 4716 4777 4789 4792 4793 4800 4805 4806 4815 4817 |
| | 4819 4823 4825 4834 4852 4864 4866 4875 4889 4903 4908 4917 4923 4930 4933 4936 4937 4953 4962 4963 |
| | 4965 4966 4986 4987 4988 4989 4990 4999 5000 5010 5011 5014 5015 5019 5020 5022 5024 5028 5061 5062 |
| | 5063 5082 5085 5092 5093 5099 5101 5110 5111 5112 5115 5126 5165 5167 5179 5181 5184 5185 5188 5189 |
| | 5194 5219 5221 5224 5237 5247 5252 5256 5257 5283 5285 5293 5296 5299 5301 5303 5304 5317 5324 5330 |
| | 5338 5341 5342 5343 5344 5345 5349 5351 5354 5355 5368 5369 5378 5384 5385 5394 5399 5402 5413 5414 |
| | 5420 5422 5430 5431 5432 5436 5438 5445 5446 5447 5448 5449 5453 5454 5455 5456 5459 5460 5477 5501 |
| | 5503 5507 5511 5516 5517 5518 5520 5541 5546 5549 5555 5566 5568 5569 5573 5579 5582 5584 5585 5591 |
| | 5592 5594 5596 5597 5607 5608 5612 5614 5615 5617 5619 5620 5624 5627 5631 5632 5636 5637 5638 5640 |
| | 5650 5658 5659 5661 5666 5682 5683 5690 5693 5694 5714 5719 5721 5742 5744 5745 5746 5748 5750 5753 |
| | 5760 5768 5769 5772 5775 5776 5777 5778 5781 5782 5783 5785 5786 5794 5802 5805 5810 5811 5812 5814 |
| | 5817 5839 5849 5850 5857 5862 5865 5888 5890 5893 5898 5902 5908 5910 5928 5932 5948 5969 5970 5971 |
| | 5973 5982 5986 5993 5996 6012 6014 6019 6029 6033 6034 6042 6043 6044 6047 6055 6056 6061 6064 6076 |
| | 6082 6083 6085 6087 6095 6096 6103 6105 6107 6109 6110 6111 6114 6116 6121 6123 6125 6139 6141 6142 |
| | 6143 6144 6147 6148 6150 6153 6156 6173 6174 6186 6208 6213 6231 6232 6235 6241 6242 6245 6249 6251 |
| | 6252 6253 6254 6256 6269 6270 6291 6293 6299 6302 6321 6322 6330 6332 6334 6337 6338 6347 6359 6363 |
| | 6368 6370 6373 6383 6395 6398 6400 6409 6424 6425 6426 6438 6446 6484 6485 6503 6510 6515 6517 6547 |
| | 6550 6554 6572 6573 6575 6583 6586 6587 6591 6595 6598 6612 6626 6643 6650 6664 6665 6666 6668 6686 |
| | 6689 6699 6705 6706 6710 6714 6715 6716 6717 6721 6725 6730 6756 6760 6761 6764 6767 6772 6774 6775 |
| | 6780 6781 6809 6810 6824 6826 6832 6834 6835 6846 6848 6849 6851 6862 6864 6866 6868 6879 6883 6892 |
| | 6896 6900 6901 6902 |
| 151: | 2956 2969 3029 3047 3055 3068 3088 3122 3340 3405 3466 3493 3501 3502 3522 3589 3641 3670 3671 |
| | 3681 3810 3812 3852 3884 3909 3928 3937 3952 3978 4020 4129 4220 4343 4345 4349 4418 4466 4480 4489 |
| | 4545 4582 4584 4591 4606 4643 4644 4645 4803 4837 4846 4850 4876 4896 4926 4929 5029 5100 5140 5159 |
| | 5182 5186 5190 5195 5199 5227 5229 5232 5234 5236 5240 5264 5266 5269 5271 5272 5300 5302 5305 5306 |
| | 5309 5327 5334 5336 5337 5339 5374 5379 5386 5389 5390 5613 5628 5652 5695 5712 5756 5872 5896 5909 |
| | 5931 5949 5974 6098 6353 6377 6381 6410 6522 6527 6552 6556 6733 6769 6777 6823 6878 6882 6887 6894 |
| | 6907 6909 |
| 152: | 2956 2969 3029 3047 3055 3088 3122 3340 3405 3466 3493 3501 3502 3522 3589 3641 3670 3671 3681 |
| | 3810 3812 3852 3884 3909 3928 3937 3952 3978 4020 4129 4220 4343 4345 4349 4418 4466 4480 4489 4545 |
| | 4582 4584 4591 4606 4643 4644 4645 4803 4837 4846 4850 4876 4896 4926 4929 5029 5100 5140 5159 5182 |
| | 5186 5190 5195 5199 5227 5229 5232 5234 5236 5240 5264 5266 5269 5271 5272 5300 5302 5305 5306 5309 |
| | 5334 5336 5337 5339 5374 5379 5386 5389 5390 5613 5628 5652 5695 5712 5756 5872 5896 5931 5949 5974 |
| | 6098 6353 6377 6381 6410 6522 6527 6552 6556 6733 6769 6777 6823 6878 6882 6887 6894 6907 6909 |
| 153: | 3031 3299 3413 3527 3541 3633 3674 4424 4853 5574 5847 5929 6324 6404 6423 6495 |
| 154: | 2971 3072 3377 3433 3488 3934 4028 4035 4136 4160 4265 4558 4560 4580 4669 4972 5016 5103 5196 |
| | 5418 5509 5583 5589 5681 5725 5919 5994 6088 6314 6345 6352 6415 6489 6601 6632 6683 6811 6910 |
| 155: | 3095 |
| 156: | 2952 2953 2986 3201 3447 3457 3684 3689 3761 3935 4014 4018 4075 4121 4170 4259 4321 4642 4742 |
| | 4788 4856 4942 4984 5003 5203 5260 5281 5352 5381 5528 5564 5644 5684 5829 5914 6075 6106 6170 6203 |
| | 6331 6448 6493 6499 6599 6624 6659 6673 6731 6734 6750 6802 6820 6841 6880 6891 6903 6916 |
| 157: | 2951 2960 2964 2970 2974 2978 2980 2985 2990 2991 3002 3005 3008 3013 3017 3038 3041 3043 3048 |
| | 3067 3079 3085 3091 3093 3098 3101 3102 3104 3105 3108 3110 3118 3120 3126 3136 3144 3146 3153 3155 |
| | 3162 3164 3167 3170 3172 3175 3178 3183 3184 3185 3191 3195 3200 3206 3213 3217 3237 3249 3253 3257 |
| | 3268 3271 3278 3280 3281 3282 3288 3289 3290 3293 3313 3317 3328 3343 3347 3348 3352 3359 3370 3381 |
| | 3388 3394 3399 3434 3436 3440 3441 3444 3451 3476 3479 3481 3484 3509 3512 3525 3529 3533 3534 3543 |
| | 3544 3557 3563 3579 3583 3584 3594 3604 3605 3611 3615 3616 3617 3618 3621 3622 3624 3635 3637 3638 |
| | 3639 3640 3642 3643 3644 3648 3654 3658 3659 3680 3683 3693 3695 3697 3699 3703 3705 3706 3714 3720 |
| | 3723 3732 3743 3744 3745 3751 3764 3765 3770 3777 3781 3788 3793 3804 3813 3825 3826 3835 3842 3847 |
| | 3855 3856 3857 3865 3866 3867 3869 3871 3882 3890 3896 3899 3907 3914 3927 3932 3939 3954 3960 3971 |
| | 3975 3980 3991 4008 4013 4017 4029 4033 4034 4053 4055 4056 4058 4060 4078 4083 4085 4086 4092 4100 |
| | 4109 4112 4117 4120 4127 4128 4130 4134 4156 4159 4161 4172 4180 4197 4200 4201 4202 4204 4205 4229 |
| | 4237 4240 4262 4263 4267 4269 4270 4271 4272 4274 4277 4283 4290 4292 4295 4297 4314 4328 4329 4337 |
| | 4366 4372 4377 4378 4388 4389 4398 4399 4403 4409 4412 4413 4415 4423 4428 4431 4435 4442 4448 4457 |
| | 4472 4481 4484 4487 4497 4507 4510 4521 4532 4540 4543 4562 4563 4567 4569 4570 4574 4576 4578 4579 |
| | 4593 4610 4624 4626 4627 4630 4631 4632 4634 4635 4648 4656 4658 4662 4667 4668 4671 4674 4678 4681 |
| | 4691 4700 4701 4708 4712 4715 4721 4722 4733 4734 4741 4748 4753 4758 4764 4765 4771 4772 4782 4785 |
| | 4794 4798 4802 4808 4810 4820 4827 4828 4830 4831 4836 4838 4843 4855 4865 4867 4868 4873 4877 4878 |
| | 4880 4883 4885 4895 4911 4913 4915 4918 4920 4921 4924 4943 4947 4951 4957 4959 4973 4974 4977 4985 |
| | 4992 4997 5001 5002 5004 5012 5033 5036 5038 5054 5078 5091 5106 5108 5113 5117 5118 5127 5128 5130 |
| | 5134 5142 5143 5144 5151 5152 5156 5160 5166 5169 5175 5200 5212 5214 5218 5220 5225 5230 5233 5241 |
| | 5243 5244 5248 5253 5268 5275 5276 5277 5282 5312 5313 5318 5319 5331 5333 5340 5357 5361 5364 5365 |
| | 5367 5382 5400 5403 5405 5409 5417 5425 5426 5439 5467 5470 5473 5474 5480 5482 5484 5485 5486 5487 |
| | 5488 5490 5494 5495 5502 5504 5508 5510 5512 5514 5521 5526 5529 5530 5533 5539 5542 5551 5553 5557 |
| | 5560 5561 5563 5577 5587 5593 5598 5600 5601 5618 5629 5634 5639 5645 5646 5647 5648 5656 5664 5669 |
| | 5673 5674 5675 5676 5678 5685 5696 5697 5701 5704 5705 5710 5713 5716 5727 5729 5730 5731 5762 5763 |
| | 5773 5780 5784 5787 5809 5813 5818 5826 5831 5832 5835 5836 5837 5838 5841 5842 5845 5851 5853 5860 |
| | 5867 5874 5883 5891 5894 5899 5900 5904 5915 5922 5933 5935 5936 5939 5941 5943 5944 5947 5950 5951 |
| | 5953 5963 5968 5975 5979 5984 5991 5995 5999 6002 6011 6025 6032 6037 6038 6039 6045 6046 6050 6052 |
| | 6065 6068 6069 6078 6080 6094 6102 6117 6124 6128 6131 6135 6137 6138 6154 6160 6164 6168 6172 6183 |
| | 6191 6201 6202 6204 6214 6216 6219 6222 6224 6229 6234 6243 6257 6262 6266 6273 6276 6278 6283 6294 |
| | 6298 6307 6310 6315 6335 6336 6349 6357 6360 6378 6379 6382 6385 6389 6390 6393 6405 6406 6416 6419 |
| | 6427 6430 6432 6433 6434 6435 6451 6455 6456 6458 6469 6470 6471 6473 6474 6479 6481 6482 6483 6486 |
| | 6490 6496 6497 6506 6508 6514 6516 6519 6520 6526 6528 6533 6534 6535 6538 6543 6548 6562 6568 6578 |
| | 6579 6581 6582 6584 6593 6600 6603 6604 6605 6614 6615 6629 6634 6657 6669 6670 6677 6680 6681 6684 |
| | 6688 6690 6691 6711 6727 6728 6747 6752 6765 6766 6770 6773 6776 6778 6782 6795 6805 6808 6813 6838 |
| | 6839 6842 6843 6844 6852 6857 6861 6874 6875 6876 6890 6893 6897 6914 |
| 158: | 3003 3012 3032 3078 3171 3233 3276 3285 3291 3341 3369 3379 3408 3424 3614 3696 3817 3849 3862 |
| | 3880 4016 4032 4084 4140 4148 4174 4286 4310 4319 4336 4483 4557 4573 4687 4709 4790 4872 4894 4899 |
| | 4935 4971 4983 4991 4994 5031 5034 5045 5052 5223 5267 5292 5360 5408 5496 5580 5611 5737 5743 5752 |
| | 5791 5793 5804 5823 5846 5911 5952 6000 6007 6058 6084 6113 6126 6146 6163 6196 6206 6211 6346 6380 |
| | 6420 6428 6500 6524 6525 6546 6559 6635 6685 6700 6712 6720 6788 6801 6831 6840 6885 |
| 159: | 2997 3011 3036 3040 3111 3112 3117 3174 3267 3471 3483 3521 3576 3586 3607 3610 3682 3718 3824 |
| | 3974 4038 4069 4097 4208 4233 4235 4239 4243 4248 4376 4411 4520 4666 4980 5009 5087 5102 5328 5359 |
| | 5372 5544 5588 5635 5871 5907 6028 6054 6112 6177 6205 6295 6319 6326 6413 6444 6445 6609 6663 6732 |
| | 6757 6915 |
| 160: | 2954 2955 2963 2967 2973 2994 3006 3007 3009 3022 3039 3042 3046 3049 3054 3057 3058 3059 3061 |
| | 3064 3069 3083 3086 3087 3090 3097 3099 3100 3114 3129 3130 3131 3137 3149 3152 3154 3160 3163 3166 |
| | 3169 3177 3188 3189 3192 3199 3207 3212 3216 3218 3226 3227 3236 3241 3244 3245 3251 3252 3254 3255 |
| | 3259 3261 3265 3284 3301 3302 3308 3309 3312 3325 3329 3330 3333 3334 3357 3358 3365 3387 3390 3392 |
| | 3393 3395 3404 3412 3415 3422 3423 3425 3428 3429 3439 3459 3460 3461 3463 3465 3467 3468 3475 3477 |
| | 3480 3486 3495 3497 3510 3517 3518 3519 3524 3531 3538 3539 3542 3547 3551 3553 3556 3564 3565 3572 |
| | 3581 3582 3588 3593 3601 3619 3626 3663 3664 3665 3667 3669 3675 3676 3687 3691 3692 3702 3709 3711 |
| | 3717 3722 3725 3727 3729 3736 3738 3742 3750 3753 3766 3767 3773 3775 3780 3783 3785 3791 3792 3798 |
| | 3799 3802 3803 3809 3811 3831 3846 3848 3854 3861 3864 3868 3883 3887 3888 3904 3908 3911 3913 3919 |
| | 3922 3924 3926 3930 3953 3956 3957 3958 3959 3961 3962 3963 3965 3977 3985 3992 3993 3996 3998 3999 |
| | 4001 4005 4006 4010 4011 4022 4023 4024 4037 4039 4043 4044 4051 4057 4059 4067 4072 4081 4090 4093 |
| | 4094 4099 4102 4105 4108 4113 4116 4137 4143 4149 4151 4158 4166 4167 4171 4173 4175 4177 4178 4183 |
| | 4184 4195 4209 4215 4219 4226 4228 4232 4246 4247 4250 4254 4255 4257 4260 4264 4266 4275 4278 4281 |
| | 4282 4294 4296 4300 4302 4303 4304 4318 4322 4331 4339 4342 4348 4361 4364 4365 4375 4382 4383 4385 |
| | 4394 4395 4396 4397 4402 4406 4408 4419 4422 4437 4439 4441 4443 4445 4455 4456 4459 4463 4467 4473 |
| | 4476 4494 4496 4499 4501 4509 4519 4522 4528 4531 4539 4544 4546 4547 4552 4559 4565 4568 4571 4587 |
| | 4588 4589 4590 4594 4603 4604 4608 4609 4612 4613 4618 4620 4639 4641 4647 4653 4655 4661 4673 4677 |
| | 4679 4680 4682 4684 4685 4686 4688 4689 4697 4698 4699 4711 4720 4724 4727 4728 4735 4744 4746 4750 |
| | 4754 4756 4757 4761 4762 4763 4779 4780 4784 4787 4796 4797 4799 4804 4813 4814 4824 4826 4829 4839 |
| | 4840 4841 4842 4845 4848 4849 4854 4857 4859 4862 4863 4870 4871 4882 4886 4888 4890 4898 4902 4906 |
| | 4907 4910 4912 4916 4928 4932 4939 4940 4944 4950 4952 4958 4960 4961 4964 4970 4976 4978 4993 4996 |
| | 5005 5006 5007 5017 5023 5030 5035 5039 5041 5047 5056 5067 5070 5074 5076 5079 5083 5086 5090 5096 |
| | 5109 5114 5136 5137 5146 5154 5164 5171 5177 5191 5192 5202 5205 5206 5207 5213 5215 5226 5228 5250 |
| | 5254 5258 5261 5263 5265 5270 5273 5278 5280 5284 5290 5291 5308 5322 5323 5325 5332 5335 5348 5366 |
| | 5370 5375 5380 5398 5401 5407 5412 5421 5423 5424 5434 5435 5440 5442 5444 5451 5452 5457 5458 5463 |
| | 5466 5468 5469 5472 5479 5492 5505 5513 5519 5522 5524 5525 5535 5548 5552 5556 5572 5586 5590 5603 |
| | 5605 5609 5622 5623 5626 5641 5642 5651 5654 5657 5660 5672 5677 5679 5687 5692 5703 5707 5708 5709 |
| | 5711 5717 5722 5723 5726 5735 5736 5740 5747 5751 5755 5757 5764 5765 5766 5770 5774 5790 5792 5796 |
| | 5798 5803 5806 5808 5816 5820 5824 5830 5843 5844 5848 5854 5864 5869 5873 5876 5889 5892 5897 5906 |
| | 5916 5918 5927 5945 5954 5958 5961 5962 5964 5966 5967 5972 5976 5980 5981 5983 5985 6003 6004 6006 |
| | 6009 6010 6023 6030 6036 6051 6062 6067 6070 6073 6090 6093 6101 6104 6119 6122 6127 6132 6149 6152 |
| | 6159 6176 6181 6182 6194 6197 6218 6221 6227 6228 6230 6233 6239 6240 6248 6250 6255 6260 6263 6267 |
| | 6268 6274 6280 6282 6290 6306 6309 6317 6318 6323 6325 6327 6329 6339 6340 6344 6350 6362 6364 6365 |
| | 6369 6374 6376 6392 6396 6397 6407 6412 6414 6417 6418 6422 6442 6447 6453 6454 6457 6459 6463 6466 |
| | 6467 6475 6477 6480 6487 6501 6502 6511 6513 6529 6531 6532 6540 6553 6560 6561 6569 6570 6571 6577 |
| | 6585 6588 6589 6590 6592 6597 6618 6620 6622 6630 6639 6642 6645 6646 6647 6649 6654 6656 6660 6672 |
| | 6675 6676 6678 6682 6687 6693 6695 6707 6708 6709 6724 6726 6736 6737 6738 6739 6740 6743 6749 6759 |
| | 6763 6783 6787 6790 6791 6797 6798 6799 6800 6814 6815 6817 6828 6837 6845 6853 6855 6858 6863 6865 |
| | 6870 6872 6886 6888 6898 6905 6906 6908 6911 |
| 161: | 2959 2961 2966 2972 2982 2992 2993 2995 2996 3000 3001 3004 3018 3019 3020 3025 3027 3028 3030 |
| | 3044 3050 3052 3053 3062 3063 3065 3066 3071 3073 3074 3077 3084 3089 3109 3116 3119 3124 3128 3139 |
| | 3142 3143 3148 3158 3165 3168 3180 3181 3182 3186 3187 3194 3197 3202 3203 3209 3210 3211 3214 3215 |
| | 3219 3222 3223 3224 3225 3228 3230 3235 3240 3247 3248 3258 3260 3279 3292 3295 3296 3297 3298 3306 |
| | 3307 3314 3315 3316 3318 3326 3332 3336 3339 3344 3345 3349 3350 3351 3353 3354 3356 3360 3361 3364 |
| | 3366 3367 3368 3373 3376 3385 3389 3397 3401 3402 3403 3406 3407 3414 3416 3419 3421 3426 3427 3430 |
| | 3431 3432 3435 3438 3443 3445 3446 3448 3449 3450 3452 3453 3454 3455 3458 3462 3464 3469 3474 3478 |
| | 3487 3490 3491 3492 3494 3507 3513 3526 3537 3552 3559 3562 3577 3578 3580 3585 3587 3596 3600 3602 |
| | 3603 3606 3608 3609 3612 3613 3620 3623 3629 3631 3636 3645 3646 3647 3650 3653 3655 3656 3657 3660 |
| | 3662 3666 3672 3673 3677 3685 3686 3694 3698 3701 3707 3708 3713 3716 3728 3730 3731 3734 3735 3737 |
| | 3747 3752 3757 3758 3759 3763 3769 3772 3776 3778 3779 3782 3784 3794 3797 3800 3801 3808 3815 3816 |
| | 3819 3821 3822 3823 3827 3828 3832 3833 3834 3841 3850 3851 3853 3858 3859 3870 3873 3875 3877 3879 |
| | 3885 3889 3891 3895 3898 3900 3901 3902 3903 3905 3906 3910 3916 3917 3918 3920 3925 3933 3936 3938 |
| | 3941 3945 3948 3949 3951 3955 3967 3969 3970 3972 3973 3979 3983 3986 3988 3995 3997 4007 4015 4019 |
| | 4026 4027 4036 4048 4049 4066 4070 4071 4082 4096 4107 4111 4115 4118 4122 4123 4126 4132 4135 4138 |
| | 4139 4141 4142 4147 4150 4152 4153 4155 4157 4162 4163 4164 4165 4179 4181 4182 4185 4186 4188 4189 |
| | 4190 4191 4193 4203 4207 4210 4213 4214 4217 4218 4221 4223 4224 4227 4245 4249 4251 4253 4273 4276 |
| | 4284 4285 4289 4299 4308 4309 4311 4312 4313 4317 4323 4326 4327 4332 4333 4341 4347 4351 4352 4355 |
| | 4362 4363 4369 4370 4371 4374 4380 4387 4392 4401 4404 4414 4421 4426 4427 4429 4430 4432 4433 4434 |
| | 4444 4446 4451 4452 4453 4462 4465 4469 4470 4471 4474 4475 4478 4479 4486 4488 4493 4495 4500 4502 |
| | 4513 4524 4525 4527 4529 4530 4536 4537 4542 4549 4550 4554 4556 4561 4566 4572 4575 4581 4583 4586 |
| | 4596 4597 4598 4602 4607 4615 4616 4617 4619 4621 4622 4629 4633 4638 4646 4651 4657 4659 4660 4663 |
| | 4676 4690 4696 4706 4710 4714 4717 4723 4725 4726 4729 4730 4731 4732 4737 4738 4739 4740 4743 4745 |
| | 4747 4751 4759 4760 4766 4767 4769 4770 4774 4776 4778 4780 4781 4786 4795 4801 4807 4809 4812 4818 |
| | 4821 4822 4832 4833 4844 4847 4851 4858 4861 4869 4874 4881 4884 4887 4892 4897 4901 4904 4905 4909 |
| | 4919 4922 4927 4931 4934 4938 4941 4945 4946 4948 4955 4956 4967 4968 4969 4975 4979 4981 4995 4998 |
| | 5008 5013 5018 5021 5025 5026 5037 5040 5042 5043 5044 5049 5050 5051 5053 5055 5057 5058 5060 5064 |
| | 5065 5069 5071 5072 5073 5080 5089 5094 5097 5098 5104 5107 5116 5119 5120 5121 5122 5123 5124 5129 |
| | 5131 5132 5133 5135 5138 5145 5147 5149 5153 5155 5157 5161 5168 5170 5172 5173 5176 5180 5183 5187 |
| | 5197 5198 5201 5204 5208 5210 5211 5222 5231 5235 5238 5242 5245 5249 5251 5274 5279 5286 5287 5288 |
| | 5289 5294 5307 5310 5311 5314 5315 5316 5321 5326 5329 5346 5350 5356 5358 5363 5371 5373 5376 5377 |
| | 5383 5387 5388 5391 5393 5395 5396 5397 5406 5410 5415 5416 5419 5427 5428 5433 5437 5441 5443 5450 |
| | 5461 5462 5464 5471 5476 5478 5481 5483 5489 5491 5493 5497 5498 5500 5506 5515 5523 5527 5531 5532 |
| | 5534 5536 5538 5543 5547 5554 5558 5559 5562 5571 5575 5576 5578 5581 5595 5599 5602 5604 5610 5616 |
| | 5625 5630 5633 5643 5649 5653 5655 5667 5668 5671 5680 5686 5691 5698 5699 5702 5706 5715 5718 5720 |
| | 5728 5732 5734 5739 5741 5749 5754 5758 5759 5767 5771 5779 5788 5789 5795 5797 5799 5800 5807 5815 |
| | 5819 5821 5822 5825 5827 5833 5834 5840 5852 5856 5858 5859 5863 5866 5868 5870 5875 5877 5878 5880 |
| | 5881 5882 5884 5886 5887 5895 5901 5905 5913 5917 5921 5923 5924 5926 5934 5937 5938 5940 5942 5946 |
| | 5955 5956 5965 5977 5978 5988 5989 5992 6008 6013 6018 6020 6022 6026 6040 6041 6048 6053 6057 6059 |
| | 6063 6066 6071 6072 6077 6079 6081 6086 6089 6091 6092 6097 6099 6100 6118 6129 6133 6140 6145 6151 |
| | 6155 6157 6158 6161 6162 6165 6166 6167 6169 6171 6175 6178 6179 6180 6184 6185 6187 6188 6190 6192 |
| | 6195 6199 6209 6210 6212 6215 6217 6220 6225 6226 6236 6237 6238 6247 6258 6259 6264 6265 6271 6272 |
| | 6275 6279 6281 6284 6286 6287 6288 6289 6292 6296 6297 6300 6301 6303 6304 6305 6308 6311 6312 6313 |
| | 6316 6320 6328 6333 6343 6354 6355 6358 6361 6372 6386 6387 6391 6394 6402 6403 6411 6421 6437 6440 |
| | 6449 6452 6460 6461 6462 6464 6465 6468 6472 6478 6488 6491 6494 6498 6505 6509 6512 6518 6521 6523 |
| | 6530 6536 6537 6539 6541 6542 6544 6545 6549 6551 6557 6558 6563 6564 6565 6566 6567 6574 6594 6596 |
| | 6602 6606 6607 6608 6611 6613 6619 6621 6623 6625 6628 6638 6640 6641 6644 6648 6651 6652 6653 6655 |
| | 6658 6667 6671 6674 6679 6692 6701 6702 6703 6704 6713 6718 6719 6722 6723 6729 6741 6742 6744 6745 |
| | 6746 6748 6751 6753 6754 6755 6762 6768 6771 6779 6784 6786 6789 6792 6793 6796 6803 6804 6816 6818 |
| | 6819 6825 6829 6833 6836 6847 6850 6854 6856 6860 6867 6871 6873 6881 6884 6895 6899 6904 6912 6913 |
| 162: | 6919 6922 6926 6930 6932 6933 6946 6948 6950 6953 6955 6972 6994 7027 7028 7030 7033 7035 7044 |
| | 7047 7049 7050 7051 7054 7063 7067 7072 7073 7077 7080 7083 7085 7088 7100 7101 7106 7112 7116 7117 |
| | 7118 7124 7128 7129 7130 7132 7134 7136 7137 7138 7150 7153 7154 7156 7157 7175 7338 7368 7394 7396 |
| | 7398 7401 7403 7412 7430 7431 7433 7438 7443 7445 7448 7454 7455 7467 7469 7472 7473 7478 7479 7484 |
| | 7493 7495 7499 7500 7501 7502 7514 7518 7519 7523 7524 7526 7527 7535 7539 7544 7546 7547 7549 7550 |
| | 7552 7553 7560 7562 7578 7585 7671 7680 7682 7692 7698 7700 7702 7717 7720 7722 7740 7743 7758 7785 |
| | 7795 7815 7819 7820 7830 7831 7833 7836 7838 7841 7848 7866 7868 7869 7871 7873 7874 7875 7876 7883 |
| | 7898 7899 7903 7904 7905 7906 7908 7910 7921 7925 7934 7937 7938 7941 7942 7946 7967 7974 7977 7980 |
| | 7984 7985 8011 8012 8016 8019 8024 8041 8044 8047 8049 8051 8056 8060 8061 8062 8070 8072 8077 8079 |
| | 8081 8083 8085 8104 8132 8166 8169 8172 8174 8175 8178 8185 8310 8365 8374 8375 8377 8381 8382 8400 |
| | 8402 8404 8407 8419 8423 8425 8426 8431 8436 8454 8455 8457 8458 8459 8463 8484 8486 8487 8489 8490 |
| | 8491 8492 8509 8513 8514 8516 8517 8542 8545 8546 8547 8549 8564 8565 8568 8570 8574 8576 8578 8580 |
| | 8583 8584 8588 8589 8591 8601 8603 8604 8606 8608 8609 8613 8616 8618 8622 8627 8634 8637 8640 8641 |
| | 8642 8644 8645 8647 8649 8653 8656 8657 8658 8659 8662 8663 8664 8667 8669 8672 8673 8678 8688 8691 |
| | 8693 8694 8700 8701 8705 8706 8709 8710 8713 8715 8718 8737 8739 8741 8742 8743 8746 8747 8748 8753 |
| | 8759 8764 8768 8776 8777 8779 8781 8782 8785 8786 8791 8796 8813 8815 8818 8819 8822 8837 8839 8841 |
| | 8842 8844 8847 8853 8855 8856 8857 8861 8865 8866 8869 8870 8871 8880 8881 8883 8884 8928 8931 8933 |
| | 8939 8949 8957 8960 8968 8970 8977 9003 9004 9005 9007 9020 9040 9041 9044 9045 9046 9047 9048 9049 |
| | 9051 9052 9072 9075 9076 9077 9079 9080 9082 9083 9086 9088 9090 9093 9108 9113 9114 9116 9117 9118 |
| | 9120 9121 9122 9124 9125 9127 9128 9130 9133 9154 9156 9160 9162 9164 9167 9168 9169 9172 9173 9174 |
| | 9176 9180 9184 9199 9200 9201 9202 9204 9206 9264 9272 9280 9297 9298 9301 9304 9319 9321 9325 9326 |
| | 9327 9329 9330 9335 9345 9349 9356 9357 9359 9360 9361 9362 9363 9364 9366 9371 9380 9382 9384 9385 |
| | 9387 9392 9393 9395 9399 9402 9404 9408 9410 9412 9413 9415 9417 9419 9422 9424 9425 9435 9437 9443 |
| | 9445 9451 9452 9454 9455 9456 9457 9460 9461 9465 9478 9479 9480 9481 9483 9484 9486 9487 9488 9492 |
| | 9493 9497 9499 9500 9503 9516 9522 9525 9526 9527 9529 9530 9531 9533 9534 9537 9538 9543 9547 9550 |
| | 9552 9553 9555 9557 9561 9563 9571 9574 9575 9576 9577 9578 9579 9580 9582 9583 9598 9599 9601 9602 |
| | 9603 9606 9607 9608 9611 9612 9614 9623 9624 9626 9627 9631 9633 9656 9660 9662 9668 9684 9707 9709 |
| | 9730 9732 9765 9768 9771 9772 9776 9788 9789 9790 9793 9796 9807 9808 9816 9818 9819 9821 9824 9845 |
| | 9846 9847 9849 9851 9853 9854 9862 9865 9868 9870 9872 9885 9888 9891 9892 9896 9898 9899 9900 9901 |
| | 9903 9904 9905 9906 9929 9930 9931 9932 9934 9935 9936 9939 9940 9941 9964 9966 9969 9970 9973 9975 |
| | 9977 9980 9982 9983 10002 10013 10015 10018 10021 10026 10038 10041 10044 10046 10048 10074 10077 |
| | 10104 10106 10108 10112 10122 10126 10130 10133 10136 10138 10139 10140 10143 10145 10148 10149 10152 |
| | 10154 10155 10158 10159 10161 10164 10166 10167 10168 10170 10172 10173 10175 10179 10181 10186 10188 |
| | 10192 10193 10194 10195 10196 10197 10199 10200 10205 10207 10209 10213 10214 10215 10216 10218 10220 |
| | 10221 10222 10224 10225 10226 10227 10228 10230 10232 10234 10236 10239 10240 10243 10244 10246 10248 |
| | 10249 10252 10253 10255 10256 10258 10260 10262 10263 10267 10272 10273 10274 10275 10280 10283 10287 |
| | 10289 10291 10295 10297 10301 10303 10308 10309 10310 10315 10316 10317 10318 10321 10323 10329 10331 |
| | 10332 10336 10339 10346 10347 10349 10363 10366 10369 10371 10372 10374 10375 10378 10384 10403 10406 |
| | 10407 10409 10410 10412 10413 10416 10417 10419 10420 10424 10427 10430 10433 10442 10444 10447 10449 |
| | 10452 10462 |
| 163: | 6919 6922 6926 6930 6932 6933 6946 6948 6950 6953 6955 6972 6994 7027 7028 7030 7033 7035 7044 |
| | 7047 7049 7050 7051 7054 7063 7067 7072 7073 7077 7080 7083 7085 7088 7100 7101 7106 7112 7116 7117 |
| | 7118 7124 7128 7129 7130 7132 7134 7136 7137 7138 7150 7153 7154 7156 7157 7175 7338 7368 7394 7396 |
| | 7398 7401 7403 7407 7412 7430 7431 7433 7438 7443 7445 7448 7454 7455 7467 7469 7472 7473 7478 7479 |
| | 7484 7493 7495 7499 7500 7501 7502 7514 7518 7519 7523 7524 7526 7527 7535 7539 7544 7546 7547 7549 |
| | 7550 7552 7553 7560 7562 7578 7585 7671 7680 7682 7692 7698 7700 7702 7717 7720 7722 7740 7743 7758 |
| | 7785 7795 7815 7819 7820 7830 7831 7833 7836 7838 7841 7848 7866 7868 7869 7871 7873 7874 7875 7876 |
| | 7883 7898 7899 7903 7904 7905 7906 7908 7910 7921 7925 7934 7937 7938 7941 7942 7946 7967 7974 7977 |
| | 7980 7984 7985 8011 8012 8016 8019 8024 8041 8044 8047 8049 8051 8056 8060 8061 8062 8070 8072 8077 |
| | 8079 8081 8083 8085 8104 8132 8166 8169 8172 8174 8175 8178 8185 8310 8365 8374 8375 8377 8381 8382 |
| | 8400 8402 8404 8407 8408 8419 8423 8425 8426 8431 8436 8454 8455 8457 8458 8459 8463 8484 8486 8487 |
| | 8489 8490 8491 8492 8509 8513 8514 8516 8517 8542 8545 8546 8547 8549 8564 8565 8568 8570 8574 8576 |
| | 8578 8580 8583 8584 8588 8589 8591 8601 8603 8604 8606 8608 8609 8613 8616 8618 8622 8627 8634 8637 |
| | 8640 8641 8642 8644 8645 8647 8649 8653 8656 8657 8658 8659 8662 8663 8664 8667 8669 8672 8673 8678 |
| | 8688 8691 8693 8694 8700 8701 8705 8706 8709 8710 8713 8715 8718 8737 8739 8741 8742 8743 8746 8747 |
| | 8748 8753 8759 8764 8768 8776 8777 8779 8781 8782 8785 8786 8791 8796 8813 8815 8818 8819 8822 8837 |
| | 8839 8841 8842 8844 8847 8853 8855 8856 8857 8861 8865 8866 8869 8870 8871 8880 8881 8883 8884 8928 |
| | 8931 8933 8939 8949 8957 8960 8968 8970 8977 9003 9004 9005 9007 9020 9040 9041 9044 9045 9046 9047 |
| | 9048 9049 9051 9052 9072 9075 9076 9077 9079 9080 9082 9083 9086 9088 9090 9093 9108 9113 9114 9116 |
| | 9117 9118 9120 9121 9122 9124 9125 9127 9128 9130 9133 9154 9156 9160 9162 9164 9167 9168 9169 9172 |
| | 9173 9174 9176 9180 9184 9199 9200 9201 9202 9204 9206 9264 9272 9280 9297 9298 9301 9304 9319 9321 |
| | 9325 9326 9327 9329 9330 9335 9345 9349 9356 9357 9359 9360 9361 9362 9363 9364 9366 9371 9380 9382 |
| | 9384 9385 9387 9392 9393 9395 9399 9402 9404 9408 9410 9412 9413 9415 9417 9419 9422 9424 9425 9435 |
| | 9437 9443 9445 9451 9452 9454 9455 9456 9457 9460 9461 9465 9478 9479 9480 9481 9483 9484 9486 9487 |
| | 9488 9492 9493 9497 9499 9500 9503 9516 9522 9525 9526 9527 9529 9530 9531 9533 9534 9537 9538 9543 |
| | 9547 9550 9552 9553 9555 9557 9561 9563 9571 9574 9575 9576 9577 9578 9579 9580 9582 9583 9598 9599 |
| | 9601 9602 9603 9606 9607 9608 9611 9612 9614 9623 9624 9626 9627 9631 9633 9656 9660 9662 9668 9684 |
| | 9707 9709 9730 9732 9765 9768 9771 9772 9776 9788 9789 9790 9793 9796 9807 9808 9816 9818 9819 9821 |
| | 9824 9845 9846 9847 9849 9851 9853 9854 9862 9865 9868 9870 9872 9885 9888 9891 9892 9896 9898 9899 |
| | 9900 9901 9903 9904 9905 9906 9929 9930 9931 9932 9934 9935 9936 9939 9940 9941 9964 9966 9969 9970 |
| | 9973 9975 9977 9980 9982 9983 10002 10013 10015 10018 10021 10026 10038 10041 10044 10046 10048 10074 |
| | 10077 10104 10106 10108 10112 10122 10126 10130 10133 10136 10138 10139 10140 10143 10145 10148 10149 |
| | 10152 10154 10155 10158 10159 10161 10164 10166 10167 10168 10170 10172 10173 10175 10179 10181 10186 |
| | 10188 10192 10193 10194 10195 10196 10197 10199 10200 10205 10207 10209 10213 10214 10215 10216 10218 |
| | 10220 10221 10222 10224 10225 10226 10227 10228 10230 10232 10234 10236 10239 10240 10243 10244 10246 |
| | 10248 10249 10252 10253 10255 10256 10258 10260 10262 10263 10267 10272 10273 10274 10275 10283 10287 |
| | 10289 10291 10295 10297 10301 10303 10308 10309 10310 10315 10316 10317 10318 10321 10323 10329 10331 |
| | 10332 10336 10339 10346 10347 10349 10363 10366 10369 10371 10372 10374 10375 10378 10384 10403 10406 |
| | 10407 10409 10410 10412 10413 10416 10417 10419 10420 10424 10427 10430 10433 10442 10444 10447 10449 |
| | 10452 10462 |
| 164: | 6919 6922 6926 6930 6932 6933 6946 6948 6950 6953 6955 6972 6994 7027 7028 7030 7033 7035 7044 |
| | 7047 7049 7050 7051 7054 7063 7067 7072 7073 7077 7080 7083 7085 7088 7100 7101 7106 7112 7116 7117 |
| | 7118 7124 7128 7129 7130 7132 7134 7136 7137 7138 7150 7153 7154 7156 7157 7175 7338 7368 7394 7396 |
| | 7398 7401 7403 7412 7430 7431 7433 7438 7443 7445 7448 7454 7455 7467 7469 7472 7473 7478 7479 7484 |
| | 7493 7495 7499 7500 7501 7502 7514 7518 7519 7523 7524 7526 7527 7535 7539 7544 7546 7547 7549 7550 |
| | 7552 7553 7560 7562 7578 7585 7671 7680 7682 7692 7698 7700 7702 7717 7720 7722 7740 7743 7758 7785 |
| | 7795 7815 7819 7820 7830 7831 7833 7836 7838 7841 7848 7866 7868 7869 7871 7873 7874 7875 7876 7883 |
| | 7898 7899 7903 7904 7905 7906 7908 7910 7921 7925 7934 7937 7938 7941 7942 7946 7967 7974 7977 7980 |
| | 7984 7985 8011 8012 8016 8019 8024 8041 8044 8047 8049 8051 8056 8060 8061 8062 8070 8072 8077 8079 |
| | 8081 8083 8085 8104 8132 8166 8169 8172 8174 8175 8178 8185 8310 8365 8374 8375 8377 8381 8382 8400 |
| | 8402 8404 8407 8408 8419 8423 8425 8426 8431 8436 8454 8455 8457 8458 8459 8463 8484 8486 8487 8489 |
| | 8490 8491 8492 8509 8513 8514 8516 8517 8542 8545 8546 8547 8549 8564 8565 8568 8570 8574 8576 8578 |
| | 8580 8583 8584 8588 8589 8591 8601 8603 8604 8606 8608 8609 8613 8616 8618 8622 8627 8634 8637 8640 |
| | 8641 8642 8644 8645 8647 8649 8653 8656 8657 8658 8659 8662 8663 8664 8667 8669 8672 8673 8678 8688 |
| | 8691 8693 8694 8700 8701 8705 8706 8709 8710 8713 8715 8718 8737 8739 8741 8742 8743 8746 8747 8748 |
| | 8753 8759 8764 8768 8776 8777 8779 8781 8782 8785 8786 8791 8796 8813 8815 8818 8819 8822 8837 8839 |
| | 8841 8842 8844 8847 8853 8855 8856 8857 8861 8865 8866 8869 8870 8871 8880 8881 8883 8884 8928 8931 |
| | 8933 8939 8949 8957 8960 8968 8970 8977 9003 9004 9005 9007 9020 9040 9041 9044 9045 9046 9047 9048 |
| | 9049 9051 9052 9072 9075 9076 9077 9079 9080 9082 9083 9086 9088 9090 9093 9108 9113 9114 9116 9117 |
| | 9118 9120 9121 9122 9124 9125 9127 9128 9130 9133 9154 9156 9160 9162 9164 9167 9168 9169 9172 9173 |
| | 9174 9176 9180 9184 9199 9200 9201 9202 9204 9206 9264 9272 9280 9297 9298 9301 9304 9319 9321 9325 |
| | 9326 9327 9329 9330 9335 9345 9349 9356 9357 9359 9360 9361 9362 9363 9364 9366 9371 9380 9382 9384 |
| | 9385 9387 9392 9393 9395 9399 9400 9402 9404 9408 9410 9412 9413 9415 9417 9419 9422 9424 9425 9435 |
| | 9437 9443 9445 9451 9452 9454 9455 9456 9457 9460 9461 9465 9478 9479 9480 9481 9483 9484 9486 9487 |
| | 9488 9492 9493 9497 9499 9500 9503 9516 9522 9525 9526 9527 9529 9530 9531 9533 9534 9537 9538 9543 |
| | 9547 9550 9552 9553 9555 9557 9561 9563 9571 9574 9575 9576 9577 9578 9579 9580 9582 9583 9598 9599 |
| | 9601 9602 9603 9606 9607 9608 9611 9612 9614 9623 9624 9626 9627 9631 9633 9656 9660 9662 9668 9684 |
| | 9707 9709 9730 9732 9765 9768 9771 9772 9776 9788 9789 9790 9793 9796 9807 9808 9816 9818 9819 9821 |
| | 9824 9845 9846 9847 9849 9851 9853 9854 9862 9865 9868 9870 9872 9885 9888 9891 9892 9896 9898 9899 |
| | 9900 9901 9903 9904 9905 9906 9929 9930 9931 9932 9934 9935 9936 9939 9940 9941 9964 9966 9969 9970 |
| | 9973 9975 9977 9980 9982 9983 10002 10013 10015 10018 10021 10026 10038 10041 10044 10046 10048 10074 |
| | 10077 10104 10106 10108 10112 10122 10126 10130 10133 10136 10138 10139 10140 10143 10145 10148 10149 |
| | 10152 10154 10155 10158 10159 10161 10164 10166 10167 10168 10170 10172 10173 10175 10179 10181 10186 |
| | 10188 10192 10193 10194 10195 10196 10197 10199 10200 10205 10207 10209 10213 10214 10215 10216 10218 |
| | 10220 10221 10222 10224 10225 10226 10227 10228 10230 10232 10234 10236 10239 10240 10243 10244 10246 |
| | 10248 10249 10252 10253 10255 10256 10258 10260 10262 10263 10267 10272 10273 10274 10275 10283 10287 |
| | 10289 10291 10295 10297 10301 10303 10308 10309 10310 10315 10316 10317 10318 10321 10323 10329 10331 |
| | 10332 10336 10339 10346 10347 10349 10363 10366 10369 10371 10372 10374 10375 10378 10384 10403 10406 |
| | 10407 10409 10410 10412 10413 10416 10417 10419 10420 10424 10427 10430 10433 10442 10444 10447 10449 |
| | 10452 10462 |
| 165: | 6919 6922 6926 6930 6932 6933 6946 6948 6950 6953 6955 6957 6971 6972 6986 6991 6994 6998 7011 |
| | 7027 7028 7030 7033 7035 7044 7047 7049 7050 7051 7054 7056 7063 7067 7072 7077 7080 7083 7085 7088 |
| | 7093 7100 7101 7106 7112 7116 7117 7118 7124 7128 7129 7130 7132 7134 7136 7137 7138 7150 7153 7154 |
| | 7156 7157 7162 7164 7175 7216 7269 7327 7386 7394 7396 7398 7401 7403 7407 7412 7417 7430 7431 7433 |
| | 7438 7443 7445 7448 7455 7467 7469 7470 7472 7473 7478 7479 7485 7490 7493 7494 7495 7499 7500 7501 |
| | 7502 7511 7514 7518 7519 7523 7524 7526 7527 7539 7541 7544 7546 7547 7549 7550 7552 7560 7562 7566 |
| | 7578 7585 7756 7761 7762 7781 7786 7792 7804 7817 7830 7831 7833 7834 7836 7838 7840 7841 7849 7866 |
| | 7868 7869 7871 7873 7875 7879 7881 7884 7898 7899 7903 7904 7905 7906 7908 7910 7914 7921 7925 7934 |
| | 7937 7938 7941 7942 7943 7944 7946 7951 7965 7967 7974 7977 7980 7984 7985 8005 8011 8012 8016 8019 |
| | 8024 8041 8044 8047 8049 8051 8064 8066 8072 8077 8079 8081 8083 8085 8147 8169 8170 8172 8174 8175 |
| | 8178 8224 8233 8310 8328 8365 8374 8375 8377 8381 8382 8385 8386 8400 8401 8402 8404 8407 8408 8423 |
| | 8425 8426 8431 8436 8454 8455 8458 8459 8463 8472 8476 8487 8489 8490 8491 8492 8509 8513 8514 8516 |
| | 8517 8531 8542 8545 8546 8547 8549 8551 8552 8555 8564 8565 8568 8570 8574 8575 8576 8578 8579 8580 |
| | 8588 8591 8601 8603 8604 8606 8609 8613 8616 8617 8618 8622 8627 8640 8641 8642 8644 8645 8647 8649 |
| | 8653 8654 8656 8657 8659 8662 8663 8664 8667 8669 8672 8673 8678 8688 8689 8691 8693 8700 8705 8706 |
| | 8709 8710 8713 8715 8718 8729 8739 8741 8743 8746 8747 8748 8753 8759 8764 8776 8777 8779 8781 8782 |
| | 8785 8786 8791 8796 8813 8815 8818 8819 8822 8832 8833 8839 8841 8842 8844 8847 8848 8853 8855 8856 |
| | 8857 8859 8866 8869 8871 8877 8880 8881 8883 8884 8892 8898 8924 8933 8939 8953 8954 8955 8957 8958 |
| | 8960 8968 8970 8977 9003 9004 9005 9006 9007 9040 9041 9044 9045 9046 9047 9048 9049 9051 9052 9072 |
| | 9075 9076 9077 9079 9080 9082 9083 9086 9088 9090 9093 9108 9113 9114 9116 9117 9118 9120 9121 9122 |
| | 9124 9125 9127 9128 9130 9133 9154 9156 9160 9162 9164 9167 9168 9169 9172 9173 9174 9176 9180 9184 |
| | 9199 9200 9201 9202 9204 9206 9249 9264 9270 9298 9299 9301 9304 9315 9319 9320 9321 9325 9326 9327 |
| | 9329 9330 9331 9335 9338 9343 9344 9345 9349 9355 9356 9357 9358 9359 9360 9362 9363 9364 9366 9371 |
| | 9377 9380 9382 9384 9385 9387 9392 9393 9395 9396 9399 9400 9404 9410 9415 9417 9419 9422 9424 9425 |
| | 9430 9437 9443 9445 9451 9452 9454 9455 9456 9457 9458 9460 9461 9470 9471 9478 9480 9481 9483 9484 |
| | 9486 9487 9488 9492 9493 9495 9497 9499 9500 9503 9516 9522 9525 9526 9527 9529 9530 9531 9533 9534 |
| | 9537 9543 9547 9550 9552 9553 9555 9557 9561 9562 9563 9571 9574 9575 9576 9577 9578 9579 9580 9583 |
| | 9589 9590 9593 9597 9598 9599 9600 9601 9602 9603 9607 9608 9611 9612 9614 9620 9623 9624 9626 9627 |
| | 9631 9633 9638 9667 9669 9670 9686 9688 9712 9722 9724 9730 9732 9744 9748 9755 9758 9763 9765 9768 |
| | 9771 9772 9776 9781 9782 9784 9785 9788 9789 9790 9793 9796 9799 9805 9806 9808 9809 9812 9813 9814 |
| | 9816 9818 9819 9821 9824 9829 9837 9841 9845 9846 9847 9849 9851 9853 9854 9862 9865 9868 9870 9872 |
| | 9885 9888 9891 9892 9896 9898 9899 9900 9901 9903 9904 9905 9906 9926 9929 9930 9931 9932 9934 9935 |
| | 9936 9939 9940 9941 9963 9964 9966 9969 9970 9973 9975 9977 9980 9982 9983 10002 10003 10006 10013 |
| | 10014 10015 10018 10021 10026 10038 10041 10044 10046 10048 10058 10074 10108 10111 10122 10133 10136 |
| | 10138 10139 10140 10143 10145 10149 10150 10152 10154 10158 10159 10168 10170 10172 10173 10175 10179 |
| | 10181 10186 10188 10192 10193 10194 10195 10196 10197 10200 10205 10207 10209 10213 10214 10215 10216 |
| | 10218 10219 10220 10221 10222 10224 10226 10227 10228 10230 10234 10236 10239 10240 10243 10244 10246 |
| | 10248 10249 10251 10252 10253 10255 10256 10258 10260 10262 10263 10267 10271 10273 10274 10275 10279 |
| | 10283 10287 10289 10291 10295 10297 10300 10301 10303 10308 10309 10310 10315 10316 10317 10318 10321 |
| | 10329 10331 10332 10336 10339 10345 10346 10347 10349 10355 10357 10362 10363 10366 10369 10371 10372 |
| | 10374 10375 10378 10384 10391 10393 10403 10406 10407 10409 10410 10412 10413 10416 10417 10419 10424 |
| | 10427 10430 10433 10442 10444 10447 10449 10452 10462 |
| 166: | 6919 6922 6926 6930 6932 6933 6946 6948 6950 6953 6955 6957 6971 6972 6986 6991 6994 6998 7011 |
| | 7027 7028 7030 7033 7035 7044 7047 7049 7050 7051 7054 7056 7063 7067 7072 7077 7080 7083 7085 7088 |
| | 7093 7100 7101 7106 7112 7116 7117 7118 7124 7128 7129 7130 7132 7134 7136 7137 7138 7150 7153 7154 |
| | 7156 7157 7162 7164 7175 7216 7269 7327 7386 7394 7396 7398 7401 7403 7407 7412 7417 7430 7431 7433 |
| | 7438 7443 7445 7448 7455 7467 7469 7470 7472 7473 7478 7479 7485 7490 7493 7494 7495 7497 7499 7500 |
| | 7501 7502 7514 7518 7519 7523 7524 7526 7527 7539 7541 7544 7546 7547 7549 7550 7552 7560 7562 7566 |
| | 7578 7585 7756 7761 7762 7781 7786 7792 7804 7817 7830 7831 7833 7834 7836 7838 7840 7841 7849 7866 |
| | 7868 7869 7871 7873 7875 7879 7881 7884 7898 7899 7903 7904 7905 7906 7908 7910 7914 7921 7925 7934 |
| | 7937 7938 7941 7942 7943 7944 7946 7951 7965 7967 7974 7977 7980 7984 7985 8005 8011 8012 8016 8019 |
| | 8024 8041 8044 8047 8049 8051 8064 8066 8072 8077 8079 8081 8083 8085 8147 8169 8170 8172 8174 8175 |
| | 8178 8224 8233 8310 8328 8365 8374 8375 8377 8381 8382 8385 8386 8400 8401 8402 8404 8407 8408 8423 |
| | 8425 8426 8431 8436 8454 8455 8458 8459 8463 8472 8476 8487 8489 8490 8491 8492 8509 8513 8514 8516 |
| | 8517 8531 8542 8545 8546 8547 8549 8551 8552 8555 8564 8565 8568 8570 8574 8575 8576 8578 8579 8580 |
| | 8588 8591 8601 8603 8604 8606 8609 8613 8616 8617 8618 8622 8627 8640 8641 8642 8644 8645 8647 8649 |
| | 8653 8654 8656 8657 8659 8662 8663 8664 8667 8669 8672 8673 8678 8688 8689 8691 8693 8700 8705 8706 |
| | 8709 8710 8713 8715 8718 8729 8739 8741 8743 8746 8747 8748 8753 8759 8764 8776 8777 8779 8781 8782 |
| | 8785 8786 8791 8796 8813 8815 8818 8819 8822 8832 8833 8839 8841 8842 8844 8847 8848 8853 8855 8856 |
| | 8857 8859 8866 8869 8871 8877 8880 8881 8883 8884 8892 8898 8924 8933 8939 8953 8954 8955 8957 8958 |
| | 8960 8968 8970 8977 9003 9004 9005 9006 9007 9040 9041 9044 9045 9046 9047 9048 9049 9051 9052 9072 |
| | 9075 9076 9077 9079 9080 9082 9083 9086 9088 9090 9093 9108 9113 9114 9116 9117 9118 9120 9121 9122 |
| | 9124 9125 9127 9128 9130 9133 9154 9156 9160 9162 9164 9167 9168 9169 9172 9173 9174 9176 9180 9184 |
| | 9199 9200 9201 9202 9204 9206 9249 9264 9270 9298 9299 9301 9304 9315 9319 9320 9321 9325 9326 9327 |
| | 9329 9330 9331 9335 9338 9343 9344 9345 9349 9355 9356 9357 9358 9359 9360 9362 9363 9364 9366 9371 |
| | 9377 9380 9382 9384 9385 9387 9392 9393 9395 9396 9399 9400 9404 9410 9415 9417 9419 9422 9424 9425 |
| | 9430 9437 9443 9445 9451 9452 9454 9455 9456 9457 9458 9460 9461 9470 9471 9478 9480 9481 9483 9484 |
| | 9486 9487 9488 9492 9493 9495 9497 9499 9500 9503 9516 9522 9525 9526 9527 9529 9530 9531 9533 9534 |
| | 9537 9543 9547 9550 9552 9553 9555 9557 9561 9562 9563 9571 9574 9575 9576 9577 9578 9579 9580 9583 |
| | 9589 9590 9593 9597 9598 9599 9600 9601 9602 9603 9607 9608 9611 9612 9614 9620 9623 9624 9626 9627 |
| | 9631 9633 9638 9667 9669 9670 9686 9688 9712 9722 9724 9730 9732 9744 9748 9755 9758 9763 9765 9768 |
| | 9771 9772 9776 9781 9782 9784 9785 9788 9789 9790 9793 9796 9799 9805 9806 9808 9809 9812 9813 9814 |
| | 9816 9818 9819 9821 9824 9829 9837 9841 9845 9846 9847 9849 9851 9853 9854 9862 9865 9868 9870 9872 |
| | 9885 9888 9891 9892 9896 9898 9899 9900 9901 9903 9904 9905 9906 9926 9929 9930 9931 9932 9934 9935 |
| | 9936 9939 9940 9941 9963 9964 9966 9969 9970 9973 9975 9977 9980 9982 9983 10002 10003 10006 10013 |
| | 10014 10015 10018 10021 10026 10038 10041 10044 10046 10048 10058 10074 10108 10111 10122 10133 10136 |
| | 10138 10139 10140 10143 10145 10149 10150 10152 10154 10158 10159 10168 10170 10172 10173 10175 10179 |
| | 10181 10186 10188 10192 10193 10194 10195 10196 10197 10200 10205 10207 10209 10213 10214 10215 10216 |
| | 10218 10219 10220 10221 10222 10224 10226 10227 10228 10230 10234 10236 10239 10240 10243 10244 10246 |
| | 10248 10249 10251 10252 10253 10255 10256 10258 10260 10262 10263 10267 10271 10273 10274 10275 10279 |
| | 10283 10287 10289 10291 10295 10297 10300 10301 10303 10308 10309 10310 10315 10316 10317 10318 10321 |
| | 10329 10331 10332 10336 10339 10345 10346 10347 10349 10355 10357 10362 10363 10366 10369 10371 10372 |
| | 10374 10375 10378 10384 10391 10393 10403 10406 10407 10409 10410 10412 10413 10416 10417 10419 10424 |
| | 10427 10430 10433 10442 10444 10447 10449 10452 10462 |
| 167: | 6919 6922 6926 6930 6932 6933 6946 6948 6950 6953 6955 6957 6971 6972 6986 6991 6994 6998 7011 |
| | 7027 7028 7030 7033 7035 7044 7047 7049 7050 7051 7054 7056 7063 7067 7072 7077 7080 7083 7085 7088 |
| | 7093 7100 7101 7106 7112 7116 7117 7118 7124 7128 7129 7130 7132 7134 7136 7137 7138 7150 7153 7154 |
| | 7156 7157 7162 7164 7175 7216 7269 7327 7386 7394 7396 7398 7401 7403 7407 7412 7417 7430 7431 7433 |
| | 7438 7443 7445 7448 7455 7467 7469 7470 7472 7473 7478 7479 7485 7490 7493 7494 7495 7497 7499 7500 |
| | 7501 7502 7514 7518 7519 7523 7524 7526 7527 7539 7541 7544 7546 7547 7549 7550 7552 7560 7562 7566 |
| | 7578 7585 7756 7761 7762 7781 7786 7792 7804 7817 7830 7831 7833 7834 7836 7838 7840 7841 7849 7866 |
| | 7868 7869 7871 7873 7875 7879 7881 7884 7898 7899 7903 7904 7905 7906 7908 7910 7914 7921 7925 7934 |
| | 7937 7938 7941 7942 7943 7944 7946 7951 7965 7967 7974 7977 7980 7984 7985 8005 8011 8012 8016 8019 |
| | 8024 8041 8044 8047 8049 8051 8064 8066 8072 8077 8079 8081 8083 8085 8147 8169 8170 8172 8174 8175 |
| | 8178 8224 8233 8310 8328 8365 8374 8375 8377 8381 8382 8385 8386 8400 8401 8402 8404 8407 8408 8423 |
| | 8425 8426 8431 8436 8454 8455 8458 8459 8463 8472 8476 8487 8489 8490 8491 8492 8509 8513 8514 8516 |
| | 8517 8531 8542 8545 8546 8547 8549 8551 8552 8555 8564 8565 8568 8570 8574 8575 8576 8578 8579 8580 |
| | 8588 8591 8601 8603 8604 8606 8609 8613 8616 8617 8618 8622 8627 8640 8641 8642 8644 8645 8647 8649 |
| | 8653 8654 8656 8657 8659 8662 8663 8664 8667 8669 8672 8673 8678 8688 8689 8691 8693 8700 8705 8706 |
| | 8709 8710 8713 8715 8718 8729 8739 8741 8743 8746 8747 8748 8753 8759 8764 8776 8777 8779 8781 8782 |
| | 8785 8786 8791 8796 8813 8815 8818 8819 8822 8832 8833 8839 8841 8842 8844 8847 8848 8853 8855 8856 |
| | 8857 8859 8866 8869 8871 8877 8880 8881 8883 8884 8892 8898 8924 8933 8939 8953 8954 8955 8957 8958 |
| | 8960 8968 8970 8977 9003 9004 9005 9006 9007 9040 9041 9044 9045 9046 9047 9048 9049 9051 9052 9072 |
| | 9075 9076 9077 9079 9080 9082 9083 9086 9088 9090 9093 9108 9113 9114 9116 9117 9118 9120 9121 9122 |
| | 9124 9125 9127 9128 9130 9133 9154 9156 9160 9162 9164 9167 9168 9169 9172 9173 9174 9176 9180 9184 |
| | 9199 9200 9201 9202 9204 9206 9249 9264 9270 9298 9299 9301 9304 9315 9319 9320 9321 9325 9326 9327 |
| | 9329 9330 9331 9335 9338 9343 9344 9345 9349 9355 9356 9357 9358 9359 9360 9362 9363 9364 9366 9371 |
| | 9377 9380 9382 9384 9385 9387 9392 9393 9395 9396 9399 9400 9404 9410 9415 9417 9419 9422 9424 9425 |
| | 9430 9437 9443 9445 9451 9452 9454 9455 9456 9457 9458 9460 9461 9470 9471 9478 9480 9481 9483 9484 |
| | 9486 9487 9488 9492 9493 9495 9497 9499 9500 9503 9516 9522 9525 9526 9527 9529 9530 9531 9533 9534 |
| | 9537 9543 9547 9550 9552 9553 9555 9557 9561 9562 9563 9571 9574 9575 9576 9577 9578 9579 9580 9583 |
| | 9589 9590 9593 9597 9598 9599 9600 9601 9602 9603 9607 9608 9611 9612 9614 9620 9623 9624 9626 9627 |
| | 9631 9633 9638 9667 9669 9670 9686 9688 9712 9722 9724 9730 9732 9744 9748 9755 9758 9763 9765 9768 |
| | 9771 9772 9776 9781 9782 9784 9785 9788 9789 9790 9793 9796 9799 9805 9806 9808 9809 9812 9813 9814 |
| | 9816 9818 9819 9821 9824 9829 9837 9841 9845 9846 9847 9849 9851 9853 9854 9862 9865 9868 9870 9872 |
| | 9885 9888 9891 9892 9896 9898 9899 9900 9901 9903 9904 9905 9906 9926 9929 9930 9931 9932 9934 9935 |
| | 9936 9939 9940 9941 9963 9964 9966 9969 9970 9973 9975 9977 9980 9982 9983 10002 10003 10006 10013 |
| | 10014 10015 10018 10021 10026 10038 10041 10044 10046 10048 10058 10074 10108 10111 10122 10133 10136 |
| | 10138 10139 10140 10143 10145 10149 10150 10152 10154 10158 10159 10168 10170 10172 10173 10175 10179 |
| | 10181 10186 10188 10192 10193 10194 10195 10196 10197 10200 10205 10207 10209 10213 10214 10215 10216 |
| | 10218 10219 10220 10221 10222 10224 10226 10227 10228 10230 10234 10236 10239 10240 10243 10244 10246 |
| | 10248 10249 10251 10252 10253 10255 10256 10258 10260 10262 10263 10267 10271 10273 10274 10275 10279 |
| | 10283 10287 10289 10291 10295 10297 10300 10301 10303 10308 10309 10310 10315 10316 10317 10318 10321 |
| | 10329 10331 10332 10336 10339 10345 10346 10347 10349 10355 10357 10362 10363 10366 10369 10371 10372 |
| | 10374 10375 10378 10384 10391 10393 10403 10406 10407 10409 10410 10412 10413 10416 10417 10419 10424 |
| | 10427 10430 10433 10442 10444 10447 10449 10452 10462 |
| 168: | 6919 6922 6926 6930 6932 6933 6946 6948 6950 6953 6955 6972 6994 7027 7028 7030 7033 7035 7044 |
| | 7047 7049 7050 7051 7054 7063 7067 7072 7073 7077 7080 7083 7085 7088 7100 7101 7106 7112 7116 7117 |
| | 7118 7124 7128 7129 7130 7132 7134 7136 7137 7138 7150 7153 7154 7156 7157 7175 7338 7368 7394 7396 |
| | 7398 7401 7403 7412 7430 7431 7433 7438 7443 7445 7448 7454 7455 7467 7469 7472 7473 7478 7479 7484 |
| | 7493 7495 7499 7500 7501 7502 7514 7518 7519 7523 7524 7526 7527 7535 7539 7544 7546 7547 7549 7550 |
| | 7552 7553 7560 7562 7578 7585 7671 7680 7682 7692 7698 7700 7702 7717 7720 7722 7740 7743 7758 7785 |
| | 7795 7815 7819 7820 7830 7831 7833 7836 7838 7841 7848 7866 7868 7869 7871 7873 7874 7875 7876 7883 |
| | 7898 7899 7903 7904 7905 7906 7908 7910 7921 7925 7934 7937 7938 7941 7942 7946 7967 7974 7977 7980 |
| | 7984 7985 8011 8012 8016 8019 8024 8041 8044 8047 8049 8051 8056 8060 8061 8062 8070 8072 8077 8079 |
| | 8081 8083 8085 8104 8132 8166 8169 8172 8174 8175 8178 8185 8310 8365 8374 8375 8377 8381 8382 8400 |
| | 8402 8404 8407 8419 8423 8425 8426 8431 8436 8454 8455 8457 8458 8459 8463 8484 8486 8487 8489 8490 |
| | 8491 8492 8509 8513 8514 8516 8517 8542 8545 8546 8547 8549 8564 8565 8568 8570 8574 8576 8578 8580 |
| | 8583 8584 8588 8589 8591 8601 8603 8604 8606 8608 8609 8613 8616 8618 8622 8627 8634 8637 8640 8641 |
| | 8642 8644 8645 8647 8649 8653 8656 8657 8658 8659 8662 8663 8664 8667 8669 8672 8673 8678 8688 8691 |
| | 8693 8694 8700 8701 8705 8706 8709 8710 8713 8715 8718 8737 8739 8741 8742 8743 8746 8747 8748 8753 |
| | 8759 8764 8768 8776 8777 8779 8781 8782 8785 8786 8791 8796 8813 8815 8818 8819 8822 8837 8839 8841 |
| | 8842 8844 8847 8853 8855 8856 8857 8861 8865 8866 8869 8870 8871 8880 8881 8883 8884 8928 8931 8933 |
| | 8939 8949 8957 8960 8968 8970 8977 9003 9004 9005 9007 9020 9040 9041 9044 9045 9046 9047 9048 9049 |
| | 9051 9052 9072 9075 9076 9077 9079 9080 9082 9083 9086 9088 9090 9093 9108 9113 9114 9116 9117 9118 |
| | 9120 9121 9122 9124 9125 9127 9128 9130 9133 9154 9156 9160 9162 9164 9167 9168 9169 9172 9173 9174 |
| | 9176 9180 9184 9199 9200 9201 9202 9204 9206 9264 9272 9280 9297 9298 9301 9304 9319 9321 9325 9326 |
| | 9327 9329 9330 9335 9345 9349 9356 9357 9359 9360 9361 9362 9363 9364 9366 9371 9380 9382 9384 9385 |
| | 9387 9392 9393 9395 9399 9400 9402 9404 9408 9410 9412 9413 9415 9417 9419 9422 9424 9425 9435 9437 |
| | 9443 9445 9451 9452 9454 9455 9456 9457 9460 9461 9465 9478 9479 9480 9481 9483 9484 9486 9487 9488 |
| | 9492 9493 9497 9499 9500 9503 9516 9522 9525 9526 9527 9529 9530 9531 9533 9534 9537 9538 9543 9547 |
| | 9550 9552 9553 9555 9557 9561 9563 9571 9574 9575 9576 9577 9578 9579 9580 9582 9583 9598 9599 9601 |
| | 9602 9603 9606 9607 9608 9611 9612 9614 9623 9624 9626 9627 9631 9633 9656 9660 9662 9668 9684 9707 |
| | 9709 9730 9732 9765 9768 9771 9772 9776 9788 9789 9790 9793 9796 9807 9808 9816 9818 9819 9821 9824 |
| | 9845 9846 9847 9849 9851 9853 9854 9862 9865 9868 9870 9872 9885 9888 9891 9892 9896 9898 9899 9900 |
| | 9901 9903 9904 9905 9906 9929 9930 9931 9932 9934 9935 9936 9939 9940 9941 9964 9966 9969 9970 9973 |
| | 9975 9977 9980 9982 9983 10002 10013 10015 10018 10021 10026 10038 10041 10044 10046 10048 10074 |
| | 10077 10104 10106 10108 10112 10122 10126 10130 10133 10136 10138 10139 10140 10143 10145 10148 10149 |
| | 10152 10154 10155 10158 10159 10161 10164 10166 10167 10168 10170 10172 10173 10175 10179 10181 10186 |
| | 10188 10192 10193 10194 10195 10196 10197 10199 10200 10205 10207 10209 10213 10214 10215 10216 10218 |
| | 10220 10221 10222 10224 10225 10226 10227 10228 10230 10232 10234 10236 10239 10240 10243 10244 10246 |
| | 10248 10249 10252 10253 10255 10256 10258 10260 10262 10263 10267 10272 10273 10274 10275 10280 10283 |
| | 10287 10289 10291 10295 10297 10301 10303 10308 10309 10310 10315 10316 10317 10318 10321 10323 10329 |
| | 10331 10332 10336 10339 10346 10347 10349 10363 10366 10369 10371 10372 10374 10375 10378 10384 10403 |
| | 10406 10407 10409 10410 10412 10413 10416 10417 10419 10420 10424 10427 10430 10433 10442 10444 10447 |
| | 10449 10452 10462 |
| 169: | 6919 6922 6926 6930 6932 6933 6946 6948 6950 6953 6955 6957 6971 6972 6986 6991 6994 6998 7011 |
| | 7027 7028 7030 7033 7035 7044 7047 7049 7050 7051 7054 7056 7063 7067 7072 7077 7080 7083 7085 7088 |
| | 7093 7100 7101 7106 7112 7116 7117 7118 7124 7128 7129 7130 7132 7134 7136 7137 7138 7150 7153 7154 |
| | 7156 7157 7162 7164 7175 7216 7269 7327 7386 7394 7396 7398 7401 7403 7407 7412 7417 7430 7431 7433 |
| | 7438 7443 7445 7448 7455 7467 7469 7470 7472 7473 7478 7479 7485 7490 7493 7494 7495 7497 7499 7500 |
| | 7501 7502 7514 7518 7519 7523 7524 7526 7527 7539 7541 7544 7546 7547 7549 7550 7552 7560 7562 7566 |
| | 7578 7585 7756 7761 7762 7781 7786 7792 7804 7817 7830 7831 7833 7834 7836 7838 7840 7841 7849 7866 |
| | 7868 7869 7871 7873 7875 7879 7881 7884 7898 7899 7903 7904 7905 7906 7908 7910 7914 7921 7925 7934 |
| | 7937 7938 7941 7942 7943 7944 7946 7951 7965 7967 7974 7977 7980 7984 7985 8005 8011 8012 8016 8019 |
| | 8024 8041 8044 8047 8049 8051 8064 8066 8072 8077 8079 8081 8083 8085 8147 8169 8170 8172 8174 8175 |
| | 8178 8224 8233 8310 8328 8365 8374 8375 8377 8381 8382 8385 8386 8400 8401 8402 8404 8407 8408 8423 |
| | 8425 8426 8431 8436 8454 8455 8458 8459 8463 8472 8476 8487 8489 8490 8491 8492 8509 8513 8514 8516 |
| | 8517 8531 8542 8545 8546 8547 8549 8551 8552 8555 8564 8565 8568 8570 8574 8575 8576 8578 8579 8580 |
| | 8588 8591 8601 8603 8604 8606 8609 8613 8616 8617 8618 8622 8627 8640 8641 8642 8644 8645 8647 8649 |
| | 8653 8654 8656 8657 8659 8662 8663 8664 8667 8669 8672 8673 8678 8688 8689 8691 8693 8700 8705 8706 |
| | 8709 8710 8713 8715 8718 8729 8739 8741 8743 8746 8747 8748 8753 8759 8764 8776 8777 8779 8781 8782 |
| | 8785 8786 8791 8796 8813 8815 8818 8819 8822 8832 8833 8839 8841 8842 8844 8847 8848 8853 8855 8856 |
| | 8857 8859 8866 8869 8871 8877 8880 8881 8883 8884 8892 8898 8924 8933 8939 8953 8954 8955 8957 8958 |
| | 8960 8968 8970 8977 9003 9004 9005 9006 9007 9040 9041 9044 9045 9046 9047 9048 9049 9051 9052 9072 |
| | 9075 9076 9077 9079 9080 9082 9083 9086 9088 9090 9093 9108 9113 9114 9116 9117 9118 9120 9121 9122 |
| | 9124 9125 9127 9128 9130 9133 9154 9156 9160 9162 9164 9167 9168 9169 9172 9173 9174 9176 9180 9184 |
| | 9199 9200 9201 9202 9204 9206 9249 9264 9270 9298 9299 9301 9304 9315 9319 9320 9321 9325 9326 9327 |
| | 9329 9330 9331 9335 9338 9343 9344 9345 9349 9355 9356 9357 9358 9359 9360 9362 9363 9364 9366 9371 |
| | 9377 9380 9382 9384 9385 9387 9392 9393 9395 9396 9399 9400 9404 9410 9415 9417 9419 9422 9424 9425 |
| | 9430 9437 9443 9445 9451 9452 9454 9455 9456 9457 9458 9460 9461 9470 9471 9478 9480 9481 9483 9484 |
| | 9486 9487 9488 9492 9493 9495 9497 9499 9500 9503 9516 9522 9525 9526 9527 9529 9530 9531 9533 9534 |
| | 9537 9543 9547 9550 9552 9553 9555 9557 9561 9562 9563 9571 9574 9575 9576 9577 9578 9579 9580 9583 |
| | 9589 9590 9593 9597 9598 9599 9600 9601 9602 9603 9607 9608 9611 9612 9614 9620 9623 9624 9626 9627 |
| | 9631 9633 9638 9667 9669 9670 9686 9688 9712 9722 9724 9730 9732 9744 9748 9755 9758 9763 9765 9768 |
| | 9771 9772 9776 9781 9782 9784 9785 9788 9789 9790 9793 9796 9799 9805 9806 9808 9809 9812 9813 9814 |
| | 9816 9818 9819 9821 9824 9829 9837 9841 9845 9846 9847 9849 9851 9853 9854 9862 9865 9868 9870 9872 |
| | 9885 9888 9891 9892 9896 9898 9899 9900 9901 9903 9904 9905 9906 9926 9929 9930 9931 9932 9934 9935 |
| | 9936 9939 9940 9941 9963 9964 9966 9969 9970 9973 9975 9977 9980 9982 9983 10002 10003 10006 10013 |
| | 10014 10015 10018 10021 10026 10038 10041 10044 10046 10048 10058 10074 10108 10111 10122 10133 10136 |
| | 10138 10139 10140 10143 10145 10149 10150 10152 10154 10158 10159 10168 10170 10172 10173 10175 10179 |
| | 10181 10186 10188 10192 10193 10194 10195 10196 10197 10200 10205 10207 10209 10213 10214 10215 10216 |
| | 10218 10219 10220 10221 10222 10224 10226 10227 10228 10230 10234 10236 10239 10240 10243 10244 10246 |
| | 10248 10249 10251 10252 10253 10255 10256 10258 10260 10262 10263 10267 10271 10273 10274 10275 10279 |
| | 10283 10287 10289 10291 10295 10297 10300 10301 10303 10308 10309 10310 10315 10316 10317 10318 10321 |
| | 10329 10331 10332 10336 10339 10345 10346 10347 10349 10355 10357 10362 10363 10366 10369 10371 10372 |
| | 10374 10375 10378 10384 10391 10393 10403 10406 10407 10409 10410 10412 10413 10416 10417 10419 10424 |
| | 10427 10430 10433 10442 10444 10447 10449 10452 10462 |
| 170: | 6919 6922 6926 6930 6932 6933 6946 6948 6950 6953 6955 6957 6971 6972 6986 6991 6994 6998 7011 |
| | 7027 7028 7030 7033 7035 7044 7047 7049 7050 7051 7054 7056 7063 7067 7072 7077 7080 7083 7085 7088 |
| | 7093 7100 7101 7106 7112 7116 7117 7118 7124 7128 7129 7130 7132 7134 7136 7137 7138 7150 7153 7154 |
| | 7156 7157 7162 7164 7175 7216 7269 7327 7386 7394 7396 7398 7401 7403 7407 7412 7417 7430 7431 7433 |
| | 7438 7443 7445 7448 7455 7467 7469 7470 7472 7473 7478 7479 7485 7490 7493 7494 7495 7497 7499 7500 |
| | 7501 7502 7514 7518 7519 7523 7524 7526 7527 7539 7541 7544 7546 7547 7549 7550 7552 7560 7562 7566 |
| | 7578 7585 7756 7761 7762 7781 7786 7792 7804 7817 7830 7831 7833 7834 7836 7838 7840 7841 7849 7866 |
| | 7868 7869 7871 7873 7875 7879 7881 7884 7898 7899 7903 7904 7905 7906 7908 7910 7914 7921 7925 7934 |
| | 7937 7938 7941 7942 7943 7944 7946 7951 7965 7967 7974 7977 7980 7984 7985 8005 8011 8012 8016 8019 |
| | 8024 8041 8044 8047 8049 8051 8064 8066 8072 8077 8079 8081 8083 8085 8147 8169 8170 8172 8174 8175 |
| | 8178 8224 8233 8310 8328 8365 8374 8375 8377 8381 8382 8385 8386 8400 8401 8402 8404 8407 8408 8423 |
| | 8425 8426 8431 8436 8454 8455 8458 8459 8463 8472 8476 8487 8489 8490 8491 8492 8509 8513 8514 8516 |
| | 8517 8531 8542 8545 8546 8547 8549 8551 8552 8555 8564 8565 8568 8570 8574 8575 8576 8578 8579 8580 |
| | 8588 8591 8601 8603 8604 8606 8609 8613 8616 8617 8618 8622 8627 8640 8641 8642 8644 8645 8647 8649 |
| | 8653 8654 8656 8657 8659 8662 8663 8664 8667 8669 8672 8673 8678 8688 8689 8691 8693 8700 8705 8706 |
| | 8709 8710 8713 8715 8718 8729 8739 8741 8743 8746 8747 8748 8753 8759 8764 8776 8777 8779 8781 8782 |
| | 8785 8786 8791 8796 8813 8815 8818 8819 8822 8832 8833 8839 8841 8842 8844 8847 8848 8853 8855 8856 |
| | 8857 8859 8866 8869 8871 8877 8880 8881 8883 8884 8892 8898 8924 8933 8939 8953 8954 8955 8957 8958 |
| | 8960 8968 8970 8977 9003 9004 9005 9006 9007 9040 9041 9044 9045 9046 9047 9048 9049 9051 9052 9072 |
| | 9075 9076 9077 9079 9080 9082 9083 9086 9088 9090 9093 9108 9113 9114 9116 9117 9118 9120 9121 9122 |
| | 9124 9125 9127 9128 9130 9133 9154 9156 9160 9162 9164 9167 9168 9169 9172 9173 9174 9176 9180 9184 |
| | 9199 9200 9201 9202 9204 9206 9249 9264 9270 9298 9299 9301 9304 9315 9319 9320 9321 9325 9326 9327 |
| | 9329 9330 9331 9335 9338 9343 9344 9345 9349 9355 9356 9357 9358 9359 9360 9362 9363 9364 9366 9371 |
| | 9377 9380 9382 9384 9385 9387 9392 9393 9395 9396 9399 9400 9404 9410 9415 9417 9419 9422 9424 9425 |
| | 9430 9437 9443 9445 9451 9452 9454 9455 9456 9457 9458 9460 9461 9470 9471 9478 9480 9481 9483 9484 |
| | 9486 9487 9488 9492 9493 9495 9497 9499 9500 9503 9516 9522 9525 9526 9527 9529 9530 9531 9533 9534 |
| | 9537 9543 9547 9550 9552 9553 9555 9557 9561 9562 9563 9571 9574 9575 9576 9577 9578 9579 9580 9583 |
| | 9589 9590 9593 9597 9598 9599 9600 9601 9602 9603 9607 9608 9611 9612 9614 9620 9623 9624 9626 9627 |
| | 9631 9633 9638 9667 9669 9670 9686 9688 9712 9722 9724 9730 9732 9744 9748 9755 9758 9763 9765 9768 |
| | 9771 9772 9776 9781 9782 9784 9785 9788 9789 9790 9793 9796 9799 9805 9806 9808 9809 9812 9813 9814 |
| | 9816 9818 9819 9821 9824 9829 9837 9841 9845 9846 9847 9849 9851 9853 9854 9862 9865 9868 9870 9872 |
| | 9885 9888 9891 9892 9896 9898 9899 9900 9901 9903 9904 9905 9906 9926 9929 9930 9931 9932 9934 9935 |
| | 9936 9939 9940 9941 9963 9964 9966 9969 9970 9973 9975 9977 9980 9982 9983 10002 10003 10006 10013 |
| | 10014 10015 10018 10021 10026 10038 10041 10044 10046 10048 10058 10074 10108 10111 10122 10133 10136 |
| | 10138 10139 10140 10143 10145 10149 10150 10152 10154 10158 10159 10168 10170 10172 10173 10175 10179 |
| | 10181 10186 10188 10192 10193 10194 10195 10196 10197 10200 10205 10207 10209 10213 10214 10215 10216 |
| | 10218 10219 10220 10221 10222 10224 10226 10227 10228 10230 10234 10236 10239 10240 10243 10244 10246 |
| | 10248 10249 10251 10252 10253 10255 10256 10258 10260 10262 10263 10267 10271 10273 10274 10275 10279 |
| | 10283 10287 10289 10291 10295 10297 10300 10301 10303 10308 10309 10310 10315 10316 10317 10318 10321 |
| | 10329 10331 10332 10336 10339 10345 10346 10347 10349 10355 10357 10362 10363 10366 10369 10371 10372 |
| | 10374 10375 10378 10384 10391 10393 10403 10406 10407 10409 10410 10412 10413 10416 10417 10419 10424 |
| | 10427 10430 10433 10442 10444 10447 10449 10452 10462 |
| 171: | 6919 6922 6926 6930 6932 6933 6946 6948 6950 6953 6955 6972 6994 7027 7028 7030 7033 7035 7044 |
| | 7047 7049 7050 7051 7054 7063 7067 7072 7073 7077 7080 7083 7085 7088 7100 7101 7106 7112 7116 7117 |
| | 7118 7124 7128 7129 7130 7132 7134 7136 7137 7138 7150 7153 7154 7156 7157 7175 7338 7368 7394 7396 |
| | 7398 7401 7403 7412 7430 7431 7433 7438 7443 7445 7448 7454 7455 7467 7469 7472 7473 7478 7479 7484 |
| | 7493 7495 7499 7500 7501 7502 7514 7518 7519 7523 7524 7526 7527 7535 7539 7544 7546 7547 7549 7550 |
| | 7552 7553 7560 7562 7578 7585 7671 7680 7682 7692 7698 7700 7702 7717 7720 7722 7740 7743 7758 7785 |
| | 7795 7815 7819 7820 7830 7831 7833 7836 7838 7841 7848 7866 7868 7869 7871 7873 7874 7875 7876 7883 |
| | 7898 7899 7903 7904 7905 7906 7908 7910 7921 7925 7934 7937 7938 7941 7942 7946 7967 7974 7977 7980 |
| | 7984 7985 8011 8012 8016 8019 8024 8041 8044 8047 8049 8051 8056 8060 8061 8062 8070 8072 8077 8079 |
| | 8081 8083 8085 8104 8132 8166 8169 8172 8174 8175 8178 8185 8310 8365 8374 8375 8377 8381 8382 8400 |
| | 8402 8404 8407 8408 8419 8423 8425 8426 8431 8436 8454 8455 8457 8458 8459 8463 8484 8486 8487 8489 |
| | 8490 8491 8492 8509 8513 8514 8516 8517 8542 8545 8546 8547 8549 8564 8565 8568 8570 8574 8576 8578 |
| | 8580 8583 8584 8588 8589 8591 8601 8603 8604 8606 8608 8609 8613 8616 8618 8622 8627 8634 8637 8640 |
| | 8641 8642 8644 8645 8647 8649 8653 8656 8657 8658 8659 8662 8663 8664 8667 8669 8672 8673 8678 8688 |
| | 8691 8693 8694 8700 8701 8705 8706 8709 8710 8713 8715 8718 8737 8739 8741 8742 8743 8746 8747 8748 |
| | 8753 8759 8764 8768 8776 8777 8779 8781 8782 8785 8786 8791 8796 8813 8815 8818 8819 8822 8837 8839 |
| | 8841 8842 8844 8847 8853 8855 8856 8857 8861 8865 8866 8869 8870 8871 8880 8881 8883 8884 8928 8931 |
| | 8933 8939 8949 8957 8960 8968 8970 8977 9003 9004 9005 9007 9020 9040 9041 9044 9045 9046 9047 9048 |
| | 9049 9051 9052 9072 9075 9076 9077 9079 9080 9082 9083 9086 9088 9090 9093 9108 9113 9114 9116 9117 |
| | 9118 9120 9121 9122 9124 9125 9127 9128 9130 9133 9154 9156 9160 9162 9164 9167 9168 9169 9172 9173 |
| | 9174 9176 9180 9184 9199 9200 9201 9202 9204 9206 9264 9272 9280 9297 9298 9301 9304 9319 9321 9325 |
| | 9326 9327 9329 9330 9335 9345 9349 9356 9357 9359 9360 9361 9362 9363 9364 9366 9371 9380 9382 9384 |
| | 9385 9387 9392 9393 9395 9399 9400 9402 9404 9408 9410 9412 9413 9415 9417 9419 9422 9424 9425 9435 |
| | 9437 9443 9445 9451 9452 9454 9455 9456 9457 9460 9461 9465 9478 9479 9480 9481 9483 9484 9486 9487 |
| | 9488 9492 9493 9497 9499 9500 9503 9516 9522 9525 9526 9527 9529 9530 9531 9533 9534 9537 9538 9543 |
| | 9547 9550 9552 9553 9555 9557 9561 9563 9571 9574 9575 9576 9577 9578 9579 9580 9582 9583 9598 9599 |
| | 9601 9602 9603 9606 9607 9608 9611 9612 9614 9623 9624 9626 9627 9631 9633 9656 9660 9662 9668 9684 |
| | 9707 9709 9730 9732 9765 9768 9771 9772 9776 9788 9789 9790 9793 9796 9807 9808 9816 9818 9819 9821 |
| | 9824 9845 9846 9847 9849 9851 9853 9854 9862 9865 9868 9870 9872 9885 9888 9891 9892 9896 9898 9899 |
| | 9900 9901 9903 9904 9905 9906 9929 9930 9931 9932 9934 9935 9936 9939 9940 9941 9964 9966 9969 9970 |
| | 9973 9975 9977 9980 9982 9983 10002 10013 10015 10018 10021 10026 10038 10041 10044 10046 10048 10074 |
| | 10077 10104 10106 10108 10112 10122 10126 10130 10133 10136 10138 10139 10140 10143 10145 10148 10149 |
| | 10152 10154 10155 10158 10159 10161 10164 10166 10167 10168 10170 10172 10173 10175 10179 10181 10186 |
| | 10188 10192 10193 10194 10195 10196 10197 10199 10200 10205 10207 10209 10213 10214 10215 10216 10218 |
| | 10220 10221 10222 10224 10225 10226 10227 10228 10230 10232 10234 10236 10239 10240 10243 10244 10246 |
| | 10248 10249 10252 10253 10255 10256 10258 10260 10262 10263 10267 10272 10273 10274 10275 10283 10287 |
| | 10289 10291 10295 10297 10301 10303 10308 10309 10310 10315 10316 10317 10318 10321 10323 10329 10331 |
| | 10332 10336 10339 10346 10347 10349 10363 10366 10369 10371 10372 10374 10375 10378 10384 10403 10406 |
| | 10407 10409 10410 10412 10413 10416 10417 10419 10420 10424 10427 10430 10433 10442 10444 10447 10449 |
| | 10452 10462 |
| 172: | 7010 7053 7161 7293 7790 7823 7917 8065 8129 8917 8925 9042 9339 9490 9649 9804 10107 10147 10405 |
| 173: | 6974 6978 7064 7160 7223 7238 7364 7609 7773 7774 7845 7880 7911 7931 7947 8111 8113 8115 8145 |
| | 8210 8250 8264 8295 8296 8367 8495 8504 8523 8614 8679 8766 8793 8906 8963 9137 9406 9658 9681 9762 |
| | 9791 9794 10035 10065 10119 10129 10146 10151 10278 10359 10468 |
| 174: | 6964 7097 7110 7425 7591 7617 7710 7755 7991 8058 8091 8139 8326 8340 8346 8480 8541 8543 8765 |
| | 8773 8784 8809 8834 8915 9059 9097 9150 9276 9286 9650 9655 9836 9943 9997 10052 10134 10141 10338 |
| 175: | 7390 7580 8153 8508 8572 8695 9073 9084 9306 9333 9506 9588 9842 10010 10103 10206 10434 |
| 176: | 6918 6938 6966 6975 6976 6977 6996 6999 7000 7014 7020 7032 7060 7086 7115 7121 7123 7177 7178 |
| | 7185 7188 7191 7233 7237 7261 7262 7263 7271 7296 7298 7302 7306 7307 7317 7326 7337 7346 7349 7352 |
| | 7374 7376 7377 7378 7380 7383 7406 7409 7415 7426 7429 7437 7453 7464 7477 7504 7510 7515 7530 7574 |
| | 7576 7577 7588 7593 7594 7602 7614 7621 7623 7626 7654 7677 7679 7745 7797 7813 7877 7953 7954 7993 |
| | 8023 8032 8036 8059 8069 8088 8089 8093 8095 8098 8099 8150 8152 8177 8180 8209 8231 8235 8269 8281 |
| | 8290 8291 8294 8320 8357 8362 8363 8369 8379 8380 8388 8429 8438 8449 8453 8456 8500 8522 8563 8586 |
| | 8587 8593 8598 8612 8625 8631 8692 8714 8720 8725 8754 8757 8761 8772 8774 8792 8806 8808 8816 8826 |
| | 8843 8851 8854 8860 8872 8886 8888 8895 8900 8934 8951 8964 8972 8980 8981 8982 8983 8984 8985 8988 |
| | 8990 9008 9010 9011 9015 9016 9037 9054 9055 9064 9081 9103 9109 9123 9134 9145 9146 9151 9157 9158 |
| | 9177 9183 9188 9193 9223 9227 9228 9229 9230 9236 9237 9242 9243 9251 9254 9255 9256 9259 9267 9271 |
| | 9273 9290 9300 9303 9334 9347 9353 9369 9374 9420 9421 9423 9426 9446 9475 9514 9519 9523 9524 9554 |
| | 9556 9558 9559 9560 9581 9641 9644 9671 9673 9676 9696 9697 9698 9699 9700 9701 9702 9706 9716 9726 |
| | 9728 9729 9731 9733 9734 9752 9759 9766 9769 9840 9857 9859 9863 9873 9879 9890 9894 9895 9897 9907 |
| | 9909 9911 9915 9924 9927 9942 9954 9957 9965 9967 9990 9993 9998 10004 10016 10017 10061 10064 10069 |
| | 10070 10075 10096 10127 10131 10142 10171 10174 10203 10208 10237 10241 10259 10264 10276 10281 10292 |
| | 10304 10311 10312 10322 10422 10425 10437 10439 10457 10459 |
| 177: | 6925 6928 6934 6985 7046 7104 7108 7135 7163 7208 7308 7311 7402 7414 7631 7633 7767 7844 8008 |
| | 8073 8082 8200 8255 8282 8288 8298 8398 8485 8527 8535 8548 8557 8605 8708 8798 8811 8831 9017 9026 |
| | 9030 9211 9215 9284 9310 9350 9427 9462 9548 9632 9825 9848 9922 10049 10128 10162 10198 10266 10268 |
| | 10387 10411 |
| 178: | 6919 6922 6926 6930 6932 6933 6946 6948 6950 6953 6955 6957 6971 6972 6986 6991 6994 6998 7011 |
| | 7027 7028 7030 7033 7035 7044 7047 7049 7050 7051 7054 7056 7063 7067 7072 7077 7080 7083 7085 7088 |
| | 7093 7100 7101 7106 7112 7116 7117 7118 7124 7128 7129 7130 7132 7134 7136 7137 7138 7150 7153 7154 |
| | 7156 7157 7162 7164 7175 7216 7269 7327 7386 7394 7396 7398 7401 7403 7407 7412 7417 7430 7431 7433 |
| | 7438 7443 7445 7448 7455 7467 7469 7470 7472 7473 7478 7479 7485 7490 7493 7494 7495 7497 7499 7500 |
| | 7501 7502 7514 7518 7519 7523 7524 7526 7527 7539 7541 7544 7546 7547 7549 7550 7552 7560 7562 7566 |
| | 7578 7585 7756 7761 7762 7763 7781 7786 7792 7804 7817 7830 7831 7833 7834 7836 7838 7840 7841 7849 |
| | 7866 7868 7869 7871 7873 7875 7879 7881 7884 7898 7899 7903 7904 7905 7906 7908 7910 7914 7921 7925 |
| | 7934 7937 7938 7941 7942 7943 7944 7946 7951 7965 7967 7974 7977 7980 7984 7985 8005 8011 8012 8016 |
| | 8019 8024 8041 8044 8047 8049 8051 8064 8066 8072 8077 8079 8081 8083 8085 8147 8169 8170 8172 8174 |
| | 8175 8178 8224 8233 8310 8328 8365 8374 8375 8377 8381 8382 8385 8386 8400 8401 8402 8404 8407 8408 |
| | 8423 8425 8426 8431 8436 8454 8455 8458 8459 8463 8472 8476 8487 8489 8490 8491 8492 8509 8513 8514 |
| | 8516 8517 8531 8542 8545 8546 8547 8549 8551 8552 8555 8564 8565 8568 8570 8574 8575 8576 8578 8579 |
| | 8580 8588 8591 8601 8603 8604 8606 8609 8613 8616 8617 8618 8622 8627 8639 8640 8641 8642 8644 8645 |
| | 8647 8649 8653 8654 8656 8657 8659 8662 8663 8664 8667 8669 8672 8673 8678 8688 8689 8691 8693 8700 |
| | 8705 8706 8709 8710 8713 8715 8718 8729 8739 8741 8743 8746 8747 8748 8753 8759 8764 8776 8777 8779 |
| | 8781 8782 8785 8786 8791 8796 8813 8815 8818 8819 8822 8832 8833 8839 8841 8842 8844 8847 8848 8853 |
| | 8855 8856 8857 8859 8866 8869 8871 8877 8880 8881 8883 8884 8887 8892 8898 8924 8933 8939 8953 8954 |
| | 8955 8957 8958 8960 8968 8970 8977 9003 9004 9005 9006 9007 9040 9041 9044 9045 9046 9047 9048 9049 |
| | 9051 9052 9072 9075 9076 9077 9079 9080 9082 9083 9086 9088 9090 9093 9108 9113 9114 9116 9117 9118 |
| | 9120 9121 9122 9124 9125 9127 9128 9130 9133 9154 9156 9160 9162 9164 9167 9168 9169 9172 9173 9174 |
| | 9176 9180 9184 9199 9200 9201 9202 9204 9206 9249 9264 9270 9298 9299 9301 9304 9315 9319 9320 9321 |
| | 9325 9326 9327 9329 9330 9331 9335 9338 9343 9344 9345 9349 9355 9356 9357 9358 9359 9360 9362 9363 |
| | 9364 9366 9371 9377 9380 9382 9384 9385 9387 9392 9393 9395 9396 9399 9400 9404 9410 9415 9417 9419 |
| | 9422 9424 9425 9430 9443 9445 9451 9452 9454 9455 9456 9457 9458 9460 9461 9470 9471 9478 9480 9481 |
| | 9483 9484 9486 9487 9488 9492 9493 9495 9497 9499 9500 9503 9516 9522 9525 9526 9527 9529 9530 9531 |
| | 9533 9534 9537 9543 9547 9550 9552 9553 9555 9557 9561 9562 9563 9571 9574 9575 9576 9577 9578 9579 |
| | 9580 9583 9589 9590 9593 9597 9598 9599 9600 9601 9602 9603 9607 9608 9611 9612 9614 9620 9623 9624 |
| | 9626 9627 9631 9633 9638 9667 9669 9670 9686 9688 9712 9722 9724 9730 9732 9744 9748 9755 9758 9763 |
| | 9765 9768 9771 9772 9776 9781 9782 9784 9785 9788 9789 9790 9793 9796 9799 9805 9806 9808 9809 9812 |
| | 9813 9814 9816 9818 9819 9821 9824 9829 9837 9841 9845 9846 9847 9849 9851 9853 9854 9862 9865 9868 |
| | 9870 9872 9885 9888 9891 9892 9896 9898 9899 9900 9901 9903 9904 9905 9906 9926 9929 9930 9931 9932 |
| | 9934 9935 9936 9939 9940 9941 9963 9964 9966 9969 9970 9973 9975 9977 9980 9982 9983 10002 10003 10006 |
| | 10013 10014 10015 10018 10021 10026 10038 10041 10044 10046 10048 10058 10074 10108 10111 10122 10133 |
| | 10136 10138 10139 10140 10143 10145 10149 10150 10152 10154 10158 10159 10168 10170 10172 10173 10175 |
| | 10179 10181 10186 10188 10192 10193 10194 10195 10196 10200 10205 10207 10209 10213 10214 10215 10216 |
| | 10218 10219 10220 10221 10222 10224 10226 10227 10228 10230 10234 10236 10239 10240 10243 10244 10246 |
| | 10248 10249 10251 10252 10253 10255 10256 10258 10260 10262 10263 10267 10271 10274 10275 10279 10283 |
| | 10287 10289 10291 10295 10297 10300 10301 10303 10308 10309 10310 10315 10316 10317 10318 10321 10329 |
| | 10331 10332 10336 10339 10345 10346 10347 10349 10355 10357 10362 10363 10366 10369 10371 10372 10374 |
| | 10375 10378 10384 10391 10393 10403 10406 10407 10409 10410 10412 10413 10416 10417 10419 10424 10427 |
| | 10430 10433 10442 10444 10447 10449 10452 10462 |
| 179: | 6940 6943 7005 7015 7039 7040 7043 7061 7099 7181 7183 7186 7189 7258 7272 7282 7285 7356 7439 |
| | 7447 7451 7521 7525 7533 7535 7550 7553 7555 7558 7592 7598 7613 7640 7665 7687 7696 7768 7814 7851 |
| | 7865 7927 8052 8054 8057 8114 8141 8149 8173 8352 8356 8373 8422 8430 8446 8451 8551 8553 8566 8716 |
| | 8726 8730 8787 8801 8803 8804 8825 8830 8873 8902 8909 8912 8914 8916 8918 8919 8920 8973 8974 8991 |
| | 8994 9009 9023 9089 9099 9101 9111 9191 9194 9203 9245 9247 9250 9252 9289 9340 9351 9370 9390 9447 |
| | 9449 9494 9528 9535 9618 9647 9657 9663 9674 9694 9717 9741 9779 9833 9949 10023 10027 10029 10080 |
| | 10083 10137 10184 10185 10187 10189 10201 10210 10313 10328 |
| 180: | 6973 6989 6993 7004 7037 7058 7070 7092 7139 7220 7324 7371 7379 7488 7554 7620 7676 7726 7856 |
| | 7888 7945 7952 7997 8078 8167 8249 8468 8577 8696 8751 8760 8767 9163 9192 9232 9261 9268 9302 9336 |
| | 9482 9635 9661 9677 9703 9735 9753 9815 9831 9947 10043 10059 10062 10091 10182 10288 10333 10436 |
| | 10471 |
| 181: | 7025 7252 7463 7498 7777 7779 7780 8619 8623 9401 9414 9418 9880 9882 10350 10353 10463 |
| 182: | 7026 7279 7393 7411 7651 7753 7864 8300 8660 8932 8940 9058 9159 9296 9636 9678 10019 10176 |
| 183: | 6952 6968 6979 6984 7018 7019 7024 7057 7151 7316 7330 7355 7400 7405 7432 7442 7492 7529 7658 |
| | 7765 7812 7839 7862 7990 8022 8028 8031 8035 8037 8122 8160 8230 8355 8630 8648 8727 8752 8927 8950 |
| | 8961 9115 9178 9218 9453 9496 9594 9617 9659 9713 9736 9820 9843 9850 9945 9976 9978 10125 10190 10269 |
| | 10314 10469 |
| 184: | 6917 6920 6924 6942 6961 6963 6965 6967 6970 6981 6987 7001 7003 7007 7008 7016 7022 7023 7062 |
| | 7069 7076 7094 7103 7105 7109 7111 7114 7131 7133 7141 7145 7146 7148 7149 7152 7168 7169 7171 7172 |
| | 7174 7179 7182 7193 7194 7196 7197 7198 7199 7201 7203 7204 7206 7210 7214 7217 7218 7222 7226 7227 |
| | 7229 7231 7232 7235 7243 7245 7246 7247 7249 7250 7254 7255 7256 7257 7259 7260 7264 7275 7277 7280 |
| | 7281 7284 7286 7287 7288 7290 7294 7295 7304 7310 7312 7314 7315 7318 7319 7323 7329 7333 7340 7342 |
| | 7343 7344 7348 7351 7357 7359 7362 7365 7366 7367 7370 7373 7375 7395 7404 7408 7410 7420 7423 7436 |
| | 7441 7444 7446 7449 7456 7457 7458 7460 7461 7462 7471 7489 7516 7543 7561 7575 7582 7583 7584 7586 |
| | 7599 7601 7603 7606 7608 7610 7612 7615 7618 7619 7622 7625 7629 7632 7635 7636 7639 7642 7644 7645 |
| | 7648 7649 7650 7653 7662 7664 7667 7669 7670 7673 7674 7683 7684 7688 7691 7693 7695 7697 7699 7701 |
| | 7704 7705 7714 7715 7716 7723 7732 7736 7737 7739 7741 7752 7759 7760 7776 7778 7789 7798 7799 7801 |
| | 7802 7805 7807 7808 7810 7816 7821 7822 7824 7826 7828 7829 7832 7835 7837 7847 7850 7852 7853 7855 |
| | 7858 7861 7867 7872 7887 7889 7891 7892 7893 7896 7900 7919 7920 7922 7924 7926 7928 7929 7930 7933 |
| | 7940 7950 7956 7957 7958 7959 7960 7962 7964 7966 7973 7976 7978 7983 7986 7988 7992 7995 7996 7998 |
| | 7999 8000 8001 8002 8003 8004 8007 8014 8015 8018 8020 8021 8025 8026 8027 8030 8038 8043 8048 8050 |
| | 8075 8080 8090 8100 8102 8106 8109 8110 8119 8121 8124 8125 8133 8135 8136 8157 8158 8159 8163 8168 |
| | 8176 8179 8184 8186 8189 8193 8194 8197 8208 8213 8214 8215 8216 8217 8219 8234 8237 8239 8240 8241 |
| | 8242 8243 8244 8247 8252 8260 8262 8267 8268 8271 8272 8274 8275 8276 8278 8280 8284 8285 8286 8292 |
| | 8301 8303 8304 8305 8311 8317 8319 8322 8323 8325 8329 8330 8331 8332 8333 8342 8343 8345 8348 8349 |
| | 8351 8361 8364 8366 8378 8384 8397 8403 8406 8411 8416 8433 8461 8479 8493 8499 8502 8520 8525 8526 |
| | 8530 8532 8533 8540 8544 8554 8569 8581 8582 8610 8615 8633 8661 8666 8668 8671 8682 8684 8698 8699 |
| | 8703 8711 8719 8722 8732 8734 8738 8749 8755 8758 8783 8788 8790 8797 8799 8810 8814 8820 8821 8824 |
| | 8828 8835 8845 8850 8874 8876 8903 8905 8908 8910 8911 8913 8936 8937 8941 8943 8966 8969 8978 8986 |
| | 8996 9012 9018 9019 9021 9022 9024 9027 9028 9031 9035 9036 9038 9061 9065 9067 9068 9069 9070 9074 |
| | 9078 9091 9095 9100 9102 9106 9107 9112 9129 9131 9135 9138 9139 9140 9142 9148 9149 9152 9155 9161 |
| | 9165 9171 9182 9187 9190 9195 9196 9197 9198 9205 9208 9210 9214 9216 9217 9219 9225 9260 9274 9277 |
| | 9305 9332 9346 9348 9354 9365 9383 9391 9432 9434 9438 9440 9468 9472 9477 9491 9498 9501 9505 9507 |
| | 9509 9510 9512 9513 9515 9517 9541 9549 9567 9568 9585 9613 9625 9630 9634 9664 9680 9683 9685 9689 |
| | 9691 9692 9708 9711 9719 9720 9721 9742 9743 9749 9754 9760 9800 9832 9867 9884 9913 9914 9917 9919 |
| | 9920 9923 9928 9944 9948 9950 9953 9956 9968 9972 9974 9981 9987 9994 9995 9996 10011 10024 10028 |
| | 10032 10033 10045 10051 10067 10068 10071 10072 10073 10076 10078 10079 10081 10088 10090 10092 10093 |
| | 10094 10109 10113 10123 10144 10156 10163 10202 10223 10235 10254 10294 10302 10305 10307 10324 10341 |
| | 10343 10344 10352 10354 10356 10358 10377 10382 10383 10386 10388 10390 10392 10394 10396 10423 10426 |
| | 10431 10432 10438 10445 10448 10451 10454 10456 10458 10461 10464 10466 10467 10472 |
| 185: | 6923 6929 6931 6949 6954 6958 6983 7031 7036 7055 7065 7079 7096 7140 7176 7195 7202 7205 7212 |
| | 7213 7240 7268 7270 7276 7301 7303 7321 7322 7331 7332 7363 7372 7385 7466 7476 7483 7486 7487 7491 |
| | 7496 7508 7545 7548 7573 7589 7607 7628 7634 7641 7657 7666 7689 7694 7703 7708 7724 7764 7772 7803 |
| | 7846 7859 7860 7886 7890 7895 7897 7913 7982 8013 8017 8039 8046 8067 8068 8074 8084 8092 8118 8143 |
| | 8155 8156 8162 8181 8188 8192 8202 8205 8220 8228 8245 8246 8283 8289 8297 8307 8308 8316 8339 8353 |
| | 8354 8410 8413 8421 8437 8462 8488 8496 8497 8512 8519 8560 8573 8607 8611 8621 8643 8651 8652 8686 |
| | 8763 8771 8780 8800 8802 8805 8864 8868 8891 8923 8929 8971 9014 9032 9034 9039 9043 9060 9066 9096 |
| | 9175 9209 9213 9224 9239 9244 9262 9263 9265 9275 9279 9281 9293 9294 9308 9381 9386 9388 9397 9398 |
| | 9439 9442 9485 9508 9521 9542 9572 9596 9642 9648 9666 9704 9710 9715 9725 9727 9739 9740 9775 9835 |
| | 9856 9860 9861 9871 9875 9876 9887 9893 9902 9938 9960 9979 9991 9992 10000 10005 10008 10022 10039 |
| | 10053 10057 10082 10102 10114 10116 10120 10191 10217 10231 10284 10298 10299 10306 10320 10326 10365 |
| | 10381 10402 10441 10443 |
| 186: | 6936 6990 7071 7144 7147 7209 7382 7482 7738 7994 8218 8261 8391 8399 8447 8707 8823 8942 9085 |
| | 9450 9511 9646 9693 10054 10097 10265 10351 10379 |
| 187: | 6921 6927 6935 6937 6941 6945 6947 6951 6956 6959 6960 6962 6969 6980 6982 6988 6992 6995 6997 |
| | 7006 7009 7012 7013 7017 7021 7034 7038 7041 7042 7045 7052 7059 7066 7068 7074 7075 7084 7087 7089 |
| | 7090 7098 7102 7107 7113 7120 7122 7126 7127 7143 7155 7158 7159 7166 7167 7170 7173 7184 7190 7192 |
| | 7207 7211 7215 7221 7224 7225 7230 7234 7236 7241 7251 7265 7266 7267 7273 7274 7278 7283 7289 7292 |
| | 7299 7300 7305 7309 7313 7320 7325 7328 7335 7336 7339 7345 7347 7350 7353 7354 7358 7360 7361 7369 |
| | 7381 7384 7387 7388 7389 7391 7392 7413 7416 7419 7421 7422 7427 7428 7434 7435 7440 7450 7452 7468 |
| | 7474 7475 7480 7481 7503 7505 7507 7513 7517 7520 7522 7528 7537 7542 7551 7556 7557 7559 7563 7568 |
| | 7570 7571 7579 7587 7597 7605 7630 7638 7647 7656 7659 7663 7668 7672 7675 7681 7690 7713 7718 7719 |
| | 7721 7725 7728 7734 7742 7744 7747 7750 7754 7757 7771 7775 7782 7783 7784 7787 7791 7793 7794 7796 |
| | 7800 7806 7809 7811 7818 7825 7842 7843 7857 7863 7870 7878 7882 7885 7901 7912 7915 7916 7918 7923 |
| | 7932 7936 7939 7949 7961 7963 7968 7970 7971 7979 7981 7987 8006 8009 8010 8029 8033 8034 8040 8045 |
| | 8055 8063 8071 8076 8086 8087 8094 8097 8105 8107 8108 8112 8117 8120 8126 8127 8128 8130 8131 8134 |
| | 8138 8142 8146 8148 8151 8154 8161 8165 8171 8182 8190 8191 8195 8196 8198 8199 8201 8203 8206 8207 |
| | 8212 8221 8222 8223 8226 8227 8229 8232 8236 8238 8248 8251 8254 8256 8257 8259 8261 8263 8265 8266 |
| | 8270 8279 8287 8293 8299 8302 8306 8309 8313 8315 8321 8324 8327 8334 8335 8336 8338 8341 8344 8347 |
| | 8350 8358 8359 8360 8372 8376 8383 8387 8389 8390 8392 8394 8395 8405 8409 8415 8418 8424 8427 8428 |
| | 8434 8435 8439 8440 8441 8444 8445 8452 8464 8465 8467 8469 8474 8475 8478 8481 8482 8483 8501 8507 |
| | 8510 8511 8515 8518 8521 8528 8529 8537 8538 8550 8559 8562 8567 8585 8595 8600 8602 8620 8635 8636 |
| | 8638 8646 8650 8655 8665 8670 8675 8676 8677 8680 8681 8683 8685 8690 8712 8721 8723 8724 8728 8733 |
| | 8735 8736 8740 8744 8745 8756 8762 8769 8770 8775 8778 8789 8795 8807 8827 8840 8846 8849 8858 8862 |
| | 8867 8875 8878 8879 8882 8890 8894 8896 8897 8899 8901 8907 8921 8922 8926 8935 8945 8946 8947 8948 |
| | 8952 8959 8962 8965 8975 8976 8979 8989 8992 8993 8997 8998 8999 9000 9001 9002 9025 9029 9033 9053 |
| | 9062 9071 9087 9094 9098 9105 9126 9141 9143 9144 9147 9153 9166 9170 9179 9181 9186 9207 9212 9220 |
| | 9221 9222 9226 9231 9235 9238 9241 9246 9248 9258 9266 9269 9278 9282 9283 9285 9288 9295 9307 9311 |
| | 9313 9314 9316 9322 9323 9324 9337 9341 9342 9352 9376 9378 9379 9389 9394 9411 9416 9428 9459 9464 |
| | 9489 9502 9518 9520 9532 9539 9544 9546 9551 9564 9565 9566 9569 9570 9573 9584 9586 9587 9592 9595 |
| | 9605 9609 9610 9615 9619 9621 9622 9628 9629 9637 9639 9640 9645 9651 9652 9653 9654 9665 9672 9679 |
| | 9682 9687 9690 9695 9705 9718 9737 9747 9751 9757 9767 9770 9773 9774 9778 9786 9792 9795 9797 9798 |
| | 9802 9811 9817 9823 9826 9827 9828 9834 9839 9844 9852 9855 9858 9864 9869 9874 9877 9878 9886 9889 |
| | 9908 9910 9916 9918 9921 9925 9933 9951 9952 9955 9958 9959 9961 9962 9984 9985 9986 9989 9999 10001 |
| | 10007 10020 10025 10030 10034 10036 10037 10042 10047 10050 10055 10056 10063 10066 10085 10086 10089 |
| | 10095 10098 10099 10100 10101 10105 10110 10117 10118 10121 10124 10132 10135 10157 10160 10165 10169 |
| | 10178 10180 10204 10212 10233 10238 10242 10245 10247 10250 10257 10261 10270 10277 10282 10286 10290 |
| | 10293 10296 10319 10325 10330 10334 10335 10340 10342 10348 10370 10373 10376 10380 10385 10389 10397 |
| | 10399 10401 10415 10418 10421 10428 10429 10435 10440 10446 10450 10455 10460 10465 10470 |
| 188: | 7002 7091 7200 7219 7297 7334 7418 7424 7465 7590 7627 7707 7731 7733 7735 7902 7935 7975 8204 |
| | 8225 8253 8318 8337 8393 8412 8432 8450 8571 8597 8750 8836 8838 9050 9057 9110 9119 9132 9185 9240 |
| | 9291 9309 9328 9368 9675 9738 9912 9937 9971 10009 10012 10031 10040 10060 10177 10183 10211 10337 |
| | 10360 10361 |
| 189: | 7078 7082 7142 7291 7341 7399 7596 7907 8103 8211 8258 8314 8674 8717 8812 8829 8885 8889 8938 |
| | 8995 9013 9056 9063 9136 9189 9409 9476 9604 9746 9756 9761 9764 9783 9810 9866 9946 10115 10153 10229 |
| | 10327 10414 |
| 190: | 7078 7082 7142 7291 7341 7399 7596 7907 8103 8211 8258 8314 8674 8717 8812 8829 8885 8889 8938 |
| | 8995 9013 9056 9063 9136 9189 9409 9476 9604 9746 9756 9761 9764 9783 9810 9866 9946 10115 10153 10229 |
| | 10327 10414 |
| 191: | 7228 7459 7531 7565 7709 7894 7948 7969 7972 8101 8277 8396 8460 8506 8524 8687 8852 8863 8904 |
| | 8930 8956 8987 9092 9292 9312 9444 9474 9536 9545 9616 9643 9723 9803 9881 9988 10084 10285 10368 |
| | 10453 |
| 192: | 6939 6944 7029 7048 7081 7095 7119 7125 7165 7180 7187 7239 7242 7244 7248 7253 7397 7506 7509 |
| | 7512 7532 7534 7536 7538 7540 7564 7567 7569 7572 7581 7595 7600 7604 7611 7616 7624 7637 7643 7646 |
| | 7652 7655 7660 7661 7678 7685 7686 7706 7711 7712 7727 7729 7730 7746 7748 7749 7751 7766 7769 7770 |
| | 7788 7827 7854 7909 7955 7989 8042 8053 8096 8116 8123 8137 8140 8144 8164 8183 8187 8273 8312 8368 |
| | 8370 8371 8414 8417 8420 8442 8443 8448 8466 8470 8471 8473 8477 8494 8498 8503 8505 8534 8536 8539 |
| | 8556 8558 8561 8590 8592 8594 8596 8599 8624 8626 8628 8629 8632 8697 8702 8704 8731 8794 8817 8893 |
| | 8944 8967 9104 9233 9234 9253 9257 9287 9317 9318 9367 9372 9373 9375 9403 9405 9407 9429 9431 9433 |
| | 9436 9441 9448 9463 9466 9467 9469 9473 9504 9540 9591 9714 9745 9750 9777 9780 9787 9801 9822 9830 |
| | 9838 9883 10087 10364 10367 10395 10398 10400 10404 10408 |
| 193: | 10485 10487 10490 10492 10514 10515 10521 10561 10580 10586 10591 10615 10638 10641 10642 10668 |
| | 10684 10691 10695 10738 10762 10777 10912 10982 11170 11273 11294 11295 11418 11624 11747 11807 12093 |
| | 12130 12322 12327 12357 12492 12521 12754 12791 12859 12971 13133 13158 13240 13385 13412 13476 13510 |
| | 13618 13808 13859 14057 14076 14081 14102 14103 14104 14118 14120 14135 14141 14155 14199 14201 14294 |
| | 14368 14375 14453 14478 14554 14560 14617 14701 14713 14739 14747 14829 14858 14974 14980 14995 15003 |
| | 15093 15161 15167 15168 15172 15198 15200 15206 15208 15226 15229 15233 15240 15242 15243 15274 15275 |
| | 15301 15306 15323 15378 15411 15469 15785 15788 15819 15832 15855 15917 15972 16015 16192 16425 16492 |
| | 16574 16595 16600 16639 16796 16800 16914 17016 17061 17108 17184 17229 17261 17364 17365 17368 17371 |
| 194: | 10501 10536 10538 10575 10581 10589 10607 10623 10697 10700 10726 10754 10768 10787 10790 10842 |
| | 10883 10909 10916 10921 10957 10965 10966 10984 11018 11033 11045 11054 11069 11098 11114 11119 11135 |
| | 11138 11222 11240 11303 11380 11485 11509 11531 11542 11606 11647 11665 11699 11753 11784 11792 11802 |
| | 11809 11810 11838 11844 11906 11910 11911 11912 11936 11953 11969 12018 12032 12033 12047 12051 12068 |
| | 12070 12123 12174 12196 12279 12287 12302 12314 12334 12384 12386 12393 12409 12415 12418 12444 12458 |
| | 12479 12499 12527 12569 12583 12591 12592 12604 12645 12683 12684 12686 12729 12738 12757 12760 12761 |
| | 12764 12788 12822 12833 12836 12903 12904 12908 12916 12923 12936 12954 12965 13013 13059 13063 13065 |
| | 13068 13070 13079 13083 13100 13140 13157 13168 13177 13194 13203 13211 13216 13218 13220 13221 13266 |
| | 13268 13296 13354 13358 13369 13370 13372 13375 13394 13424 13451 13600 13602 13653 13695 13763 13767 |
| | 13793 13799 13843 13931 13952 14017 14018 14038 14042 14067 14070 14085 14113 14126 14139 14185 14192 |
| | 14202 14208 14240 14256 14258 14276 14297 14298 14300 14302 14327 14347 14351 14362 14366 14486 14507 |
| | 14563 14565 14569 14589 14609 14621 14667 14670 14690 14696 14705 14720 14732 14744 14777 14778 14780 |
| | 14790 14792 14793 14859 14884 14952 14955 14958 14964 14967 14968 14975 15037 15052 15053 15068 15095 |
| | 15096 15121 15132 15154 15155 15178 15231 15318 15363 15464 15472 15558 15622 15646 15677 15694 15716 |
| | 15740 15769 15781 15807 15836 15849 15863 15866 15878 15897 15904 15936 15941 15943 15956 15967 16016 |
| | 16070 16071 16124 16132 16149 16153 16199 16200 16202 16230 16280 16289 16327 16465 16467 16479 16532 |
| | 16534 16567 16591 16604 16642 16645 16687 16688 16745 16761 16773 16810 16845 16894 16947 16959 16980 |
| | 17043 17050 17051 17074 17093 17242 17294 17351 |
| 195: | 10501 10536 10575 10581 10589 10607 10623 10697 10700 10726 10754 10768 10787 10790 10795 10842 |
| | 10883 10916 10925 10956 10957 10965 10984 11018 11045 11054 11058 11069 11098 11114 11119 11135 11137 |
| | 11138 11222 11240 11303 11380 11433 11475 11485 11509 11531 11606 11647 11665 11674 11699 11753 11781 |
| | 11784 11792 11802 11809 11810 11838 11844 11910 11911 11912 11936 11959 12018 12029 12032 12033 12051 |
| | 12065 12070 12123 12155 12174 12196 12208 12213 12279 12287 12302 12310 12314 12384 12386 12392 12393 |
| | 12409 12415 12444 12449 12450 12479 12499 12569 12592 12645 12673 12683 12684 12686 12729 12736 12757 |
| | 12760 12761 12764 12783 12788 12812 12833 12836 12903 12904 12908 12916 12936 12965 13013 13015 13027 |
| | 13059 13065 13068 13070 13079 13083 13089 13100 13140 13157 13166 13168 13177 13192 13194 13203 13211 |
| | 13220 13221 13266 13268 13296 13354 13355 13369 13370 13375 13382 13394 13424 13451 13600 13602 13653 |
| | 13662 13695 13763 13767 13793 13799 13843 13952 14017 14018 14038 14042 14060 14067 14070 14085 14111 |
| | 14113 14126 14185 14192 14202 14208 14222 14240 14256 14258 14276 14298 14302 14327 14332 14347 14351 |
| | 14366 14456 14486 14506 14507 14539 14569 14589 14609 14621 14670 14690 14720 14744 14777 14778 14790 |
| | 14792 14793 14884 14967 14975 15037 15068 15096 15107 15121 15132 15154 15155 15178 15231 15258 15287 |
| | 15318 15330 15363 15404 15464 15472 15475 15547 15558 15622 15646 15677 15694 15716 15740 15744 15769 |
| | 15781 15807 15833 15836 15849 15863 15866 15878 15897 15927 15936 15941 15943 15967 15973 15979 16016 |
| | 16070 16071 16124 16132 16149 16199 16200 16202 16222 16230 16280 16289 16327 16354 16380 16451 16465 |
| | 16467 16474 16479 16508 16532 16534 16567 16575 16591 16604 16642 16661 16687 16688 16745 16773 16810 |
| | 16845 16849 16855 16959 16980 17043 17050 17074 17093 17154 17159 17242 17294 17351 |
| 196: | 10622 10696 10699 10914 10961 11053 11097 11118 11169 11220 11311 11460 11484 11508 11754 12173 |
| | 12289 12395 12408 12481 12500 13072 13169 13477 13702 13970 13988 14142 14344 14407 14430 14694 14876 |
| | 14928 15182 15237 15467 15556 15835 16544 16619 16632 16847 17041 17069 17086 17346 |
| 197: | 10810 11024 11029 11268 11278 11523 12222 12366 12565 12866 12914 13132 13160 13386 13402 13470 |
| | 13513 13915 14119 14165 14358 15294 15606 15852 15954 15976 16405 |
| 198: | 10796 10800 10815 10847 10964 11325 11406 11607 11798 12266 12338 12455 12690 12873 13872 14194 |
| | 14796 14813 14921 15039 15478 15651 15676 15686 16115 16622 17286 17358 17359 |
| 199: | 10498 10625 10829 10854 10892 10924 11039 11083 11101 11179 11326 11327 11358 11388 11436 11471 |
| | 11503 11630 11683 11692 11694 11703 11762 11764 11765 11869 11888 11915 11975 11999 12175 12220 12323 |
| | 12342 12375 12401 12584 12654 12742 12855 12933 12935 13037 13077 13145 13282 13292 13435 13575 13664 |
| | 13701 13822 14107 14224 14290 14458 14782 14803 14825 14875 14904 14988 15166 15179 15246 15297 15425 |
| | 15436 15471 15584 15723 15795 15809 15848 16047 16087 16126 16154 16211 16223 16300 16321 16339 16480 |
| | 16521 16700 16718 16857 16865 16889 16921 16944 17045 17084 17136 17169 17197 17282 17327 |
| 200: | 10498 10625 10829 10854 10892 10924 11039 11083 11101 11164 11254 11326 11327 11358 11388 11436 |
| | 11471 11503 11692 11694 11703 11715 11762 11764 11765 11869 11888 11915 11940 11975 11999 12062 12175 |
| | 12323 12342 12375 12401 12654 12677 12742 12855 12889 12918 12991 13037 13145 13292 13435 13552 13575 |
| | 13664 13701 13822 14079 14220 14224 14458 14578 14682 14803 14875 14904 14988 15166 15246 15297 15425 |
| | 15476 15584 15723 15795 15809 15848 16156 16211 16223 16300 16339 16480 16521 16700 16857 16865 16889 |
| | 16898 16944 17045 17084 17136 17169 17197 17243 17282 17327 17361 |
| 201: | 10848 11066 11163 11246 11286 11724 11806 11926 12008 12094 12207 12526 12549 12832 13149 13244 |
| | 13495 13511 13523 13721 13806 13809 13973 14312 14575 14832 14840 14865 15083 15105 15120 15125 15138 |
| | 15213 15678 15712 15725 15890 15911 15951 16141 16161 16190 16378 16408 16524 16654 16736 16874 16953 |
| | 17034 17107 17157 17188 |
| 202: | 10648 10815 10904 10927 11017 11081 11378 11406 11744 11856 11895 12043 12737 12891 13057 13239 |
| | 13837 13892 14082 14124 14197 14234 14662 15102 15949 16255 17286 |
| 203: | 10559 10657 11155 11344 11348 11514 11554 12284 12349 12538 12595 12637 13035 13044 13325 13357 |
| | 13479 13659 13805 14548 14861 15181 15846 15924 15930 15942 16263 16357 16448 16461 16684 16756 17062 |
| | 17363 |
| 204: | 10480 10520 10526 10528 10545 10552 10554 10596 10605 10608 10618 10626 10632 10637 10645 10688 |
| | 10690 10730 10745 10750 10764 10766 10772 10774 10809 10830 10879 10880 10886 10890 10960 10963 10979 |
| | 10985 10991 11001 11004 11025 11026 11031 11065 11080 11106 11127 11130 11152 11154 11157 11172 11176 |
| | 11177 11187 11208 11236 11253 11280 11292 11300 11356 11372 11376 11379 11387 11405 11456 11486 11516 |
| | 11521 11546 11570 11585 11588 11599 11610 11615 11616 11634 11662 11668 11669 11697 11698 11711 11712 |
| | 11720 11731 11758 11760 11770 11797 11812 11829 11836 11855 11864 11876 11890 11907 11937 11938 11945 |
| | 11950 11956 11958 11995 11997 12038 12048 12054 12071 12073 12088 12092 12146 12156 12195 12200 12271 |
| | 12294 12295 12333 12336 12352 12359 12361 12382 12407 12416 12434 12451 12456 12459 12476 12477 12486 |
| | 12497 12513 12531 12547 12554 12557 12558 12562 12588 12589 12611 12622 12655 12657 12658 12667 12681 |
| | 12703 12707 12709 12715 12727 12732 12733 12735 12805 12845 12850 12874 12876 12879 12884 12888 12901 |
| | 12907 12938 12942 12943 12955 12970 12974 12983 12990 13003 13014 13023 13049 13053 13054 13061 13062 |
| | 13064 13074 13090 13111 13119 13155 13174 13236 13238 13241 13242 13261 13264 13277 13284 13295 13313 |
| | 13331 13336 13348 13353 13361 13374 13376 13377 13378 13381 13383 13389 13423 13447 13471 13486 13515 |
| | 13529 13544 13546 13556 13637 13687 13696 13773 13786 13795 13802 13858 13866 13893 13906 13910 13918 |
| | 13923 13927 13942 13946 13956 13965 13994 14005 14051 14071 14073 14083 14097 14101 14117 14121 14138 |
| | 14143 14149 14157 14172 14191 14193 14200 14216 14229 14231 14233 14238 14239 14254 14255 14261 14262 |
| | 14273 14285 14291 14317 14338 14388 14395 14399 14402 14422 14436 14479 14496 14527 14535 14536 14610 |
| | 14612 14618 14633 14640 14651 14653 14672 14675 14685 14693 14699 14704 14714 14719 14736 14740 14750 |
| | 14754 14755 14784 14785 14816 14831 14839 14848 14851 14857 14874 14880 14883 14891 14909 14913 14932 |
| | 14939 14943 14945 14976 14978 15013 15030 15036 15042 15050 15054 15055 15063 15080 15088 15142 15144 |
| | 15148 15176 15197 15203 15214 15219 15230 15236 15255 15261 15307 15324 15328 15331 15333 15337 15340 |
| | 15342 15359 15362 15365 15370 15389 15391 15407 15415 15416 15424 15450 15456 15460 15463 15482 15483 |
| | 15500 15502 15505 15512 15520 15524 15540 15548 15564 15582 15599 15603 15607 15626 15629 15630 15644 |
| | 15656 15683 15693 15711 15720 15731 15734 15738 15761 15801 15803 15818 15834 15839 15847 15851 15871 |
| | 15886 15980 15989 16006 16011 16050 16057 16078 16095 16122 16139 16160 16163 16180 16185 16186 16187 |
| | 16194 16240 16260 16264 16294 16302 16312 16338 16358 16367 16375 16398 16442 16445 16446 16452 16472 |
| | 16483 16484 16499 16503 16510 16538 16558 16562 16568 16608 16617 16627 16631 16640 16646 16647 16662 |
| | 16667 16668 16677 16692 16695 16715 16731 16741 16757 16760 16764 16768 16790 16815 16824 16839 16864 |
| | 16871 16880 16892 16893 16902 16915 16919 16948 16956 16996 16999 17002 17008 17042 17046 17047 17053 |
| | 17072 17115 17119 17131 17144 17145 17150 17185 17220 17234 17247 17269 17270 17292 17295 17296 17298 |
| | 17300 17322 17332 17357 17370 |
| 205: | 10794 10845 11048 11239 11745 11980 12030 12223 12258 12529 12656 12695 12747 12964 13018 13122 |
| | 13338 13339 13413 13499 13826 13891 14176 14449 14524 14677 14820 15060 15077 15309 15888 15962 16268 |
| | 16636 16754 16940 17001 17267 |
| 206: | 10483 10711 10808 10841 10857 11116 11124 11151 11287 11355 11403 11733 11828 11870 11871 11905 |
| | 12089 12105 12335 13204 13708 13924 14188 14272 14437 14540 14600 14650 14659 14759 15069 15394 15516 |
| | 15933 15978 15983 16025 16171 16314 16471 16481 16569 16717 17180 17231 17236 |
| 207: | 10702 11091 11150 12001 12079 12316 12411 12653 13020 13585 13727 14359 14606 14914 14918 14984 |
| | 15576 16725 16941 17097 17129 |
| 208: | 11955 11991 14683 |
| 209: | 10512 10525 10531 10541 10569 10579 10584 10610 10619 10620 10670 10748 10786 10824 10856 10877 |
| | 10915 10920 10930 10954 10977 10983 10994 10999 11000 11016 11019 11021 11043 11049 11082 11087 11105 |
| | 11113 11121 11122 11131 11134 11141 11162 11166 11188 11195 11203 11212 11216 11218 11229 11269 11285 |
| | 11290 11317 11318 11330 11349 11354 11368 11396 11414 11432 11442 11452 11463 11479 11480 11498 11511 |
| | 11525 11545 11565 11578 11579 11591 11593 11594 11598 11602 11613 11637 11638 11746 11757 11779 11780 |
| | 11834 11854 11860 11867 11885 11889 11932 11935 11970 11981 11984 12031 12035 12066 12083 12125 12171 |
| | 12182 12190 12191 12206 12230 12237 12260 12278 12328 12339 12358 12360 12364 12371 12381 12385 12428 |
| | 12435 12438 12442 12462 12466 12493 12506 12508 12517 12522 12586 12594 12624 12627 12634 12638 12642 |
| | 12647 12661 12664 12668 12706 12708 12750 12762 12766 12769 12771 12779 12806 12820 12823 12858 12885 |
| | 12917 12947 12950 12967 12977 13011 13031 13047 13085 13108 13115 13126 13136 13191 13233 13280 13300 |
| | 13316 13329 13333 13335 13343 13400 13428 13432 13454 13457 13578 13607 13650 13651 13673 13684 13690 |
| | 13699 13703 13733 13743 13781 13790 13794 13814 13829 13846 13861 13874 13901 13954 13971 13993 14125 |
| | 14140 14146 14153 14161 14189 14217 14227 14241 14249 14259 14284 14287 14323 14330 14396 14398 14416 |
| | 14470 14471 14487 14490 14508 14519 14541 14550 14552 14571 14573 14576 14626 14631 14638 14689 14700 |
| | 14715 14722 14730 14734 14738 14761 14766 14775 14789 14794 14808 14819 14852 14853 14870 14892 14897 |
| | 14906 14937 14963 14999 15012 15016 15040 15066 15071 15175 15190 15221 15241 15265 15335 15374 15375 |
| | 15381 15438 15453 15465 15484 15485 15526 15577 15581 15595 15596 15600 15613 15645 15647 15648 15653 |
| | 15655 15682 15736 15741 15749 15752 15776 15780 15811 15822 15824 15825 15827 15828 15840 15844 15860 |
| | 15932 15934 15948 15961 15965 15969 15970 15975 16022 16037 16049 16075 16086 16092 16120 16131 16138 |
| | 16143 16152 16166 16169 16174 16184 16193 16197 16208 16214 16245 16298 16311 16437 16444 16450 16491 |
| | 16505 16527 16529 16590 16594 16601 16616 16618 16635 16644 16682 16685 16686 16694 16705 16735 16744 |
| | 16758 16786 16787 16791 16820 16823 16827 16851 16854 16869 16879 16890 16897 16899 16912 16917 16926 |
| | 16930 16939 16946 16985 16987 16988 16990 16994 17009 17011 17028 17031 17096 17125 17153 17155 17161 |
| | 17168 17198 17208 17214 17219 17233 17238 17253 17323 17349 |
| 210: | 10512 10531 10541 10569 10579 10584 10585 10610 10619 10620 10670 10748 10786 10824 10856 10877 |
| | 10915 10920 10930 10954 10977 10983 10994 10999 11000 11016 11019 11021 11043 11082 11087 11105 11113 |
| | 11121 11122 11134 11141 11166 11188 11195 11203 11212 11216 11218 11229 11285 11290 11317 11318 11330 |
| | 11333 11349 11354 11368 11396 11414 11432 11442 11452 11463 11480 11498 11510 11511 11525 11545 11565 |
| | 11578 11579 11591 11593 11594 11598 11602 11613 11638 11746 11779 11780 11834 11854 11860 11867 11885 |
| | 11889 11932 11935 11970 11981 11984 12031 12035 12066 12125 12171 12182 12183 12190 12191 12206 12230 |
| | 12237 12260 12278 12328 12339 12358 12360 12364 12371 12381 12385 12428 12435 12438 12442 12462 12466 |
| | 12493 12506 12508 12517 12522 12524 12586 12594 12624 12627 12630 12634 12638 12647 12661 12664 12706 |
| | 12708 12750 12762 12766 12769 12771 12779 12806 12820 12823 12858 12881 12885 12917 12947 12950 12961 |
| | 12967 12977 13011 13031 13085 13108 13115 13126 13136 13141 13191 13233 13280 13300 13316 13329 13333 |
| | 13343 13400 13428 13432 13454 13457 13578 13607 13650 13651 13673 13684 13690 13699 13703 13733 13743 |
| | 13781 13790 13794 13814 13829 13846 13901 13914 13954 13971 13993 14055 14114 14125 14140 14146 14153 |
| | 14161 14189 14217 14227 14241 14259 14284 14286 14287 14323 14330 14398 14416 14470 14471 14487 14490 |
| | 14508 14519 14541 14550 14571 14573 14576 14626 14631 14638 14689 14700 14715 14722 14730 14734 14738 |
| | 14761 14766 14789 14794 14808 14819 14852 14853 14870 14892 14906 14937 14963 14999 15012 15016 15040 |
| | 15066 15137 15175 15190 15241 15265 15335 15374 15375 15381 15438 15453 15465 15473 15484 15485 15526 |
| | 15577 15581 15595 15596 15600 15613 15645 15647 15648 15653 15655 15682 15736 15741 15743 15749 15752 |
| | 15776 15780 15811 15822 15824 15825 15827 15828 15840 15844 15860 15932 15934 15944 15948 15961 15965 |
| | 15969 15975 16022 16037 16049 16075 16086 16092 16120 16131 16138 16143 16152 16157 16162 16164 16166 |
| | 16169 16174 16184 16193 16197 16208 16214 16245 16273 16298 16311 16437 16444 16450 16491 16505 16527 |
| | 16529 16590 16594 16601 16616 16618 16655 16682 16685 16686 16694 16705 16735 16744 16758 16786 16787 |
| | 16791 16820 16823 16827 16851 16854 16869 16879 16890 16897 16899 16912 16917 16930 16939 16946 16985 |
| | 16987 16988 16990 16994 17009 17011 17028 17031 17096 17125 17153 17155 17161 17168 17198 17208 17219 |
| | 17233 17238 17253 17323 17349 |
| 211: | 10624 11030 11197 11952 12245 12377 12886 13016 13094 13170 13297 13373 13675 13785 14021 14093 |
| | 14502 14745 14846 15162 15620 15798 16137 17166 17189 17245 17273 |
| 213: | 10628 10650 10683 10694 10922 11079 11538 11562 11664 11843 11887 11924 12229 12674 12705 12860 |
| | 13330 13393 13526 13750 13987 14115 14133 14776 14799 14800 15044 15056 15414 15588 15592 15658 16020 |
| | 16614 16755 16799 16938 17014 17112 17143 |
| 215: | 10789 |
| 216: | 10484 10542 10778 11520 11663 11690 12378 12857 13568 14461 15805 15854 15858 16238 16487 16981 |
| 217: | 10828 11215 11394 12100 12204 13012 13758 13937 15262 15870 16740 |
| 218: | 10474 10475 10477 10504 10507 10516 10519 10524 10533 10535 10544 10547 10549 10550 10553 10562 |
| | 10583 10588 10590 10594 10595 10604 10609 10612 10644 10655 10661 10666 10673 10674 10676 10678 10679 |
| | 10685 10693 10701 10706 10709 10710 10712 10715 10719 10720 10723 10725 10729 10732 10734 10751 10765 |
| | 10769 10770 10780 10783 10798 10803 10806 10811 10816 10827 10835 10836 10852 10863 10885 10901 10919 |
| | 10933 10936 10937 10938 10973 10975 10980 10981 11002 11006 11009 11010 11012 11014 11015 11020 11034 |
| | 11035 11061 11062 11085 11089 11092 11096 11100 11110 11120 11132 11133 11142 11145 11148 11149 11153 |
| | 11158 11161 11167 11181 11183 11199 11202 11205 11221 11227 11242 11249 11251 11259 11263 11272 11281 |
| | 11282 11291 11293 11305 11322 11323 11324 11331 11371 11375 11381 11386 11408 11409 11412 11413 11415 |
| | 11425 11431 11440 11446 11449 11465 11468 11469 11487 11488 11490 11497 11505 11506 11532 11537 11548 |
| | 11553 11557 11568 11569 11582 11584 11586 11587 11590 11592 11596 11605 11609 11612 11614 11617 11618 |
| | 11621 11622 11629 11631 11646 11650 11653 11655 11658 11671 11682 11691 11693 11708 11714 11719 11723 |
| | 11737 11738 11739 11740 11743 11763 11768 11776 11778 11785 11830 11837 11839 11842 11847 11850 11851 |
| | 11852 11859 11873 11875 11878 11879 11880 11884 11891 11899 11903 11904 11916 11923 11933 11951 11957 |
| | 11989 11990 12005 12007 12010 12011 12014 12016 12017 12028 12034 12037 12042 12050 12060 12075 12076 |
| | 12078 12087 12109 12112 12113 12115 12116 12121 12124 12139 12151 12162 12179 12217 12227 12231 12241 |
| | 12256 12270 12276 12277 12283 12286 12303 12313 12315 12317 12326 12374 12402 12403 12420 12433 12440 |
| | 12445 12447 12460 12463 12467 12468 12469 12472 12485 12489 12510 12511 12512 12515 12516 12520 12523 |
| | 12530 12533 12536 12537 12539 12541 12542 12543 12550 12552 12555 12564 12568 12576 12577 12578 12581 |
| | 12582 12585 12593 12602 12607 12608 12612 12618 12621 12626 12631 12632 12633 12635 12636 12644 12648 |
| | 12652 12663 12685 12688 12689 12693 12704 12714 12716 12717 12719 12722 12724 12740 12741 12744 12745 |
| | 12748 12767 12768 12770 12789 12793 12804 12809 12813 12817 12828 12854 12869 12872 12880 12919 12932 |
| | 12937 12968 13010 13017 13030 13039 13050 13051 13135 13139 13144 13173 13178 13182 13183 13188 13208 |
| | 13215 13224 13250 13251 13252 13254 13257 13258 13259 13265 13275 13276 13278 13279 13281 13283 13301 |
| | 13356 13362 13363 13364 13365 13384 13397 13401 13407 13409 13411 13414 13422 13426 13429 13431 13434 |
| | 13436 13437 13445 13450 13456 13461 13463 13464 13465 13467 13475 13484 13489 13490 13493 13494 13497 |
| | 13498 13504 13505 13507 13512 13517 13518 13520 13522 13528 13533 13537 13539 13541 13543 13550 13554 |
| | 13555 13559 13561 13562 13564 13567 13572 13573 13584 13588 13590 13592 13593 13596 13598 13599 13605 |
| | 13610 13613 13621 13625 13627 13628 13629 13631 13633 13636 13638 13644 13645 13646 13649 13658 13669 |
| | 13671 13672 13678 13683 13685 13688 13692 13693 13694 13713 13732 13734 13751 13755 13761 13777 13807 |
| | 13820 13838 13855 13856 13863 13867 13878 13880 13882 13887 13889 13890 13896 13899 13903 13907 13908 |
| | 13913 13919 13925 13928 13930 13932 13936 13938 13945 13958 13963 13987 14007 14024 14027 14034 14040 |
| | 14056 14074 14077 14105 14109 14123 14128 14137 14151 14159 14178 14180 14186 14187 14195 14209 14214 |
| | 14235 14244 14250 14263 14264 14265 14270 14289 14292 14299 14303 14306 14307 14308 14309 14310 14314 |
| | 14315 14318 14322 14340 14345 14346 14348 14349 14352 14353 14354 14355 14356 14371 14378 14382 14383 |
| | 14386 14389 14392 14394 14397 14400 14403 14404 14405 14410 14413 14424 14426 14428 14431 14434 14435 |
| | 14438 14464 14465 14466 14468 14473 14474 14475 14480 14482 14485 14495 14497 14498 14499 14500 14503 |
| | 14510 14515 14516 14520 14521 14528 14534 14537 14542 14543 14545 14555 14557 14581 14583 14587 14596 |
| | 14607 14623 14642 14644 14649 14652 14684 14691 14695 14703 14707 14709 14710 14723 14726 14737 14748 |
| | 14772 14774 14783 14786 14810 14814 14817 14826 14847 14850 14862 14863 14868 14879 14881 14888 14901 |
| | 14908 14915 14917 14919 14922 14923 14924 14925 14926 14929 14931 14946 14948 14949 14953 14956 14962 |
| | 14979 15000 15002 15014 15017 15019 15020 15024 15025 15026 15028 15031 15033 15038 15043 15045 15046 |
| | 15047 15049 15051 15057 15061 15082 15087 15089 15094 15100 15104 15108 15109 15113 15114 15115 15116 |
| | 15117 15119 15122 15123 15139 15145 15146 15150 15156 15158 15159 15160 15163 15171 15186 15191 15193 |
| | 15196 15201 15202 15204 15207 15210 15220 15222 15224 15225 15227 15247 15248 15256 15257 15260 15263 |
| | 15266 15268 15271 15279 15285 15288 15296 15302 15303 15310 15314 15343 15346 15349 15352 15353 15355 |
| | 15372 15392 15398 15403 15418 15426 15431 15443 15444 15457 15461 15466 15480 15494 15497 15529 15535 |
| | 15537 15554 15559 15565 15567 15570 15575 15578 15590 15597 15601 15615 15617 15637 15659 15662 15664 |
| | 15665 15667 15680 15689 15698 15730 15739 15742 15762 15764 15782 15786 15820 15826 15843 15875 15876 |
| | 15879 15881 15895 15915 15918 15919 15921 15939 15966 15981 16000 16003 16010 16013 16029 16038 16044 |
| | 16083 16113 16116 16148 16158 16165 16167 16170 16173 16178 16219 16220 16246 16250 16272 16281 16290 |
| | 16292 16295 16307 16315 16317 16318 16320 16323 16328 16332 16355 16360 16364 16366 16368 16370 16372 |
| | 16377 16382 16385 16387 16392 16393 16395 16396 16397 16400 16402 16403 16409 16412 16426 16429 16430 |
| | 16432 16434 16436 16441 16447 16456 16459 16462 16470 16477 16482 16493 16494 16497 16500 16511 16515 |
| | 16516 16517 16519 16523 16535 16541 16545 16553 16576 16577 16582 16586 16589 16603 16605 16606 16607 |
| | 16620 16637 16652 16656 16657 16659 16660 16669 16675 16679 16710 16711 16712 16713 16721 16723 16724 |
| | 16726 16746 16752 16765 16774 16777 16788 16807 16819 16821 16848 16853 16875 16888 16900 16911 16916 |
| | 16923 16951 16952 16957 16978 16982 16995 17019 17021 17026 17049 17063 17075 17081 17110 17114 17120 |
| | 17141 17151 17156 17162 17171 17178 17192 17201 17221 17241 17252 17257 17260 17277 17281 17299 17304 |
| | 17307 17308 17311 17314 17317 17321 17328 17331 17334 17345 17347 17348 17352 17360 17372 |
| 219: | 10473 10489 10494 10537 10539 10551 10568 10570 10573 10614 10631 10634 10689 10747 10756 10767 |
| | 10814 10821 10832 10839 10846 10864 10868 10869 10897 10906 10907 10911 10926 10929 10931 10934 10949 |
| | 10953 10976 10978 10996 11003 11011 11032 11038 11074 11109 11143 11165 11184 11260 11271 11283 11284 |
| | 11298 11347 11360 11362 11422 11429 11434 11445 11450 11451 11459 11502 11528 11533 11534 11535 11543 |
| | 11544 11572 11595 11604 11636 11643 11645 11652 11659 11680 11681 11684 11705 11706 11707 11725 11741 |
| | 11772 11775 11788 11803 11808 11827 11841 11863 11881 11883 11914 11925 11931 11943 11954 11967 12002 |
| | 12025 12067 12095 12111 12122 12167 12168 12172 12236 12263 12275 12280 12282 12306 12332 12337 12347 |
| | 12404 12405 12410 12421 12424 12436 12457 12465 12473 12488 12560 12561 12579 12610 12643 12649 12659 |
| | 12671 12679 12743 12759 12775 12780 12782 12787 12815 12819 12835 12912 12922 12963 12972 12989 12992 |
| | 12993 13005 13007 13021 13022 13036 13045 13058 13066 13076 13088 13093 13107 13137 13142 13153 13165 |
| | 13172 13187 13195 13207 13209 13227 13260 13262 13289 13308 13324 13327 13328 13345 13347 13359 13368 |
| | 13390 13399 13404 13406 13408 13410 13441 13443 13492 13496 13503 13538 13542 13547 13549 13551 13612 |
| | 13617 13635 13640 13643 13710 13719 13720 13730 13737 13745 13747 13754 13770 13784 13801 13812 13819 |
| | 13853 13854 13883 13894 13897 13900 13904 13909 13917 13926 13940 13943 13949 13959 13966 13989 13992 |
| | 13997 14010 14011 14013 14015 14020 14022 14059 14069 14096 14134 14148 14158 14166 14169 14170 14173 |
| | 14181 14182 14184 14210 14213 14218 14219 14226 14245 14246 14253 14296 14301 14313 14373 14432 14440 |
| | 14481 14488 14504 14558 14559 14572 14580 14657 14668 14712 14725 14727 14758 14773 14791 14795 14807 |
| | 14835 14841 14866 14873 14877 14885 14887 14894 14899 14902 14910 14911 14912 14916 14971 14989 14992 |
| | 14993 15035 15110 15126 15188 15272 15290 15295 15334 15367 15376 15399 15406 15412 15430 15441 15468 |
| | 15474 15481 15536 15550 15618 15621 15624 15649 15652 15668 15703 15704 15709 15717 15721 15729 15737 |
| | 15783 15800 15808 15823 15862 15887 15906 15907 15912 15928 15946 15947 15952 15985 16014 16017 16018 |
| | 16059 16068 16073 16076 16084 16088 16101 16110 16147 16177 16188 16201 16206 16215 16216 16217 16233 |
| | 16256 16265 16333 16335 16340 16347 16365 16386 16475 16501 16504 16520 16552 16559 16565 16572 16578 |
| | 16587 16598 16653 16693 16702 16714 16732 16738 16750 16767 16832 16862 16863 16866 16881 16906 16908 |
| | 16910 16924 16929 16942 16945 16949 16950 16958 16963 16965 17006 17015 17017 17036 17038 17052 17077 |
| | 17091 17104 17105 17230 17239 17249 17250 17289 17305 17309 17329 17338 17342 17353 |
| 220: | 10532 10540 10557 10658 10663 10704 10708 10713 10714 10837 10851 11047 11064 11190 11346 11357 |
| | 11383 11574 11666 11833 11886 11909 11998 12012 12013 12086 12131 12144 12225 12379 12452 12514 12616 |
| | 12844 13110 13114 13161 13255 13274 13321 13398 13462 13862 13967 14237 14363 14645 15027 15169 15199 |
| | 15315 15435 15531 15602 15608 15950 16226 16613 16690 16699 16719 17070 17212 17225 17240 17244 17248 |
| | 17276 |
| 221: | 10574 10705 10819 11529 11710 12188 12394 12792 13285 13314 13315 13665 13798 13888 13902 13934 |
| | 14162 14183 14369 14546 14549 14855 15041 15067 15070 15072 15124 15253 15254 15442 15713 15894 16251 |
| | 16348 16401 16651 16920 16962 17176 17339 |
| 222: | 10478 10482 10493 10523 10530 10563 10564 10565 10597 10611 10613 10630 10636 10639 10643 10647 |
| | 10652 10654 10667 10677 10682 10718 10741 10755 10771 10781 10791 10793 10805 10818 10850 10855 10866 |
| | 10867 10894 10905 10910 10943 10955 10992 11005 11036 11037 11040 11041 11046 11059 11060 11063 11076 |
| | 11123 11146 11147 11168 11171 11173 11186 11201 11204 11232 11234 11238 11248 11261 11289 11301 11332 |
| | 11335 11391 11404 11428 11438 11441 11444 11458 11500 11504 11552 11589 11644 11687 11688 11718 11726 |
| | 11730 11748 11767 11791 11804 11823 11826 11831 11861 11862 11874 11898 11902 11920 11976 11983 11987 |
| | 12027 12057 12145 12150 12161 12199 12205 12233 12259 12281 12300 12305 12329 12331 12341 12353 12363 |
| | 12383 12387 12397 12413 12461 12487 12490 12548 12615 12641 12651 12712 12774 12821 12875 12877 12883 |
| | 12890 12926 12940 12957 12969 12995 12999 13029 13056 13086 13087 13092 13097 13099 13147 13197 13210 |
| | 13213 13214 13217 13226 13243 13248 13271 13272 13294 13303 13318 13344 13371 13379 13380 13392 13395 |
| | 13405 13516 13582 13595 13604 13648 13668 13731 13757 13759 13783 13797 13803 13823 13824 13835 13879 |
| | 13920 13964 13976 14106 14112 14131 14206 14242 14247 14321 14335 14372 14393 14406 14419 14439 14454 |
| | 14505 14530 14547 14570 14574 14584 14586 14591 14592 14593 14597 14604 14613 14619 14622 14663 14679 |
| | 14680 14687 14697 14728 14756 14788 14811 14821 14822 14842 14898 14903 14957 14959 14965 14966 14970 |
| | 14996 15001 15022 15029 15078 15092 15136 15165 15194 15232 15321 15336 15338 15341 15345 15348 15356 |
| | 15368 15405 15409 15421 15432 15448 15492 15579 15604 15628 15641 15669 15670 15726 15735 15746 15750 |
| | 15754 15812 15814 15816 15884 15909 15958 16060 16064 16066 16196 16198 16203 16204 16205 16212 16231 |
| | 16236 16259 16271 16341 16349 16419 16476 16485 16547 16556 16566 16592 16597 16629 16663 16665 16678 |
| | 16691 16701 16720 16742 16772 16782 16818 16822 16828 16830 16837 16891 17010 17012 17055 17123 17132 |
| | 17137 17160 17195 17199 17205 17222 17254 17284 17312 17366 |
| 223: | 10478 10482 10493 10523 10530 10563 10564 10565 10597 10611 10613 10630 10636 10639 10643 10647 |
| | 10652 10654 10677 10682 10718 10741 10755 10771 10781 10791 10793 10805 10818 10850 10855 10866 10867 |
| | 10905 10910 10943 10955 10992 11005 11036 11037 11040 11041 11046 11059 11060 11063 11076 11123 11146 |
| | 11147 11168 11171 11173 11186 11201 11204 11232 11234 11238 11248 11261 11289 11301 11332 11335 11404 |
| | 11428 11438 11441 11444 11458 11500 11504 11552 11589 11644 11687 11688 11718 11726 11730 11748 11767 |
| | 11791 11804 11823 11826 11831 11861 11862 11874 11898 11920 11983 11987 12027 12057 12145 12150 12161 |
| | 12199 12205 12233 12259 12281 12300 12305 12329 12331 12341 12353 12363 12383 12387 12413 12461 12487 |
| | 12490 12548 12615 12641 12651 12712 12774 12821 12875 12877 12883 12890 12926 12940 12957 12969 12995 |
| | 12999 13029 13056 13086 13087 13092 13097 13099 13147 13197 13210 13213 13214 13217 13226 13243 13248 |
| | 13271 13272 13294 13303 13318 13344 13371 13380 13392 13395 13405 13516 13582 13595 13604 13648 13668 |
| | 13731 13757 13759 13783 13797 13803 13823 13824 13835 13879 13920 13964 13976 14106 14112 14131 14206 |
| | 14242 14321 14335 14372 14393 14406 14419 14439 14454 14505 14530 14547 14570 14574 14584 14586 14591 |
| | 14592 14593 14597 14604 14613 14619 14622 14663 14680 14697 14728 14756 14788 14811 14821 14822 14842 |
| | 14898 14903 14957 14959 14965 14966 14970 14996 15001 15022 15029 15078 15092 15136 15165 15194 15232 |
| | 15321 15336 15338 15341 15345 15348 15356 15368 15405 15409 15421 15432 15448 15492 15579 15604 15628 |
| | 15641 15669 15670 15726 15735 15746 15750 15754 15812 15814 15816 15884 15909 15958 16060 16064 16066 |
| | 16196 16198 16203 16204 16205 16212 16231 16236 16259 16271 16341 16349 16419 16476 16485 16547 16556 |
| | 16566 16592 16597 16629 16663 16665 16678 16691 16701 16720 16742 16772 16782 16818 16822 16828 16830 |
| | 16837 16891 17010 17012 17055 17123 17132 17137 17195 17199 17205 17222 17254 17284 17312 17366 |
| 224: | 10479 10491 10496 10503 10506 10509 10511 10517 10518 10578 10593 10600 10603 10616 10617 10621 |
| | 10633 10681 10698 10717 10740 10744 10773 10782 10784 10799 10802 10820 10823 10843 10862 10871 10873 |
| | 10875 10882 10891 10939 10942 10948 10974 10990 11008 11052 11057 11068 11070 11072 11073 11084 11099 |
| | 11102 11128 11144 11175 11185 11206 11207 11217 11219 11226 11228 11237 11243 11250 11252 11258 11274 |
| | 11275 11276 11277 11288 11359 11453 11467 11472 11494 11501 11507 11541 11549 11564 11601 11608 11626 |
| | 11633 11657 11660 11661 11670 11695 11709 11732 11735 11749 11756 11783 11786 11787 11796 11799 11801 |
| | 11816 11894 11897 11918 11919 11922 11939 11944 11977 11992 11994 12009 12015 12041 12056 12059 12061 |
| | 12063 12064 12085 12120 12135 12138 12153 12164 12186 12203 12226 12239 12243 12290 12292 12309 12311 |
| | 12343 12367 12369 12390 12399 12414 12419 12425 12431 12453 12475 12498 12518 12532 12535 12546 12566 |
| | 12572 12575 12580 12596 12600 12601 12603 12625 12640 12660 12682 12694 12713 12718 12773 12777 12778 |
| | 12811 12829 12830 12831 12843 12868 12871 12915 12921 12930 12931 12934 12946 12960 12980 12981 12988 |
| | 13019 13033 13043 13046 13069 13101 13104 13118 13152 13206 13228 13263 13298 13299 13317 13342 13350 |
| | 13366 13388 13391 13416 13420 13442 13444 13446 13448 13449 13466 13468 13473 13480 13483 13485 13502 |
| | 13553 13566 13652 13657 13661 13682 13697 13709 13749 13766 13804 13841 13845 13851 13870 13898 13953 |
| | 13957 13975 13977 13981 13985 14001 14052 14062 14066 14068 14092 14110 14136 14147 14154 14156 14174 |
| | 14179 14203 14221 14225 14228 14230 14248 14275 14278 14283 14324 14325 14381 14417 14444 14467 14509 |
| | 14523 14529 14553 14579 14594 14615 14624 14630 14635 14648 14654 14655 14665 14671 14686 14735 14746 |
| | 14801 14812 14823 14824 14836 14844 14872 14878 14890 14893 14905 14907 14942 14987 15006 15009 15015 |
| | 15097 15101 15129 15134 15135 15140 15141 15143 15149 15157 15173 15177 15180 15183 15216 15223 15238 |
| | 15244 15278 15308 15325 15327 15354 15379 15380 15408 15423 15447 15449 15488 15511 15513 15532 15539 |
| | 15563 15573 15585 15593 15594 15614 15631 15633 15654 15674 15679 15701 15702 15706 15710 15724 15748 |
| | 15751 15766 15772 15773 15774 15777 15790 15806 15815 15845 15859 15864 15869 15872 15877 15880 15898 |
| | 15901 15902 15914 15922 15945 15999 16001 16002 16004 16005 16009 16012 16036 16039 16040 16072 16082 |
| | 16108 16112 16144 16145 16146 16168 16175 16176 16181 16189 16209 16227 16237 16257 16262 16266 16284 |
| | 16342 16346 16352 16384 16421 16435 16454 16458 16463 16469 16525 16540 16554 16560 16573 16593 16623 |
| | 16625 16626 16628 16630 16633 16641 16648 16666 16671 16674 16676 16680 16683 16697 16698 16737 16751 |
| | 16753 16766 16770 16771 16785 16794 16814 16816 16834 16850 16856 16858 16861 16877 16895 16896 16960 |
| | 16972 16975 16976 16977 16989 16992 17023 17040 17068 17095 17102 17226 17246 17251 17278 17279 17302 |
| | 17303 17318 17354 17373 17374 |
| 225: | 10476 10486 10488 10500 10502 10508 10513 10527 10566 10567 10571 10572 10598 10599 10656 10659 |
| | 10660 10662 10664 10665 10669 10687 10716 10736 10749 10753 10761 10775 10776 10792 10797 10801 10804 |
| | 10822 10834 10858 10865 10876 10878 10881 10888 10896 10900 10917 10918 10932 10947 10952 10959 10968 |
| | 10988 11013 11042 11051 11056 11075 11077 11086 11088 11093 11094 11095 11117 11136 11140 11156 11160 |
| | 11178 11182 11193 11194 11214 11225 11231 11233 11235 11264 11265 11267 11296 11297 11310 11321 11329 |
| | 11343 11350 11352 11353 11384 11400 11402 11410 11411 11419 11421 11424 11443 11448 11462 11470 11478 |
| | 11519 11536 11540 11567 11571 11573 11597 11611 11635 11639 11640 11641 11642 11649 11651 11654 11667 |
| | 11672 11673 11676 11677 11678 11679 11700 11717 11722 11727 11729 11755 11769 11771 11773 11774 11789 |
| | 11800 11814 11818 11822 11825 11840 11866 11877 11896 11901 11908 11917 11941 11960 11965 11966 11968 |
| | 11971 11972 11988 11996 12023 12046 12053 12055 12058 12090 12091 12128 12143 12163 12166 12169 12211 |
| | 12216 12228 12234 12235 12248 12255 12268 12296 12308 12321 12325 12340 12362 12398 12400 12412 12423 |
| | 12426 12427 12432 12437 12439 12441 12454 12464 12478 12480 12496 12507 12544 12545 12553 12563 12606 |
| | 12620 12646 12650 12662 12666 12676 12678 12699 12702 12720 12723 12746 12751 12765 12772 12786 12790 |
| | 12794 12796 12800 12803 12816 12824 12825 12827 12856 12870 12887 12896 12897 12900 12906 12929 12941 |
| | 12998 13009 13048 13067 13071 13073 13084 13103 13106 13131 13134 13154 13171 13180 13181 13185 13201 |
| | 13205 13247 13253 13287 13290 13291 13305 13312 13322 13334 13337 13340 13349 13351 13360 13396 13418 |
| | 13419 13421 13438 13440 13459 13460 13478 13482 13501 13508 13509 13514 13536 13545 13565 13580 13624 |
| | 13674 13698 13706 13716 13717 13725 13753 13778 13800 13817 13825 13833 13834 13836 13857 13868 13876 |
| | 13884 13905 13912 13933 13960 13968 13983 13986 13991 13999 14014 14016 14019 14031 14037 14041 14046 |
| | 14064 14075 14080 14088 14091 14094 14098 14099 14108 14129 14132 14145 14152 14167 14175 14190 14204 |
| | 14205 14207 14215 14223 14232 14243 14260 14271 14277 14282 14316 14320 14329 14343 14357 14361 14364 |
| | 14380 14409 14415 14425 14429 14455 14472 14484 14492 14512 14514 14531 14533 14538 14544 14561 14567 |
| | 14568 14598 14599 14611 14614 14616 14636 14637 14639 14646 14656 14661 14674 14676 14678 14681 14692 |
| | 14698 14716 14718 14721 14724 14729 14742 14751 14753 14757 14762 14764 14805 14806 14815 14830 14837 |
| | 14843 14849 14856 14896 14935 14944 14947 14950 14961 14969 14982 15005 15008 15018 15021 15023 15034 |
| | 15048 15059 15065 15076 15079 15081 15090 15091 15118 15128 15133 15147 15174 15185 15189 15205 15209 |
| | 15211 15212 15215 15217 15234 15235 15250 15251 15252 15326 15332 15347 15373 15377 15388 15395 15397 |
| | 15417 15420 15428 15434 15437 15439 15446 15455 15489 15490 15491 15496 15499 15504 15510 15515 15518 |
| | 15521 15528 15561 15569 15571 15574 15583 15605 15623 15625 15635 15638 15681 15699 15708 15715 15792 |
| | 15793 15797 15817 15821 15838 15841 15850 15856 15861 15873 15882 15883 15889 15891 15900 15931 15937 |
| | 15938 15963 15964 15971 15974 15977 16007 16019 16024 16043 16081 16109 16118 16119 16191 16224 16229 |
| | 16232 16234 16235 16261 16287 16291 16297 16299 16309 16343 16344 16353 16390 16404 16411 16414 16486 |
| | 16507 16557 16563 16564 16585 16599 16602 16612 16624 16643 16649 16658 16670 16672 16673 16681 16696 |
| | 16709 16716 16729 16730 16734 16739 16748 16759 16763 16776 16789 16802 16809 16825 16842 16859 16860 |
| | 16901 16903 16909 16961 16974 16983 16991 16998 17000 17003 17005 17007 17018 17022 17027 17030 17066 |
| | 17067 17078 17092 17100 17101 17113 17128 17149 17175 17179 17182 17191 17194 17203 17204 17207 17215 |
| | 17232 17262 17265 17268 17271 17280 17283 17285 17306 17313 17376 |
| 226: | 10662 10775 10801 10804 10952 11117 11193 11214 11231 11649 11651 11676 11678 11679 11814 12163 |
| | 12228 12400 12800 13106 13171 13340 13396 13508 13717 13983 14014 14099 14132 14190 14277 14380 14472 |
| | 14753 14837 14843 15189 15420 15490 15605 15635 16624 16649 16730 16763 16909 17027 17113 17191 17207 |
| | 17280 |
| 227: | 10950 11017 11107 11198 11210 11304 11517 11566 11759 11777 11846 11900 11929 11964 12165 12176 |
| | 12257 12351 12629 12826 12987 13113 13472 13715 13771 13865 13921 13935 14082 14171 14280 14288 14311 |
| | 14643 14769 14927 15245 15360 15440 15684 15732 16140 16150 16207 16275 16496 16611 16650 16979 |
| 228: | 10838 10889 11126 11393 11561 11858 12024 13367 13608 13609 13656 13827 13864 13955 14127 14211 |
| | 14628 14629 14768 15084 15153 15317 16369 16610 16775 16806 16852 17202 |
| 230: | 10505 10556 10635 10672 10759 10860 10946 10986 10998 11200 11241 11316 11328 11351 11407 11476 |
| | 11477 11482 11483 11555 11558 11560 11575 11577 11580 11581 11628 11782 11795 11821 11849 11865 11949 |
| | 12072 12099 12102 12106 12108 12126 12127 12129 12132 12133 12134 12137 12142 12149 12178 12193 12320 |
| | 12324 12344 12391 12422 12443 12483 12484 12528 12534 12540 12551 12599 12619 12692 12839 12878 13138 |
| | 13151 13245 13246 13273 13288 13307 13311 13320 13474 13571 13626 13632 13634 13639 13681 13686 13689 |
| | 13769 13831 13848 13871 13911 13929 14026 14044 14087 14412 14441 14442 14493 14517 14518 14588 14641 |
| | 14658 14706 14708 14731 14828 14834 14882 14889 14936 15062 15064 15228 15281 15283 15284 15289 15291 |
| | 15312 15316 15319 15322 15445 15553 15598 15673 15695 15778 15830 15908 15913 15940 16046 16052 16074 |
| | 16080 16085 16183 16218 16296 16316 16337 16423 16722 17135 17163 17343 17356 17375 |
| 231: | 10703 10903 11022 11090 11320 11551 11930 11982 11993 12004 12298 12417 12758 12851 12952 13270 |
| | 13332 13387 14025 14050 14089 14305 14627 14941 14960 15075 15184 15454 15517 15627 15643 15671 15733 |
| | 15760 15767 15768 15829 15916 15994 16179 16308 16468 16621 16708 16927 17211 17316 |
| 232: | 10543 10800 11044 14341 14390 14733 15514 15611 16225 17148 |
| 233: | 10592 10722 10908 10940 10944 11055 11125 11374 11481 11539 11892 12026 12448 12491 12598 12680 |
| | 12799 12834 12838 13098 13304 13531 13691 13886 14023 14625 14760 14802 14838 14933 15103 15164 15192 |
| | 15195 15853 16041 16077 16091 16107 16728 16762 17369 |
| 234: | 10763 10970 11270 11299 11382 11620 11648 11868 12202 12691 12701 12841 12853 12986 13078 13430 |
| | 13611 13832 13961 14045 14198 14577 14900 14997 15300 15402 15546 15874 15903 15910 15920 16035 16151 |
| | 16410 16596 16638 16689 16769 |
| 235: | 10495 10497 10499 10534 10546 10558 10576 10577 10587 10646 10651 10671 10675 10742 10743 10746 |
| | 10785 10788 10807 10817 10826 10833 10844 10849 10859 10861 10870 10874 10884 10887 10895 10898 10902 |
| | 10913 10923 10928 10935 10941 10951 10972 11007 11028 11067 11071 11078 11103 11104 11112 11115 11129 |
| | 11139 11174 11180 11189 11191 11192 11196 11209 11213 11224 11230 11247 11256 11257 11262 11302 11307 |
| | 11313 11319 11334 11370 11373 11437 11447 11473 11474 11489 11513 11527 11550 11556 11576 11600 11619 |
| | 11623 11627 11632 11686 11696 11702 11704 11716 11721 11728 11734 11751 11752 11761 11790 11793 11805 |
| | 11811 11813 11820 11824 11835 11845 11848 11872 11882 11921 11927 11928 11934 11942 11947 11948 11961 |
| | 11962 11963 11974 11978 11979 12000 12003 12021 12045 12069 12117 12119 12170 12194 12197 12210 12214 |
| | 12232 12246 12250 12254 12262 12273 12274 12293 12307 12318 12330 12350 12354 12356 12365 12372 12389 |
| | 12396 12430 12446 12471 12494 12495 12503 12505 12519 12525 12556 12567 12571 12573 12574 12587 12597 |
| | 12614 12617 12623 12639 12665 12687 12700 12721 12734 12749 12752 12755 12785 12798 12807 12814 12847 |
| | 12849 12852 12863 12905 12913 12959 12962 12966 12973 12994 13006 13025 13026 13060 13081 13095 13129 |
| | 13130 13167 13184 13200 13225 13231 13256 13267 13269 13293 13309 13310 13319 13326 13341 13352 13415 |
| | 13417 13427 13433 13469 13487 13525 13527 13530 13532 13535 13569 13574 13587 13589 13594 13615 13620 |
| | 13623 13655 13705 13714 13740 13741 13742 13764 13765 13768 13774 13775 13776 13779 13788 13792 13811 |
| | 13815 13828 13830 13852 13860 13873 13875 13877 13881 13885 13948 13982 13990 13995 14004 14006 14009 |
| | 14028 14029 14033 14048 14049 14053 14072 14084 14086 14095 14116 14122 14130 14144 14160 14164 14177 |
| | 14196 14252 14257 14267 14269 14279 14281 14304 14350 14360 14385 14433 14445 14446 14451 14477 14494 |
| | 14501 14511 14513 14525 14556 14585 14595 14605 14620 14666 14669 14673 14702 14767 14787 14798 14804 |
| | 14818 14833 14864 14895 14930 14940 14951 14954 14972 14973 14977 14985 14994 15004 15010 15011 15073 |
| | 15074 15085 15086 15099 15106 15127 15131 15151 15152 15170 15187 15218 15249 15259 15270 15273 15277 |
| | 15320 15329 15358 15369 15470 15477 15486 15487 15506 15507 15566 15610 15640 15650 15660 15675 15688 |
| | 15691 15697 15707 15714 15719 15747 15753 15755 15756 15757 15758 15759 15770 15837 15857 15885 15905 |
| | 15925 15960 15968 15982 15997 15998 16008 16045 16065 16089 16114 16117 16136 16155 16210 16228 16239 |
| | 16244 16249 16274 16276 16304 16336 16351 16376 16383 16391 16399 16406 16407 16413 16416 16418 16440 |
| | 16449 16478 16489 16506 16513 16522 16531 16555 16581 16634 16664 16703 16704 16743 16817 16846 16887 |
| | 16904 16918 16922 16925 16928 16932 16954 16955 16967 16970 16973 16984 16993 16997 17004 17020 17024 |
| | 17025 17032 17044 17048 17099 17117 17118 17122 17127 17147 17158 17164 17173 17200 17217 17235 17263 |
| | 17274 17301 17315 17319 17325 17341 17344 |
| 236: | 11742 12406 |
| 237: | 11223 11832 12406 12559 12911 16502 |
| 238: | 10840 11211 11713 11815 11817 11819 11893 12181 12185 12192 12388 12776 13286 13453 13667 13772 |
| | 14047 14090 14150 14532 14562 14566 14603 14647 14664 14743 14763 14779 14860 14867 14934 15007 15032 |
| | 15364 15586 15831 16134 16195 16270 16286 16301 16706 16707 16749 16784 16886 16986 17033 17035 17039 |
| | 17060 17121 17264 17293 17350 17362 |
| 239: | 10563 10611 10639 10643 10647 10677 10793 10955 11040 11046 11147 11238 11718 11898 12150 12281 |
| | 12329 12341 12641 12651 12774 12883 12926 12999 13097 13099 13371 13604 13835 13964 14106 14112 14131 |
| | 14372 14547 14597 14613 14756 14970 15194 15232 15341 15345 15628 15669 16064 16198 16203 16205 16271 |
| | 16691 16837 17012 17055 17132 17284 |
| 240: | 10529 10602 10606 10629 10707 10728 10752 10760 10779 10825 10831 10853 10872 10893 11027 11108 |
| | 11159 11245 11315 11377 11420 11427 11454 11499 11559 11583 11689 11701 11794 11853 11857 11946 11973 |
| | 12020 12081 12201 12253 12570 12882 12893 12910 12945 12948 12975 12976 12978 12979 12982 12996 12997 |
| | 13000 13001 13002 13004 13008 13038 13040 13041 13042 13302 13323 13439 13603 13680 13736 13739 13744 |
| | 13746 13796 13840 13939 13984 14100 14168 14236 14326 14334 14401 14522 14551 14582 14601 14602 14608 |
| | 14632 14634 14688 14797 14809 14845 14886 14920 15112 15130 15292 15293 15339 15344 15351 15382 15383 |
| | 15384 15385 15387 15401 15509 15519 15522 15538 15545 15549 15551 15552 15555 15580 15587 15589 15591 |
| | 15609 15634 15636 15663 15666 15687 15690 15692 15718 15722 15745 15763 15765 15787 15796 15799 15804 |
| | 15842 15867 15893 15926 15929 16042 16079 16130 16133 16135 16142 16159 16172 16221 16278 16326 16331 |
| | 16334 16371 16374 16420 16439 16473 16526 16548 16549 16550 16551 16584 16615 16747 16934 17013 17059 |
| | 17079 17080 17083 |
| 241: | 10582 10601 10813 10945 11279 11547 11675 11736 12355 12482 12590 12609 12909 12939 13034 13102 |
| | 13105 13121 13306 13679 13718 13842 13922 13962 14000 14212 14319 14367 14526 14564 14590 14749 14854 |
| | 14871 14938 14998 15098 15111 15413 15523 15661 15771 15775 15935 16213 16580 16609 17090 17116 17139 |
| | 17237 17275 17288 17310 17355 |
| 242: | 10481 10510 10522 10548 10555 10560 10627 10640 10649 10653 10680 10686 10692 10721 10724 10727 |
| | 10731 10733 10735 10737 10739 10757 10758 10812 10899 10958 10962 10967 10969 10971 10987 10989 10993 |
| | 10995 10997 11023 11050 11111 11244 11255 11266 11306 11308 11309 11312 11314 11336 11337 11338 11339 |
| | 11340 11341 11342 11345 11361 11363 11364 11365 11366 11367 11369 11385 11389 11390 11392 11395 11397 |
| | 11398 11399 11401 11416 11417 11423 11426 11430 11435 11439 11455 11457 11461 11464 11466 11491 11492 |
| | 11493 11495 11496 11512 11515 11518 11522 11524 11526 11530 11563 11603 11625 11656 11685 11750 11766 |
| | 11913 11985 11986 12006 12019 12022 12036 12039 12040 12044 12049 12052 12074 12077 12080 12082 12084 |
| | 12096 12097 12098 12101 12103 12104 12107 12110 12114 12118 12136 12140 12141 12147 12148 12152 12154 |
| | 12157 12158 12159 12160 12177 12180 12184 12187 12189 12198 12209 12212 12215 12218 12219 12221 12224 |
| | 12238 12240 12242 12244 12247 12249 12251 12252 12261 12264 12265 12267 12269 12272 12285 12288 12291 |
| | 12297 12299 12301 12304 12312 12319 12345 12346 12348 12368 12370 12373 12376 12380 12429 12470 12474 |
| | 12501 12502 12504 12509 12605 12613 12628 12669 12670 12672 12675 12696 12697 12698 12710 12711 12725 |
| | 12726 12728 12730 12731 12739 12753 12756 12763 12781 12784 12795 12797 12801 12802 12808 12810 12818 |
| | 12837 12840 12842 12846 12848 12861 12862 12864 12865 12867 12892 12894 12895 12898 12899 12902 12920 |
| | 12924 12925 12927 12928 12944 12949 12951 12953 12956 12958 12984 12985 13024 13028 13032 13052 13055 |
| | 13075 13080 13082 13091 13096 13109 13112 13116 13117 13120 13123 13124 13125 13127 13128 13143 13146 |
| | 13148 13150 13156 13159 13162 13163 13164 13175 13176 13179 13186 13189 13190 13193 13196 13198 13199 |
| | 13202 13212 13219 13222 13223 13229 13230 13232 13234 13235 13237 13249 13346 13403 13425 13452 13455 |
| | 13458 13481 13488 13491 13500 13506 13519 13521 13524 13534 13540 13548 13557 13558 13560 13563 13570 |
| | 13576 13577 13579 13581 13583 13586 13591 13597 13601 13606 13614 13616 13619 13622 13630 13641 13642 |
| | 13647 13654 13660 13663 13666 13670 13676 13677 13700 13704 13707 13711 13712 13722 13723 13724 13726 |
| | 13728 13729 13735 13738 13748 13752 13756 13760 13762 13780 13782 13787 13789 13791 13810 13813 13816 |
| | 13818 13821 13839 13844 13847 13849 13850 13869 13895 13916 13941 13944 13947 13950 13951 13969 13972 |
| | 13974 13978 13979 13980 13996 13998 14002 14003 14008 14012 14030 14032 14035 14036 14039 14043 14054 |
| | 14058 14061 14063 14065 14078 14163 14251 14266 14268 14274 14293 14295 14328 14331 14333 14336 14337 |
| | 14339 14342 14365 14370 14374 14376 14377 14379 14384 14387 14391 14408 14411 14414 14418 14420 14421 |
| | 14423 14427 14443 14447 14448 14450 14452 14457 14459 14460 14462 14463 14469 14476 14483 14489 14491 |
| | 14660 14711 14717 14741 14752 14765 14770 14771 14781 14827 14869 14981 14983 14986 14990 14991 15058 |
| | 15239 15264 15267 15269 15276 15280 15282 15286 15298 15299 15304 15305 15311 15313 15350 15357 15361 |
| | 15366 15371 15386 15390 15393 15396 15400 15410 15419 15422 15427 15429 15433 15451 15452 15458 15459 |
| | 15462 15479 15493 15495 15498 15501 15503 15508 15525 15527 15530 15533 15534 15541 15542 15543 15544 |
| | 15557 15560 15562 15568 15572 15612 15616 15619 15632 15639 15642 15657 15672 15685 15696 15700 15705 |
| | 15727 15728 15779 15784 15789 15791 15794 15802 15810 15813 15865 15868 15892 15896 15899 15923 15953 |
| | 15955 15957 15959 15984 15986 15987 15988 15990 15991 15992 15993 15995 15996 16021 16023 16026 16027 |
| | 16028 16030 16031 16032 16033 16034 16048 16051 16053 16054 16055 16056 16058 16061 16062 16063 16067 |
| | 16069 16090 16093 16094 16096 16097 16098 16099 16100 16102 16103 16104 16105 16106 16111 16121 16123 |
| | 16125 16127 16128 16129 16182 16241 16242 16243 16247 16248 16252 16253 16254 16258 16267 16269 16277 |
| | 16279 16282 16283 16285 16288 16293 16303 16305 16306 16310 16313 16319 16322 16324 16325 16329 16330 |
| | 16345 16350 16356 16359 16361 16362 16363 16373 16379 16381 16388 16389 16394 16415 16417 16422 16424 |
| | 16427 16428 16431 16433 16438 16443 16453 16455 16457 16460 16464 16466 16488 16490 16495 16498 16509 |
| | 16512 16514 16518 16528 16530 16533 16536 16537 16539 16542 16543 16546 16561 16570 16571 16579 16583 |
| | 16588 16727 16733 16778 16779 16780 16781 16783 16792 16793 16795 16797 16798 16801 16803 16804 16805 |
| | 16808 16811 16812 16813 16826 16829 16831 16833 16835 16836 16838 16840 16841 16843 16844 16867 16868 |
| | 16870 16872 16873 16876 16878 16882 16883 16884 16885 16905 16907 16913 16931 16933 16935 16936 16937 |
| | 16943 16964 16966 16968 16969 16971 17029 17037 17054 17056 17057 17058 17064 17065 17071 17073 17076 |
| | 17082 17085 17087 17088 17089 17094 17098 17103 17106 17109 17111 17124 17126 17130 17133 17134 17138 |
| | 17140 17142 17146 17152 17165 17167 17170 17172 17174 17177 17181 17183 17186 17187 17190 17193 17196 |
| | 17206 17209 17210 17213 17216 17218 17223 17224 17227 17228 17255 17256 17258 17259 17266 17272 17287 |
| | 17290 17291 17297 17320 17324 17326 17330 17333 17335 17336 17337 17340 17367 |

Recombinant DNA constructs are prepared using the DNA encoding each of the identified homologs and the constructs are used to prepare multiple events of transgenic corn, soybean, canola and cotton plants as illustrated in Examples 2-5. Plants are regenerated from the transformed plant cells and used to produce progeny plants and seed that are screened for enhanced water use efficiency, enhanced cold tolerance, increased yield, enhanced nitrogen use efficiency, enhanced seed protein and enhanced seed oil. From each group of multiple events of transgenic plants with a specific recombinant DNA for a homolog the event that produces the greatest enhancement in yield, water use efficiency, nitrogen use efficiency, enhanced cold tolerance, enhanced seed protein and enhanced seed oil is identified and progeny seed is selected for commercial development.

### Example 7

### Consensus Sequence

This example illustrates the identification of consensus amino acid sequence for the proteins and homologs encoded by DNA that is used to prepare the transgenic seed and plants of this invention having enhanced agronomic traits.

ClustalW program was selected for multiple sequence alignments of the amino acid sequence of SEQ ID NO: 122, 133, 136, 138, 141, 143, 153, 155, 173-174, 182, 197, 203, 206, 208, 215, 217, 229, 234, 236-237, 240 and their homologs. Three major factors affecting the sequence alignments dramatically are (1) protein weight matrices; (2) gap open penalty; (3) gap extension penalty. Protein weight matrices available for ClustalW program include Blosum, Pam and Gonnet series. Those parameters with gap open penalty and gap extension penalty were extensively tested. On the basis of the test results, Blosum weight matrix, gap open penalty of 10 and gap extension penalty of 1 were chosen for multiple sequence alignment.

The consensus amino acid sequence can be used to identify DNA corresponding to the full scope of this invention that is useful in providing transgenic plants, for example corn and soybean plants with enhanced agronomic traits, for example improved nitrogen use efficiency, improved yield, improved water use efficiency and/or improved growth under cold stress, due to the expression in the plants of DNA suppressing a protein with amino acid sequence identical to the consensus amino acid sequence.

The SEQ ID NOs for the identified consensus sequences are reported in table 8 below and the full consensus sequences are provided in the attached sequence listing.

TABLE 8

| | | Consensus |
|---|---|---|
| Gene ID | PEP SEQ ID NO | SEQ ID NO |
| Mnom000132-Mnom000134 | 122 | 17381 |
| Mnom000451 | 133 | 17382 |
| Mnom000481 | 136 | 17383 |
| Mnom000486 | 138 | 17384 |
| Mnom000494 | 141 | 17385 |
| Mnom000497 | 143 | 17386 |
| Mnom000636 | 153 | 17387 |
| Mnom000640 | 155 | 17388 |
| Mnom000766-Mnom000767 | 173 | 17389 |
| Mnom000822-Mnom000823 | 174 | 17390 |
| Mnom000986 | 182 | 17391 |
| PHE0002862 | 197 | 17392 |
| PHE0004853 | 203 | 17393 |
| PHE0006378 | 206 | 17394 |
| PHE0006496 | 208 | 17395 |
| PHE0008407 | 215 | 17396 |
| PHE0008541 | 217 | 17397 |
| PHE0011028 | 229 | 17398 |
| PHE0013205 | 234 | 17399 |
| PHE0013712 | 236 | 17400 |
| PHE0013713 | 237 | 17401 |
| TRG0000291 | 240 | 17402 |

### Example 9

### Identification of Amino Acid Domain by Pfam Analysis

This example illustrates the identification of domain and domain module by Pfam analysis.

The amino acid sequence of the expressed proteins that are shown to be associated with an enhanced trait were analyzed for Pfam protein family against the current Pfam collection of multiple sequence alignments and hidden Markov models using the HMMER software in the appended computer listing. The Pfam protein domains and modules for the proteins of SEQ ID NOs: 123-132, 134-135, 137, 139-140, 142, 145-152, 154, 156-172, 175-181, 183-196, 198-202, 204-205, 207, 209-211, 213, 216, 218-228, 230-233, 235, 238-239, and 241-242 are shown in Tables 9, 10 and 11. The Hidden Markov model databases for the identified patent families are also in the appended computer listing allowing identification of other homologous proteins and their cognate encoding DNA to enable the full breadth of the invention for a person of ordinary skill in the art. Certain proteins are identified by a single Pfam domain and others by multiple Pfam domains.

TABLE 9
Pfam annotation

| PEP SEQ ID NO | Gene ID | Pfam domain name | Begin | Stop | Score | E-value |
|---|---|---|---|---|---|---|
| 123 | Mnom000202 | Copine | 116 | 264 | 331.7 | 1.50E−96 |
| 124 | Mnom000215-Mnom000216 | CBFB_NFYA | 97 | 153 | 143.1 | 8.50E−40 |
| 125 | Mnom000219-Mnom000220 | CBFD_NFYB_HMF | 25 | 90 | 124.7 | 3.10E−34 |
| 126 | Mnom000256-Mnom000257 | Glu_synthase | 115 | 494 | 683.3 | 2.00E−202 |
| 127 | Mnom000260-Mnom000261 | HEAT | 183 | 219 | 22.6 | 0.0016 |
| 127 | Mnom000260-Mnom000261 | HEAT | 716 | 753 | 17.5 | 0.057 |
| 127 | Mnom000260-Mnom000261 | FAT | 1435 | 1801 | 483.5 | 2.90E−142 |
| 127 | Mnom000260-Mnom000261 | Rapamycin_bind | 1908 | 2007 | 232.8 | 8.80E−67 |
| 127 | Mnom000260-Mnom000261 | PI3_PI4_kinase | 2074 | 2324 | 370.7 | 2.70E−108 |
| 127 | Mnom000260-Mnom000261 | FATC | 2432 | 2464 | 65.9 | 1.60E−16 |
| 128 | Mnom000262 | F-box | 29 | 76 | 36.2 | 1.30E−07 |
| 129 | Mnom000324 | AP2 | 136 | 188 | 81.9 | 2.30E−21 |
| 130 | Mnom000372 | SAP | 34 | 68 | 45.6 | 1.90E−10 |
| 130 | Mnom000372 | zf-MIZ | 357 | 406 | 111.3 | 3.20E−30 |
| 131 | Mnom000412-Mnom000413 | 14-3-3 | 4 | 219 | 277.4 | 3.20E−80 |
| 132 | Mnom000444 | GATase_2 | 39 | 409 | 772.4 | 3.20E−229 |
| 132 | Mnom000444 | Glu_syn_central | 483 | 784 | 680.2 | 1.80E−201 |
| 132 | Mnom000444 | Glu_synthase | 841 | 1226 | 867.4 | 8.10E−258 |
| 132 | Mnom000444 | GXGXG | 1304 | 1499 | 387.2 | 2.90E−113 |
| 134 | Mnom000471 | Glyco_transf_20 | 16 | 482 | 1010.3 | 7.40E−301 |
| 134 | Mnom000471 | Trehalose_PPase | 572 | 819 | 382.5 | 7.50E−112 |
| 135 | Mnom000480 | Response_reg | 16 | 128 | 26.4 | 5.90E−06 |
| 137 | Mnom000485 | FAR1 | 67 | 160 | 131.4 | 2.90E−36 |
| 137 | Mnom000485 | FART | 204 | 298 | 126.8 | 6.90E−35 |
| 139 | Mnom000490 | PTR2 | 1 | 257 | 46.1 | 1.30E−10 |
| 140 | Mnom000491 | PPR | 151 | 185 | 14.4 | 0.18 |
| 140 | Mnom000491 | PPR | 186 | 219 | 11.9 | 0.35 |
| 140 | Mnom000491 | PPR | 220 | 254 | 5.4 | 2 |
| 140 | Mnom000491 | PPR | 258 | 292 | 18.4 | 0.029 |
| 140 | Mnom000491 | PPR | 293 | 327 | 25.4 | 0.00024 |
| 140 | Mnom000491 | PPR | 328 | 362 | 26.1 | 0.00014 |
| 140 | Mnom000491 | PPR | 363 | 397 | 32 | 2.40E−06 |
| 140 | Mnom000491 | PPR | 398 | 432 | 12.1 | 0.33 |
| 140 | Mnom000491 | PPR | 434 | 468 | 23.7 | 0.00076 |
| 140 | Mnom000491 | PPR | 469 | 503 | 29.4 | 1.50E−05 |
| 140 | Mnom000491 | PPR | 534 | 568 | 34.5 | 4.20E−07 |
| 142 | Mnom000496 | efhand | 109 | 137 | 23.6 | 0.00082 |
| 145 | Mnom000517 | 14-3-3 | 8 | 225 | 391.1 | 1.90E−114 |
| 146 | Mnom000554 | DUF761 | 9 | 329 | 302.9 | 6.80E−88 |
| 147 | Mnom000570 | SAP | 11 | 45 | 49.3 | 1.50E−11 |
| 147 | Mnom000570 | PHD | 114 | 168 | 35.4 | 2.20E−07 |
| 147 | Mnom000570 | zf-MIZ | 359 | 408 | 88.1 | 3.10E−23 |
| 148 | Mnom000580 | HSP20 | 133 | 230 | 144.1 | 4.20E−40 |
| 149 | Mnom000616-Mnom000617 | Fer2 | 19 | 88 | 47.5 | 5.30E−11 |
| 149 | Mnom000616-Mnom000617 | Fer2_2 | 98 | 173 | 127.1 | 5.70E−35 |
| 149 | Mnom000616-Mnom000617 | FAD_binding_5 | 260 | 440 | 273.4 | 5.10E−79 |
| 149 | Mnom000616-Mnom000617 | CO_deh_flav_C | 447 | 558 | 151.7 | 2.20E−42 |
| 149 | Mnom000616-Mnom000617 | Ald_Xan_dh_C | 612 | 719 | 168.1 | 2.60E−47 |
| 149 | Mnom000616-Mnom000617 | Ald_Xan_dh_C2 | 727 | 1272 | 756.1 | 2.50E−224 |
| 150 | Mnom000618-Mnom000619 | Fer2 | 11 | 80 | 42.5 | 1.70E−09 |
| 150 | Mnom000618-Mnom000619 | Fer2_2 | 90 | 165 | 126.5 | 8.50E−35 |
| 150 | Mnom000618-Mnom000619 | FAD_binding_5 | 252 | 432 | 271.6 | 1.80E−78 |
| 150 | Mnom000618-Mnom000619 | CO_deh_flav_C | 439 | 550 | 145.5 | 1.60E−40 |
| 150 | Mnom000618-Mnom000619 | Ald_Xan_dh_C | 604 | 711 | 159.3 | 1.20E−44 |
| 150 | Mnom000618-Mnom000619 | Ald_Xan_dh_C2 | 719 | 1264 | 746.6 | 1.90E−221 |
| 151 | Mnom000626-Mnom000627 | 2OG-FeII_Oxy | 180 | 280 | 121.3 | 3.10E−33 |
| 152 | Mnom000628 | 2OG-Fell_Oxy | 180 | 280 | 121.3 | 3.10E−33 |
| 154 | Mnom000637 | B3 | 312 | 417 | 118.5 | 2.30E−32 |
| 154 | Mnom000637 | Auxin_resp | 439 | 524 | 160.7 | 4.30E−45 |
| 154 | Mnom000637 | AUX_IAA | 681 | 826 | −74.9 | 0.00048 |
| 156 | Mnom000641 | XRN_N | 1 | 256 | 589.7 | 3.10E−174 |
| 156 | Mnom000641 | zf-CCHC | 263 | 280 | 28.9 | 8.30E−06 |
| 157 | Mnom000643 | PALP | 36 | 329 | 341 | 2.30E−99 |
| 158 | Mnom000645 | DAO | 164 | 518 | −26.1 | 0.00058 |
| 158 | Mnom000645 | GMC_oxred_N | 210 | 434 | 358.8 | 1.00E−104 |
| 159 | Mnom000647 | Peptidase_S10 | 42 | 455 | 562.2 | 6.00E−166 |
| 160 | Mnom000682 | Glycos_transf_1 | 296 | 459 | 121.8 | 2.20E−33 |
| 161 | Mnom000690 | Redoxin | 76 | 232 | 56.6 | 9.30E−14 |
| 161 | Mnom000690 | AhpC-TSA | 77 | 211 | 214 | 4.10E−61 |
| 161 | Mnom000690 | 1-cysPrx_C | 221 | 265 | 64.5 | 4.10E−16 |
| 162 | Mnom000107 | PAS_2 | 70 | 186 | 231.5 | 2.10E−66 |
| 162 | Mnom000107 | GAF | 219 | 404 | 105.2 | 2.10E−28 |
| 162 | Mnom000107 | Phytochrome | 415 | 595 | 408.4 | 1.20E−119 |
| 162 | Mnom000107 | PAS | 622 | 737 | 93.7 | 6.60E−25 |
| 162 | Mnom000107 | PAS_4 | 628 | 742 | 18.7 | 0.0033 |
| 162 | Mnom000107 | PAS | 752 | 877 | 101.1 | 3.70E−27 |
| 162 | Mnom000107 | HisKA | 897 | 961 | 27.7 | 4.90E−05 |
| 162 | Mnom000107 | HATPase_c | 1011 | 1123 | 67.2 | 6.00E−17 |
| 163 | Mnom000108 | PAS_2 | 70 | 186 | 231.5 | 2.10E−66 |
| 163 | Mnom000108 | GAF | 219 | 404 | 108.6 | 2.10E−29 |
| 163 | Mnom000108 | Phytochrome | 415 | 595 | 408.4 | 1.20E−119 |
| 163 | Mnom000108 | PAS | 622 | 737 | 93.7 | 6.60E−25 |
| 163 | Mnom000108 | PAS_4 | 628 | 742 | 18.7 | 0.0033 |
| 163 | Mnom000108 | PAS | 752 | 877 | 101.1 | 3.70E−27 |
| 163 | Mnom000108 | HisKA | 897 | 961 | 27.7 | 4.90E−05 |
| 163 | Mnom000108 | HATPase_c | 1011 | 1123 | 67.2 | 6.00E−17 |
| 164 | Mnom000109 | PAS_2 | 70 | 186 | 231.5 | 2.10E−66 |
| 164 | Mnom000109 | GAF | 219 | 404 | 101.3 | 3.20E−27 |
| 164 | Mnom000109 | Phytochrome | 415 | 595 | 408.4 | 1.20E−119 |
| 164 | Mnom000109 | PAS | 622 | 737 | 93.7 | 6.60E−25 |
| 164 | Mnom000109 | PAS_4 | 628 | 742 | 18.7 | 0.0033 |
| 164 | Mnom000109 | PAS | 752 | 877 | 101.1 | 3.70E−27 |
| 164 | Mnom000109 | HisKA | 897 | 961 | 27.7 | 4.90E−05 |
| 164 | Mnom000109 | HATPase_c | 1011 | 1123 | 67.2 | 6.00E−17 |
| 165 | Mnom000110 | PAS_2 | 97 | 217 | 213.1 | 7.30E−61 |
| 165 | Mnom000110 | GAF | 250 | 432 | 111.1 | 3.80E−30 |
| 165 | Mnom000110 | Phytochrome | 443 | 622 | 414.3 | 1.90E−121 |
| 165 | Mnom000110 | PAS | 653 | 769 | 123.6 | 6.20E−34 |
| 165 | Mnom000110 | PAS_4 | 659 | 774 | 28.3 | 3.20E−05 |
| 165 | Mnom000110 | PAS | 784 | 906 | 89.3 | 1.30E−23 |
| 165 | Mnom000110 | HisKA | 926 | 990 | 53.4 | 8.90E−13 |
| 165 | Mnom000110 | HATPase_c | 1038 | 1150 | 50.3 | 7.40E−12 |
| 166 | Mnom000111 | PAS_2 | 97 | 217 | 213.1 | 7.30E−61 |
| 166 | Mnom000111 | GAF | 250 | 432 | 114.7 | 3.00E−31 |
| 166 | Mnom000111 | Phytochrome | 443 | 622 | 414.3 | 1.90E−121 |
| 166 | Mnom000111 | PAS | 1653 | 769 | 123.6 | 6.20E−34 |
| 166 | Mnom000111 | PAS_4 | 659 | 774 | 28.3 | 3.20E−05 |
| 166 | Mnom000111 | PAS | 784 | 906 | 89.3 | 1.30E−23 |
| 166 | Mnom000111 | HisKA | 926 | 990 | 53.4 | 8.90E−13 |
| 166 | Mnom000111 | HATPase_c | 1038 | 1150 | 50.3 | 7.40E−12 |
| 167 | Mnom000112 | PAS_2 | 97 | 217 | 213.1 | 7.30E−61 |
| 167 | Mnom000112 | GAF | 250 | 432 | 110.1 | 7.50E−30 |
| 167 | Mnom000112 | Phytochrome | 443 | 622 | 414.3 | 1.90E−12 |
| 167 | Mnom000112 | PAS | 653 | 769 | 123.6 | 6.20E−34 |
| 167 | Mnom000112 | PAS_4 | 659 | 774 | 28.3 | 3.20E−05 |
| 167 | Mnom000112 | PAS | 784 | 906 | 89.3 | 1.30E−23 |
| 167 | Mnom000112 | HisKA | 926 | 990 | 53.4 | 8.90E−13 |
| 167 | Mnom000112 | HATPase_c | 1038 | 1150 | 50.3 | 7.40E−12 |
| 168 | Mnom000113, | PAS_2 | 70 | 186 | 231.5 | 2.10E−66 |
| | Mnom000116 | | | | | |
| 168 | Mnom000113, | GAF | 219 | 404 | 102.6 | 1.40E−27 |
| | Mnom000116 | | | | | |
| 168 | Mnom000113, | Phytochrome | 415 | 595 | 408.4 | 1.20E−119 |
| | Mnom000116 | | | | | |
| 168 | Mnom000113, | PAS | 622 | 737 | 93.7 | 6.60E−25 |
| | Mnom000116 | | | | | |
| 168 | Mnom000113, | PAS_4 | 628 | 742 | 18.7 | 0.0033 |
| | Mnom000116 | | | | | |
| 168 | Mnom000113, | PAS | 752 | 877 | 101.1 | 3.70E−27 |
| | Mnom000116 | | | | | |
| 168 | Mnom000113, | HisKA | 897 | 961 | 27.7 | 4.90E−05 |
| | Mnom000116 | | | | | |
| 168 | Mnom000113, | HATPase_c | 1011 | 1123 | 67.2 | 6.00E−17 |
| | Mnom000116 | | | | | |
| 169 | Mnom000114, | PAS_2 | 97 | 217 | 213.1 | 7.30E−61 |
| | Mnom000117 | | | | | |
| 169 | Mnom000114, | GAF | 250 | 432 | 112.6 | 1.30E−30 |
| | Mnom000117 | | | | | |
| 169 | Mnom000114, | Phytochrome | 443 | 622 | 414.3 | 1.90E−121 |
| | Mnom000117 | | | | | |
| 169 | Mnom000114, | PAS | 653 | 769 | 123.6 | 6.20E−34 |
| | Mnom000117 | | | | | |
| 169 | Mnom000114, | PAS_4 | 659 | 774 | 28.3 | 3.20E−05 |
| | Mnom000117 | | | | | |
| 169 | Mnom000114, | PAS | 784 | 906 | 89.3 | 1.30E−23 |
| | Mnom000117 | | | | | |
| 169 | Mnom000114, | HisKA | 926 | 990 | 53.4 | 8.90E−13 |
| | Mnom000117 | | | | | |
| 169 | Mnom000114, | HATPase_c | 1038 | 1150 | 50.3 | 7.40E−12 |
| | Mnom000117 | | | | | |
| 170 | Mnom000115, | PAS_2 | 97 | 217 | 213.1 | 7.30E−61 |
| | Mnom000118 | | | | | |
| 170 | Mnom000115, | GAF | 250 | 432 | 115.9 | 1.30E−31 |
| | Mnom000118 | | | | | |
| 170 | Mnom000115, | Phytochrome | 443 | 622 | 414.3 | 1.90E−121 |
| | Mnom000118 | | | | | |
| 170 | Mnom000115, | PAS | 653 | 769 | 123.6 | 6.20E−34 |
| | Mnom000118 | | | | | |
| 170 | Mnom000115, | PAS_4 | 659 | 774 | 28.3 | 3.20E−05 |
| | Mnom000118 | | | | | |
| 170 | Mnom000115, | PAS | 784 | 906 | 89.3 | 1.30E−23 |
| | Mnom000118 | | | | | |
| 170 | Mnom000115, | HisKA | 926 | 990 | 53.4 | 8.90E−13 |
| | Mnom000118 | | | | | |
| 170 | Mnom000115, | HATPase_c | 1038 | 1150 | 50.3 | 7.40E−12 |
| | Mnom000118 | | | | | |
| 171 | Mnom000119 | PAS_2 | 70 | 186 | 231.5 | 2.10E−66 |
| 171 | Mnom000119 | GAF | 219 | 404 | 107 | 6.20E−29 |
| 171 | Mnom000119 | Phytochrome | 415 | 595 | 408.4 | 1.20E−119 |
| 171 | Mnom000119 | PAS | 622 | 737 | 93.7 | 6.60E−25 |
| 171 | Mnom000119 | PAS_4 | 628 | 742 | 18.7 | 0.0033 |
| 171 | Mnom000119 | PAS | 752 | 877 | 101.1 | 3.70E−27 |
| 171 | Mnom000119 | HisKA | 897 | 961 | 27.7 | 4.90E−05 |
| 171 | Mnom000119 | HATPase_c | 1011 | 1123 | 67.2 | 6.00E−17 |
| 172 | Mnom000738 | DUF260 | 7 | 108 | 203.5 | 5.70E−58 |
| 175 | Mnom000857, | DUF231 | 364 | 542 | 272.2 | 1.20E−78 |
| | Mnom000858, | | | | | |
| | Mnom000860 | | | | | |
| 176 | Mnom000873 | Glyco_hydro_18 | 27 | 306 | 45.5 | 1.70E−10 |
| 177 | Mnom000886 | Synaptobrevin | 132 | 238 | 56.1 | 1.40E−13 |
| 178 | Mnom000923-Mnom000924 | PAS_2 | 97 | 217 | 213.1 | 7.30E−61 |
| 178 | Mnom000923-Mnom000924 | GAF | 250 | 432 | 115.9 | 1.30E−31 |
| 178 | Mnom000923-Mnom000924 | Phytochrome | 443 | 622 | 406.8 | 3.60E−119 |
| 178 | Mnom000923-Mnom000924 | PAS | 653 | 769 | 123.6 | 6.20E−34 |
| 178 | Mnom000923-Mnom000924 | PAS_4 | 659 | 774 | 28.3 | 3.20E−05 |
| 178 | Mnom000923-Mnom000924 | PAS | 784 | 906 | 89.3 | 1.30E−23 |
| 178 | Mnom000923-Mnom000924 | HisKA | 926 | 990 | 53.4 | 8.90E−13 |
| 178 | Mnom000923-Mnom000924 | HATPase_c | 1038 | 1150 | 50.3 | 7.40E−12 |
| 179 | Mnom000937 | PAS | 116 | 230 | 22.8 | 0.0014 |
| 179 | Mnom000937 | PAS_3 | 141 | 227 | 19.9 | 0.0011 |
| 179 | Mnom000937 | PAS | 390 | 504 | 10.5 | 0.047 |
| 179 | Mnom000937 | Pkinase | 582 | 870 | 291.4 | 2.00E−84 |
| 179 | Mnom000937 | Pkinase_Tyr | 582 | 877 | −58.7 | 8.00E−10 |
| 180 | Mnom000942, | TP6A_N | 89 | 156 | 84.6 | 3.50E−22 |
| | Mnom000955-Mnom000956 | | | | | |
| 181 | Mnom000985 | Response_reg | 16 | 128 | 26.4 | 5.90E−06 |
| 183 | Mnom001055 | Peptidase_S10 | 42 | 455 | 562.2 | 6.00E−166 |
| 184 | Mnom001090-Mnom001092 | Gln-synt_N | 31 | 111 | 149.1 | 1.30E−41 |
| 184 | Mnom001090-Mnom001092 | Gln-synt_C | 117 | 369 | 295.8 | 9.50E−86 |
| 185 | Mnom001138 | UDPGP | 26 | 443 | 913.5 | 1.00E−271 |
| 186 | Mnom001140 | DnaJ | 11 | 80 | 75 | 2.80E−19 |
| 187 | Mnom001141 | DnaJ | 12 | 78 | 109 | 1.60E−29 |
| 188 | Mnom001142 | NAPRTase | 175 | 439 | 150.5 | 5.10E−42 |
| 189 | Mnom001146 | DUF296 | 114 | 234 | 180.6 | 4.40E−51 |
| 190 | Mnom001147 | DUF296 | 114 | 234 | 180.6 | 4.40E−51 |
| 191 | Mnom001160 | zf-A20 | 14 | 38 | 34.2 | 5.10E−07 |
| 191 | Mnom001160 | zf-AN1 | 115 | 155 | 72.8 | 1.20E−18 |
| 192 | Mnom001162 | Cellulose_synt | 284 | 1107 | 1889.4 | 0 |
| 193 | PHE0000597 | Mannitol_dh | 10 | 132 | 181.9 | 1.80E−51 |
| 193 | PHE0000597 | Mannitol_dh_C | 157 | 383 | 394.4 | 1.90E−115 |
| 194 | PHE0001556 | RimK | 226 | 486 | −20.7 | 1.20E−05 |
| 194 | PHE0001556 | Mur_ligase_M | 498 | 711 | 138.1 | 2.70E−38 |
| 194 | PHE0001556 | Mur_ligase_C | 735 | 821 | 75.8 | 1.60E−19 |
| 195 | PHE0001557 | RimK | 217 | 370 | −48.2 | 0.0014 |
| 195 | PHE0001557 | Mur_ligase_M | 491 | 704 | 138.5 | 2.10E−38 |
| 195 | PHE0001557 | Mur_ligase_C | 728 | 813 | 80.8 | 5.00E−21 |
| 196 | PHE0001559 | Peptidase_S51 | 52 | 212 | 44.4 | 4.40E−10 |
| 198 | PHE0003348 | HLH | 310 | 359 | 70 | 8.90E−18 |
| 199 | PHE0003706 | SSF | 54 | 471 | −94.5 | 2.00E−05 |
| 200 | PHE0003708 | SSF | 46 | 463 | −112.6 | 8.80E−05 |
| 201 | PHE0004230 | E2F_TDP | 227 | 292 | 134 | 4.90E−37 |
| 202 | PHE0004820 | HLH | 113 | 162 | 62.1 | 2.10E−15 |
| 204 | PHE0006017 | Na_H_Exchanger | 26 | 445 | 173.4 | 6.40E−49 |
| 205 | PHE0006102 | Brix | 27 | 342 | 311.1 | 2.30E−90 |
| 207 | PHE0006380 | RNase_PH_C | 129 | 201 | 61.9 | 2.40E−15 |
| 209 | PHE0006610 | GSHPx | 119 | 227 | 234.2 | 3.30E−67 |
| 210 | PHE0006613 | GSHPx | 29 | 137 | 200.6 | 4.10E−57 |
| 211 | PHE0006916 | RAMP4 | 49 | 112 | 130 | 7.80E−36 |
| 213 | PHE0007643 | LRRNT_2 | 38 | 79 | 39.3 | 1.60E−08 |
| 213 | PHE0007643 | LRR_1 | 155 | 177 | 12.5 | 1.2 |
| 213 | PHE0007643 | LRR_1 | 179 | 201 | 15 | 0.31 |
| 213 | PHE0007643 | LRR_1 | 227 | 249 | 16.4 | 0.12 |
| 213 | PHE0007643 | Pkinase_Tyr | 429 | 696 | 0.6 | 9.10E−14 |
| 216 | PHE0008411 | DUF778 | 41 | 162 | 269.1 | 1.00E−77 |
| 218 | PHE0008602 | Gln-synt_N | 103 | 184 | 168.6 | 1.80E−47 |
| 218 | PHE0008602 | Gln-synt_C | 191 | 472 | 579.1 | 5.00E−171 |
| 219 | PHE0009339 | HSP70 | 66 | 673 | 1343.3 | 0 |
| 220 | PHE0009341 | TrkH | 211 | 569 | 125.4 | 1.80E−34 |
| 221 | PHE0010195 | PTR2 | 85 | 432 | 70 | 8.70E−18 |
| 222 | PHE0010352 | CBFD_NFYB_HMF | 31 | 96 | 132.4 | 1.40E−36 |
| 223 | PHE0010354 | CBFD_NFYB_HMF | 34 | 99 | 128.8 | 1.80E−35 |
| 224 | PHE0010646 | Spermine_synth | 46 | 291 | 500.6 | 2.10E−147 |
| 225 | PHE0010648 | SNF2_N | 25 | 373 | −19.4 | 1.30E−07 |
| 225 | PHE0010648 | Helicase_C | 441 | 520 | 59.4 | 1.40E−14 |
| 226 | PHE0010649 | ResIII | 18 | 230 | 2.1 | 3.60E−05 |
| 226 | PHE0010649 | SNF2_N | 24 | 374 | 4.4 | 5.60E−09 |
| 226 | PHE0010649 | Helicase_C | 442 | 521 | 56.7 | 8.90E−14 |
| 227 | PHE0010852 | HLH | 403 | 451 | 40.3 | 7.80E−09 |
| 228 | PHE0010956 | Nucleoside_tran | 167 | 450 | 514.2 | 1.70E−151 |
| 230 | PHE0011063 | Globin | 49 | 145 | 53 | 1.20E−12 |
| 231 | PHE0011543 | zf-B_box | 4 | 50 | 27.5 | 5.50E−05 |
| 232 | PHE0013202 | HLH | 172 | 221 | 22.2 | 0.00075 |
| 233 | PHE0013203 | SAM_2 | 236 | 302 | 54.6 | 3.70E−13 |
| 233 | PHE0013203 | SAM_1 | 237 | 300 | 85.7 | 1.60E−22 |
| 233 | PHE0013203 | DRMBL | 609 | 707 | 160.2 | 6.20E−45 |
| 235 | PHE0013292 | PDZ | 220 | 304 | 34.6 | 4.00E−07 |
| 235 | PHE0013292 | Peptidase_S41 | 340 | 503 | 244.8 | 2.20E−70 |
| 238 | PHE0013734 | SRF-TF | 9 | 59 | 64.9 | 3.00E−16 |
| 239 | TRG0000049 | CBFD_NFYB_HMF | 75 | 140 | 100.2 | 7.10E−27 |
| 241 | TRG0000332 | CBFB_NFYA | 192 | 248 | 141.2 | 3.30E−39 |
| 242 | 1071693 | PAS_2 | 98 | 218 | 192.3 | 1.30E−54 |
| 242 | 1071693 | GAF | 251 | 433 | 116.9 | 6.70E−32 |
| 242 | 1071693 | Phytochrome | 444 | 623 | 409.1 | 7.40E−120 |
| 242 | 1071693 | PAS | 654 | 738 | 30.7 | 5.80E−06 |

TABLE 10
Pfam module annotation

| PEP | | | |
|---|---|---|---|
| SEQ | | | |
| ID NO | Gene ID | Pfam domain module | Position |
| 123 | Mnom000202 | Copine | 116-264 |
| 124 | Mnom000215-Mnom000216 | CBFB_NFYA | 97-153 |
| 125 | Mnom000219-Mnom000220 | CBFD_NFYB_HMF | 25-90 |
| 126 | Mnom000256-Mnom000257 | Glu_synthase | 115-494 |
| 127 | Mnom000260-Mnom000261 | HEAT::HEAT::FAT::Rapamycin_bind:: | 183-219::716-753::1435-1801:: |
| | | PI3_PI4_kinase::FAT C | 1908-2007::2074-2324:: |
| | | | 2432-2464 |
| 128 | Mnom000262 | F-box | 29-76 |
| 129 | Mnom000324 | AP2 | 136-188 |
| 130 | Mnom000372 | SAP::zf-MIZ | 34-68::357-406 |
| 131 | Mnom000412-Mnom000413 | 14-3-3 | 4-219 |
| 132 | Mnom000444 | GATase_2::Glu_syn_central::Glu_synthase:: | 39-409::483-784::841-1226:: |
| | | GXGXG | 1304-1499 |
| 134 | Mnom000471 | Glyco_transf_20::Trehalose_PPase | 16-482::572-819 |
| 135 | Mnom000480 | Response_reg | 16-128 |
| 137 | Mnom000485 | FAR1::FAR1 | 67-160::204-298 |
| 139 | Mnom000490 | PTR2 | 1-257 |
| 140 | Mnom000491 | PPR::PPR::PPR::PPR::PPR::PPR:: | 151-185::186-219::220-254:: |
| | | PPR::PPR::PPR::PPR::PPR | 258-292::293-327::328-362:: |
| | | | 363-397::398-432::434-468:: |
| | | | 469-503::534-568 |
| 142 | Mnom000496 | Efhand | 109-137 |
| 145 | Mnom000517 | 14-3-3 | 8-225 |
| 146 | Mnom000554 | DUF761 | 9-329 |
| 147 | Mnom000570 | SAP::PHD::zf-MIZ | 11-45::114-168::359-408 |
| 148 | Mnom000580 | HSP20 | 133-230 |
| 149 | Mnom000616-Mnom000617 | Fer2::Fer2_2::FAD_binding_5:: | 19-88::98-173::260-440::447-558:: |
| | | CO_deh_flav_C::Ald_Xan_dh_C:: | 612-719::727-1272 |
| | | Ald_Xan_dh_C2 | |
| 150 | Mnom000618-Mnom000619 | Fer2::Fer2_2::FAD_binding_5:: | 11-80::90-165::252-432::439-550:: |
| | | CO_deh_flav_C::Ald_Xan_dh_C:: | 604-711::719-1264 |
| | | Ald_Xan_dh_C2 | |
| 151 | Mnom000626-Mnom000627 | 2OG-FeII_Oxy | 180-280 |
| 152 | Mnom000628 | 2OG-FeII_Oxy | 180-280 |
| 154 | Mnom000637 | B3::Auxin_resp::AUX_IAA | 312-417::439-524::681-826 |
| 156 | Mnom000641 | XRN_N::zf-CCHC | 1-256::263-280 |
| 157 | Mnom000643 | PALP | 36-329 |
| 158 | Mnom000645 | GMC_oxred_N | 210-434 |
| 159 | Mnom000647 | Peptidase_S10 | 42-455 |
| 160 | Mnom000682 | Glycos_transf_1 | 296-459 |
| 161 | Mnom000690 | AhpC-TSA::1-cysPrx_C | 77-211::221-265 |
| 162 | Mnom000107 | PAS_2::GAF::Phytochrome::PAS:: | 70-186::219-404::415-595:: |
| | | PAS::HisKA::HATPase_c | 622-737::752-877::897-961::1011-1123 |
| 163 | Mnom000108 | PAS_2::GAF::Phytochrome::PAS:: | 70-186::219-404::415-595:: |
| | | PAS::HisKA::HATPase_c | 622-737::752-877::897-961::1011-1123 |
| 164 | Mnom000109 | PAS_2::GAF::Phytochrome::PAS:: | 70-186::219-404::415-595:: |
| | | PAS::HisKA::HATPase_c | 622-737::752-877::897-961::1011-1123 |
| 165 | Mnom000110 | PAS_2::GAF::Phytochrome::PAS:: | 97-217::250-432::443-622:: |
| | | PAS::HisKA::HATPase_c | 653-769::784-906::926-990::1038-1150 |
| 166 | Mnom000111 | PAS_2::GAF::Phytochrome::PAS:: | 97-217::250-432::443-622:: |
| | | PAS::HisKA::HATPase_c | 653-769::784-906::926-990::1038-1150 |
| 167 | Mnom000112 | PAS_2::GAF::Phytochrome::PAS:: | 97-217::250-432::443-622:: |
| | | PAS::HisKA::HATPase_c | 653-769::784-906::926-990::1038-1150 |
| 168 | Mnom000113, | PAS_2::GAF::Phytochrome::PAS:: | 70-186::219-404::415-595:: |
| | Mnom000116 | PAS::HisKA::HATPase_c | 622-737::752-877::897-961::1011-1123 |
| 169 | Mnom000114, | PAS_2::GAF::Phytochrome::PAS:: | 97-217::250-432::443-622:: |
| | Mnom000117 | PAS::HisKA::HATPase_c | 653-769::784-906::926-990::1038-1150 |
| 170 | Mnom000115, | PAS_2::GAF::Phytochrome::PAS:: | 97-217::250-432::443-622:: |
| | Mnom000118 | PAS::HisKA::HATPase_c | 653-769::784-906::926-990::1038-1150 |
| 171 | Mnom000119 | PAS_2::GAF::Phytochrome::PAS:: | 70-186::219-404::415-595:: |
| | | PAS::HisKA::HATPase_c | 622-737::752-877::897-961::1011-1123 |
| 172 | Mnom000738 | DUF260 | 7-108 |
| 175 | Mnom000857, | DUF231 | 364-542 |
| | Mnom000858, | | |
| | Mnom000860 | | |
| 176 | Mnom000873 | Glyco_hydro_18 | 27-306 |
| 177 | Mnom000886 | Synaptobrevin | 132-238 |
| 178 | Mnom000923-Mnom000924 | PAS_2::GAF::Phytochrome::PAS:: | 97-217::250-432::443-622:: |
| | | PAS::HisKA::HATPase_c | 653-769::784-906::926-990::1038-1150 |
| 179 | Mnom000937 | PAS_3::PAS::Pkinase | 141-227::390-504::582-870 |
| 180 | Mnom000942, | TP6A_N | 89-156 |
| | Mnom000955-Mnom000956 | | |
| 181 | Mnom000985 | Response_reg | 16-128 |
| 183 | Mnom001055 | Peptidase_S10 | 42-455 |
| 184 | Mnom001090-Mnom001092 | Gln-synt_N::Gln-synt_C | 31-111::117-369 |
| 185 | Mnom001138 | UDPGP | 26-443 |
| 186 | Mnom001140 | DnaJ | 11-80 |
| 187 | Mnom001141 | DnaJ | 12-78 |
| 188 | Mnom001142 | NAPRTase | 175-439 |
| 189 | Mnom001146 | DUF296 | 114-234 |
| 190 | Mnom001147 | DUF296 | 114-234 |
| 191 | Mnom001160 | zf-A20::zf-AN1 | 14-38::115-155 |
| 192 | Mnom001162 | Cellulose_synt | 284-1107 |
| 193 | PHE0000597 | Mannitol_dh::Mannitol_dh_C | 10-132::157-383 |
| 194 | PHE0001556 | RimK::Mur_ligase_M::Mur_ligase_C | 226-486::498-711::735-821 |
| 195 | PHE0001557 | RimK::Mur_ligase_M::Mur_ligase_C | 217-370::491-704::728-813 |
| 196 | PHE0001559 | Peptidase_S51 | 52-212 |
| 198 | PHE0003348 | HLH | 310-359 |
| 199 | PHE0003706 | SSF | 54-471 |
| 200 | PHE0003708 | SSF | 46-463 |
| 201 | PHE0004230 | E2F_TDP | 227-292 |
| 202 | PHE0004820 | HLH | 113-162 |
| 204 | PHE0006017 | Na_H_Exchanger | 26-445 |
| 205 | PHE0006102 | Brix | 27-342 |
| 207 | PHE0006380 | RNase_PH_C | 129-201 |
| 209 | PHE0006610 | GSHPx | 119-227 |
| 210 | PHE0006613 | GSHPx | 29-137 |
| 211 | PHE0006916 | RAMP4 | 49-112 |
| 213 | PHE0007643 | LRRNT_2::LRR_1::LRR_1::LRR_1:: | 38-79::155-177::179-201:: |
| | | Pkinase_Tyr | 227-249::429-696 |
| 216 | PHE0008411 | DUF778 | 41-162 |
| 218 | PHE0008602 | Gln-synt_N::Gln-synt_C | 103-184::191-472 |
| 219 | PHE0009339 | HSP70 | 66-673 |
| 220 | PHE0009341 | TrkH | 211-569 |
| 221 | PHE0010195 | PTR2 | 85-432 |
| 222 | PHE0010352 | CBFD_NFYB_HMF | 31-96 |
| 223 | PHE0010354 | CBFD_NFYB_HMF | 34-99 |
| 224 | PHE0010646 | Spermine_synth | 46-291 |
| 225 | PHE0010648 | SNF2_N::Helicase_C | 25-373::441-520 |
| 226 | PHE0010649 | SNF2_N::Helicase_C | 24-374::442-521 |
| 227 | PHE0010852 | HLH | 403-451 |
| 228 | PHE0010956 | Nucleoside_tran | 167-450 |
| 230 | PHE0011063 | Globin | 49-145 |
| 231 | PHE0011543 | zf-B_box | 4-50 |
| 232 | PHE0013202 | HLH | 172-221 |
| 233 | PHE0013203 | SAM_1::DRMBL | 237-300::609-707 |
| 235 | PHE0013292 | PDZ::Peptidase_S41 | 220-304::340-503 |
| 238 | PHE0013734 | SRF-TF | 9-59 |
| 239 | TRG0000049 | CBFD_NFYB_HMF | 75-140 |
| 241 | TRG0000332 | CBFB_NFYA | 192-248 |
| 242 | 1071693 | PAS_2::GAF::Phytochrome::PAS | 98-218::251-433::444-623::654-738 |

TABLE 11
Description of Pfam domains

| | Accession | Gathering | |
|---|---|---|---|
| Pfam domain name | number | cutoff | Domain description |
| 14-3-3 | PF00244.12 | 25.0000; | 14-3-3 protein |
| 1-cysPrx_C | PF10417.1 | 36.0000; | C-terminal domain of 1-Cys peroxiredoxin |
| 2OG-FeII_Oxy | PF03171.12 | 11.5000; | 2OG-Fe(II) oxygenase superfamily |
| AhpC-TSA | PF00578.13 | 4.8000; | AhpC/TSA family |
| Ald_Xan_dh_C | PF01315.14 | −7.6000; | Aldehyde oxidase and xanthine |
| | | | dehydrogenase, a/b hammerhead domain |
| Ald_Xan_dh_C2 | PF02738.10 | −233.0000; | Aldehyde oxidase and xanthine |
| | | | dehydrogenase, molybdopterin binding |
| | | | domain |
| AP2 | PF00847.12 | 21.7000; | AP2 domain |
| AUX_IAA | PF02309.8 | −83.0000; | AUX/IAA family |
| Auxin_resp | PF06507.5 | 25.0000; | Auxin response factor |
| B3 | PF02362.13 | 26.5000; | B3 DNA binding domain |
| Brix | PF04427.10 | 0.1000; | Brix domain |
| CBFB_NFYA | PF02045.7 | 25.0000; | CCAAT-binding transcription factor (CBF- |
| | | | B/NF-YA) subunit B |
| CBFD_NFYB_HMF | PF00808.15 | 18.4000; | Histone-like transcription factor (CBF/NF-Y) |
| | | | and archaeal histone |
| Cellulose_synt | PF03552.6 | −257.9000; | Cellulose synthase |
| CO_deh_flav_C | PF03450.9 | −1.0000; | CO dehydrogenase flavoprotein C-terminal |
| | | | domain |
| Copine | PF07002.8 | −36.5000; | Copine |
| DAO | PF01266.16 | −34.9000; | FAD dependent oxidoreductase |
| DnaJ | PF00226.23 | −8.0000; | DnaJ domain |
| DRMBL | PF07522.6 | 25.0000; | DNA repair metallo-beta-lactamase |
| DUF231 | PF03005.7 | −46.3000; | Arabidopsis proteins of unknown function |
| DUF260 | PF03195.6 | −30.4000; | Protein of unknown function DUF260 |
| DUF296 | PF03479.7 | −3.0000; | Domain of unknown function (DUF296) |
| DUF761 | PF05553.3 | −97.7000; | Cotton fibre expressed protein |
| DUF778 | PF05608.4 | −38.6000; | Protein of unknown function (DUF778) |
| E2F_TDP | PF02319.12 | 17.0000; | E2F/DP family winged-helix DNA-binding |
| | | | domain |
| Efhand | PF00036.24 | 21.7000; | EF hand |
| FAD_binding_5 | PF00941.13 | −57.4000; | FAD binding domain in molybdopterin |
| | | | dehydrogenase |
| FAR1 | PF03101.7 | −12.7000; | FAR1 DNA-binding domain |
| FAT | PF02259.15 | −64.1000; | FAT domain |
| FATC | PF02260.12 | 16.5000; | FATC domain |
| F-box | PF00646.25 | 14.7000; | F-box domain |
| Fer2 | PF00111.19 | 11.2000; | 2Fe—2S iron-sulfur cluster binding domain |
| Fer2_2 | PF01799.12 | −19.5000; | [2Fe—2S] binding domain |
| GAF | PF01590.18 | 23.0000; | GAF domain |
| GATase_2 | PF00310.13 | −91.6000; | Glutamine amidotransferases class-II |
| Gln-synt_C | PF00120.16 | −124.0000; | Glutamine synthetase, catalytic domain |
| Gln-synt_N | PF03951.11 | 9.0000; | Glutamine synthetase, beta-Grasp domain |
| Globin | PF00042.14 | −7.3000; | Globin |
| Glu_syn_central | PF04898.6 | 25.0000; | Glutamate synthase central domain |
| Glu_synthase | PF01645.9 | −198.4000; | Conserved region in glutamate synthase |
| Glyco_hydro_18 | PF00704.20 | 13.0000; | Glycosyl hydrolases family 18 |
| Glyco_transf_20 | PF00982.13 | −243.6000; | Glycosyltransferase family 20 |
| Glycos_transf_1 | PF00534.12 | −7.3000; | Glycosyl transferases group 1 |
| GMC_oxred_N | PF00732.11 | −71.1000; | GMC oxidoreductase |
| GSHPx | PF00255.11 | −16.0000; | Glutathione peroxidase |
| GXGXG | PF01493.11 | 5.0000; | GXGXG motif |
| HATPase_c | PF02518.18 | 22.4000; | Histidine kinase-, DNA gyrase B-, and |
| | | | HSP90-like ATPase |
| HEAT | PF02985.14 | 16.4000; | HEAT repeat |
| Helicase_C | PF00271.23 | 6.9000; | Helicase conserved C-terminal domain |
| HisKA | PF00512.17 | 22.1000; | His Kinase A (phosphoacceptor) domain |
| HLH | PF00010.18 | 8.3000; | Helix-loop-helix DNA-binding domain |
| HSP20 | PF00011.13 | 13.0000; | Hsp20/alpha crystallin family |
| HSP70 | PF00012.12 | −286.0000; | Hsp70 protein |
| LRR_1 | PF00560.25 | 10.9000; | Leucine Rich Repeat |
| LRRNT_2 | PF08263.4 | 18.6000; | Leucine rich repeat N-terminal domain |
| Mannitol_dh | PF01232.15 | −10.5000; | Mannitol dehydrogenase Rossmann domain |
| Mannitol_dh_C | PF08125.5 | −79.1000; | Mannitol dehydrogenase C-terminal domain |
| Mur_ligase_C | PF02875.13 | 8.0000; | Mur ligase family, glutamate ligase domain |
| Mur_ligase_M | PF08245.4 | −44.7000; | Mur ligase middle domain |
| Na_H_Exchanger | PF00999.13 | −67.9000; | Sodium/hydrogen exchanger family |
| NAPRTase | PF04095.8 | −88.5000; | Nicotinate phosphoribosyltransferase |
| | | | (NAPRTase) family |
| Nucleoside_tran | PF01733.10 | −85.0000; | Nucleoside transporter |
| PALP | PF00291.17 | −70.0000; | Pyridoxal-phosphate dependent enzyme |
| PAS | PF00989.16 | 0.0000; | PAS fold |
| PAS_2 | PF08446.3 | −2.1000; | PAS fold |
| PAS_3 | PF08447.3 | 17.2000; | PAS fold |
| PAS_4 | PF08448.2 | 16.4000; | PAS fold |
| PDZ | PF00595.16 | 20.7000: | PDZ domain (Also known as DHR or GLGF) |
| Peptidase_S10 | PF00450.14 | −198.0000; | Serine carboxypeptidase |
| Peptidase_S41 | PF03572.10 | −25.8000; | Peptidase family S41 |
| Peptidase_S51 | PF03575.9 | −11.1000; | Peptidase family S51 |
| PHD | PF00628.21 | 26.2000; | PHD-finger |
| Phytochrome | PF00360.12 | 13.0000; | Phytochrome region |
| PI3_PI4_kinase | PF00454.19 | 14.8000; | Phosphatidylinositol 3- and 4-kinase |
| Pkinase | PF00069.17 | 70.3000; | Protein kinase domain |
| Pkinase_Tyr | PF07714.9 | −155.0000; | Protein tyrosine kinase |
| PPR | PF01535.12 | 1.6000; | PPR repeat |
| PTR2 | PF00854.13 | −50.0000; | POT family |
| RAMP4 | PF06624.4 | 25.0000; | Ribosome associated membrane protein |
| | | | RAMP4 |
| Rapamycin_bind | PF08771.3 | 25.0000; | Rapamycin binding domain |
| Redoxin | PF08534.2 | 8.9000; | Redoxin |
| ResIII | PF04851.7 | 0.8000; | Type III restriction enzyme, res subunit |
| Response_reg | PF00072.16 | 4.1000; | Response regulator receiver domain |
| RimK | PF08443.3 | −56.1000; | RimK-like ATP-grasp domain |
| RNase_PH_C | PF03725.7 | 20.0000; | 3′ exoribonuclease family, domain 2 |
| SAM_1 | PF00536.22 | 11.3000; | SAM domain (Sterile alpha motif) |
| SAM_2 | PF07647.9 | 0.0000; | SAM domain (Sterile alpha motif) |
| SAP | PF02037.19 | 1.5000; | SAP domain |
| SNF2_N | PF00176.15 | −72.0000; | SNF2 family N-terminal domain |
| Spermine_synth | PF01564.9 | −93.8000; | Spermine/spermidine synthase |
| SRF-TF | PF00319.10 | 11.0000; | SRF-type transcription factor (DNA-binding |
| | | | and dimerisation domain) |
| SSF | PF00474.9 | −162.3000; | Sodium:solute symporter family |
| Synaptobrevin | PF00957.13 | 25.0000; | Synaptobrevin |
| TP6A_N | PF04406.6 | 25.0000; | Type IIB DNA topoisomerase |
| Trehalose_PPase | PF02358.8 | −49.4000; | Trehalose-phosphatase |
| TrkH | PF02386.8 | −96.5000; | Cation transport protein |
| UDPGP | PF01704.10 | −265.2000; | UTP--glucose-1-phosphate |
| | | | uridylyltransferase |
| XRN_N | PF03159.10 | 25.0000; | XRN 5′-3′ exonuclease N-terminus |
| zf-A20 | PF01754.8 | 25.0000; | A20-like zinc finger |
| zf-AN1 | PF01428.8 | 0.0000; | AN1-like Zinc finger |
| zf-B_box | PF00643.16 | 15.3000; | B-box zinc finger |
| zf-CCHC | PF00098.15 | 17.9000: | Zinc knuckle |
| zf-MIZ | PF02891.12 | −2.2000; | MIZ/SP-RING zinc finger |

1. A recombinant DNA construct comprising a polynucleotide encoding a protein that has an amino acid sequence having at least 95% identity over at least 95% of the length of a reference sequence selected from the group consisting of SEQ ID NO: 122-242 when said amino acid sequence is aligned with said reference sequence.

2. A recombinant DNA construct comprising a promoter that is functional in a plant cell and that is operably linked to a polynucleotide that, when expressed in a plant cell:
(a) encodes a protein:
i) having an amino acid sequence selected from the group consisting of SEQ ID NO: 123-140, 145-151, 153-173, 175-187, 189, 191-202, 204-207, 209-238, and 242;
ii) having an amino acid sequence having at least 95% identity over at least 95% of a reference sequence selected from the group consisting of SEQ ID NO: 123-140, 145-151, 153-173, 175-187, 189, 191-202, 204-207, 209-238, and 242 when said amino acid sequence is aligned to said reference sequence; or
iii) that is a homolog of a protein with an amino acid sequence selected from the group consisting of SEQ ID NO: 123-140, 145-151, 153-173, 175-187, 189, 191-202, 204-207, 209-238, and 242; or
(b) is transcribed into an RNA molecule that suppresses the level of an endogenous protein in said plant cell wherein said endogenous protein has an amino acid sequence selected from the group consisting of SEQ ID NO: 122, 141-144, 152, 174, 188, 190, 203, 208, or 239-241 or is a homolog thereof;
wherein said construct is stably integrated into plant chromosomal DNA.

3. A transgenic plant cell comprising the recombinant DNA construct of claim 2 wherein said plant cell is in a plant selected by screening a population of transgenic plants that have been transformed with said construct for an enhanced trait as compared to control plants; and wherein said enhanced trait is enhanced water use efficiency, enhanced cold tolerance, increased yield, enhanced nitrogen use efficiency, enhanced seed protein or enhanced seed oil.

4. A mixture comprising plant cells of claim 3 and an antibody to a protein produced in said cells wherein said protein has an amino acid sequence that has at least 95% identity over at least 95% of the length of a reference sequence selected from the group consisting of SEQ ID NO: 122-242 when said amino acid sequence is aligned to said reference sequence.

5. The plant cell of claim 3 further comprising DNA expressing a protein that provides tolerance from exposure to an herbicide comprising an agent applied at levels that are lethal to a wild type of said plant cell.

6. The plant cell of claim 5 wherein the agent of said herbicide is a glyphosate, dicamba, or glufosinate compound.

7. A transgenic plant comprising a plurality of plant cells of claim 3.

8. The transgenic plant of claim 7 which is homozygous for said recombinant DNA.

9. A transgenic seed comprising a plurality of plant cells of claim 3.

10. The transgenic seed of claim 9 from a corn, soybean, cotton, canola, alfalfa, wheat, rice, sugarcane, or sugar beet plant.

11. Grain comprising transgenic seed identifiable by the recombinant DNA construct of claim 2.

12. Seed meal produced from transgenic seed identifiable by the recombinant DNA construct of claim 2.

13. A transgenic pollen grain comprising a haploid derivative of a plant cell nucleus having a chromosome comprising the recombinant DNA construct of claim 2.

14. A method for manufacturing non-natural, transgenic seed that can be used to produce a crop of transgenic plants with an enhanced trait resulting from expression of the stably-integrated, recombinant DNA construct of claim 2, said method comprising:
(a) screening a population of plants for said enhanced trait and said recombinant DNA, wherein individual plants in said population exhibit said trait at a level less than, essentially the same as or greater than the level that said trait is exhibited in control plants which do not contain said recombinant DNA, wherein said enhanced trait is selected from the group of enhanced traits consisting of enhanced water use efficiency, enhanced cold tolerance, increased yield, enhanced nitrogen use efficiency, enhanced seed protein and enhanced seed oil;
(b) selecting from said population one or more plants that exhibit said trait at a level greater than the level that said trait is exhibited in control plants, and
(c) collecting seed from selected plant from step b.

15. The method of claim 14 wherein said method for manufacturing said transgenic seed further comprises:
(a) verifying that said recombinant DNA is stably integrated in said selected plants, and
(b) analyzing tissue of said selected plant to determine the expression or suppression of a protein having the function of a protein having an amino acid sequence selected from the group consisting of one of SEQ ID NOs: 122-242.

16. The method of claim 15 wherein said seed is corn, soybean, cotton, canola, alfalfa, wheat, rice, sugarcane, or sugar beet seed.

17. A method of producing hybrid corn seed comprising:
(a) acquiring hybrid corn seed from an herbicide tolerant corn plant which also has the stably-integrated, recombinant DNA construct of claim 2;
(b) producing corn plants from said hybrid corn seed, wherein a fraction of the plants produced from said hybrid corn seed is homozygous for said recombinant DNA, a fraction of the plants produced from said hybrid corn seed is hemizygous for said recombinant DNA, and a fraction of the plants produced from said hybrid corn seed has none of said recombinant DNA;
(c) selecting corn plants which are homozygous and hemizygous for said recombinant DNA by treating with an herbicide;
(d) collecting seed from herbicide-treated-surviving corn plants and planting said seed to produce further progeny corn plants;
(e) repeating steps (c) and (d) at least once to produce an inbred corn line; and
(f) crossing said inbred corn line with a second corn line to produce hybrid seed.
